(19) United States
(12) Patent Application Publication
(10) Pub. No.: US 2025/0074970 A1
(43) Pub. Date: Mar. 6, 2025

(54) METHOD OF PURIFYING IMMUNOGLOBULIN G AND USES THEREOF

(71) Applicant: CSL Behring AG, Bern (CH)

(72) Inventors: Roopsee ANAND, Coelbe (DE); Peter DOLLINGER, Weimar (DE); Laura HOLLER, Boca Raton, FL (US); Martin NEUENFELDT, Marburg (DE); Magdalena POLATYNSKA, Marburg (DE); Heike Nicole WILKA, Lahntal (DE); Katrin ANDERS, Marburg (DE); Norbert SCHULZE, Hattersheim (DE); David BOEREMA, Bourbonnais, IL (US)

(21) Appl. No.: 18/293,120

(22) PCT Filed: Jul. 29, 2022
(86) PCT No.: PCT/IB2022/057039

§ 371 Date: Jan. 29, 2024

Related U.S. Application Data
(60) Provisional application No. 63/227,329, filed on Jul. 29, 2021.
(60) Provisional application No. 63/365,530, filed on May 31, 2022.

(30) Foreign Application Priority Data
Jul. 29, 2021 (AU) 2021902332

(51) Int. Cl. C07K 16/06; C07K 1/22

(52) U.S. Cl. CPC C07K 16/065; C07K 1/22

(57) ABSTRACT

The present disclosure relates to methods of purifying immunoglobulin G (IgG) and other proteins, such as albumin, from plasma or a fraction thereof using an affinity chromatography resin comprising a ligand capable of specifically binding to a CH3 domain of human IgG. The present disclosure also relates to formulation and uses of plasma protein product produced from the method.

### RELATED APPLICATION DATA

This application claims priority from Australian Patent Application No 2021902332 filed on 29 Jul. 2021 and entitled “Method of purifying immunoglobulin G and uses thereof”, U.S. Patent Application No. 63/227,329 filed on 29 Jul. 2021 and entitled “Method of purifying immunoglobulin G and uses thereof” and U.S. Patent Application No. 63/365,530 filed on 31 May 2022 and entitled “Method of purifying immunoglobulin G and uses thereof”.

### SEQUENCE LISTING

The present application is filed together with a Sequence Listing in electronic form. The entire contents of the Sequence Listing are hereby incorporated by reference.

### FIELD

The present disclosure relates to methods of purifying immunoglobulin G (IgG) and other proteins, such as albumin, from plasma, and formulations and uses of the plasma protein product thereof.

### BACKGROUND

Immunoglobulin G (IgG) is one of the most abundant proteins in plasma and is responsible for e.g., toxin neutralization, complement activation and opsonisation. IgG purified from human plasma is used for prophylactic prevention of infections in immunodeficient patients, replacement therapy for antibody deficiencies in patients, and the treatment of conditions relating to immune deficiencies, inflammatory and autoimmune diseases and acute infections in patients. Plasma derived immunoglobulin has become a major plasma product and world-wide consumption is increasing. Human immunoglobulin products, both hyperimmune (or ‘specific’) and normal (or ‘nonspecific’), predominantly consist of IgG. Hyperimmune immunoglobulin products include hepatitis B, tetanus, varicella-zoster and rabies immunoglobulins; each containing a known concentration of particular antibodies. The antibody specificities in normal polyvalent human immunoglobulins (IG) mirror those in the donor population. A list of FDA Approved IGs is provided at https://www.fda.gov/vaccines-blood-biologics/approved-blood-products/immune-globulins. There are currently several commercial intravenous IG (IVIG) products (typically 5% or 10% (w/v) stabilised solutions) available including Privigen® (CSL Behring), Flebogamma® (Grifols), Gamunex®-C(Grifols), Gammagard® (Takeda), and Octagam® (Octapharma). More recently, subcutaneous IG (SCIG) administration has become available. Commercial SCIG products (typically 10%, 16.5% or 20% (w/v) stabilised solutions) include Hizentra® (CSL Behring), Gamunex®-C(Grifols), Xembify® (Grifols), Cutaquig® (Octapharma) and Cuvitru® (Takeda). Other IG products are administered intramuscularly (IMIG).

IG products primarily contain IgG with a defined distribution of IgG subclasses: IgG1, IgG2, IgG3 and IgG4. IG products can however vary in different respects: IgG monomer, dimer, and aggregate concentrations; IgA and IgM content; stabilizers; additives; and levels of impurities (such as proteases like Factor XI/XIa). In regard to IgA, it is recognized that it may cause anaphylactic reactions in IgA deficient patients. For this reason, it is desirable for IG products to contain low amounts of IgA. Attributes of IG products containing IgG must also meet local and/or regional Pharmacopoeia requirements to be registered in the respective jurisdiction (e.g. Human Normal Immunoglobulin for Subcutaneous Administration, Ph. Eur. monograph 2788).

Existing methods of purifying IgG from plasma and fractions thereof include chromatography (e.g. affinity chromatography, anion exchange chromatography, hydrophobic interaction chromatography, SE-HPLC) and non-chromatography (precipitation and liquid extraction) purification methods. Major obstacles of existing methods are the high cost and time involved in purification of IgG, the requirement to co-purify other proteins from the same plasma or plasma fractions (e.g. albumin & coagulation factors) and the need to ensure that the product is of a suitable quality (e.g. purity and stability) for therapeutic use. For example, affinity resins used in affinity chromatography can have relatively low binding capacity and chromatography purification from an average size batch can reach volumes of several hundred litres (in contrast plasma fractions are typically in the thousands of litres), being a huge capital investment in the amount of resin used, the infrastructure to handle and pack the chromatography columns, along with the running costs. At present, up to 70-75% of the IgG present in plasma may be recovered from plasma using existing technologies.

It will therefore be apparent to the skilled person that there is a need in the art for improved methods of purifying IgG from plasma or fractions thereof.

### SUMMARY

The present disclosure is based on the inventors' identification of a method of purifying IgG from plasma or a fraction thereof at high yields (e.g., ≥75%). The method also allows for IgG to be recovered from plasma or a fraction thereof at high purity (e.g., ≥95%). In particular, the inventors found that the use of continuous affinity chromatography (e.g., simulated moving bed (SMB) chromatography) with an affinity chromatography resin comprising a ligand capable of specifically binding to a CH3 domain of human IgG, resulted in purification of high yields and purity of IgG from plasma with minimal impact on IgG subclass distribution (i.e. IgG1, IgG2, IgG3 and IgG4), compared to existing products. Additionally, the inventors found that the method is further improved with the use of certain wash and regeneration buffers. The method advantageously enables smaller volumes of chromatography buffers to be used and affinity resins to be reused multiple times (at least 50 cycles) further reducing the cost of purifying IgG from plasma or a fraction thereof.

Accordingly, the findings by the inventors provide the basis for a method of producing an IgG enriched preparation. The findings also provide the basis for a pharmaceutical composition comprising an IgG enriched preparation, as well as the use of the composition or IgG for treating, preventing and/or delaying progression of a condition (e.g., primary immunodeficiency disease, chronic inflammatory demyelinating polyneuropathy, and chronic immune thrombocytopenic purpura) in a subject.

The present disclosure provides an affinity chromatography resin comprising a ligand capable of specifically binding to a CH3 domain of human IgG.

The present disclosure also provides a method of purifying IgG from plasma or a fraction thereof using affinity chromatography, the method comprising binding the IgG to an affinity chromatography resin comprising a ligand capable of specifically binding to a CH3 domain of human IgG, and collecting the IgG.

The present disclosure further provides a method of producing an IgG enriched preparation from plasma or a fraction thereof using affinity chromatography, the method comprising binding the IgG to an affinity chromatography resin comprising a ligand capable of specifically binding to a CH3 domain of human IgG, and collecting the IgG.

The present disclosure provides a method of purifying IgG from plasma or a fraction thereof using continuous affinity chromatography, the method comprising binding the IgG to an affinity chromatography resin comprising a ligand capable of specifically binding to a CH3 domain of human IgG, and collecting the IgG.

The present disclosure also provides a method of producing an IgG enriched preparation from plasma or a fraction thereof using continuous affinity chromatography, the method comprising binding the IgG to an affinity chromatography resin comprising a ligand capable of specifically binding to a CH3 domain of human IgG, and collecting the IgG.

In one example, the resin comprises a ligand comprising a camelid-derived single domain [VHH] antibody fragment. For example, the ligand is a VHH antibody fragment. In one example, the ligand does not comprise a CH1 domain.

In one example, the resin comprises a matrix selected from the group consisting of a cross-linked poly(styrene-divinylbenzene) matrix and an agarose-based matrix. For example, the matrix is a cross-linked poly(styrene-divinylbenzene) matrix. In another example, the matrix is an agarose-based matrix.

In one example, the resin comprises a ligand capable of specifically binding to a CH3 domain of human IgG, wherein the ligand is conjugated to a cross-linked poly(styrene-divinylbenzene) matrix. For example, the resin comprises a ligand comprising a VHH antibody fragment conjugated to a cross-linked poly(styrene-divinylbenzene) matrix.

In one example, the resin comprises a ligand capable of specifically binding to a CH3 domain of human IgG and an agarose-based matrix. For example, the resin comprises a ligand comprising a VHH antibody fragment conjugated to an agarose-based matrix.

In one example, the resin comprises a VHH antigen-binding protein comprising an amino acid sequence set forth in SEQ ID NO: 1 or a sequence comprising at least 50% amino acid identity to a sequence set forth in SEQ ID NO: 1. In one example, the VHH antigen-binding protein comprises an amino acid sequence set forth in SEQ ID NO: 1. In one example, the VHH antigen-binding protein comprises a sequence comprising at least 50% amino acid identity to a sequence set forth in SEQ ID NO: 1.

In one example, the resin comprises a VHH antigen-binding protein comprising a framework region comprising an amino acid sequence set forth in SEQ ID NO: 1 or a sequence comprising at least 50% amino acid identity to a sequence set forth in SEQ ID NO: 1. In one example, the framework region comprises an amino acid sequence set forth in SEQ ID NO: 1. In another example, the framework region comprises a sequence comprising at least 50% amino acid identity to a sequence set forth in SEQ ID NO: 1.

In one example, the resin comprises an VHH antigen-binding protein comprising an amino acid sequence that comprises 4 framework regions, FR1, FR2, FR3 and FR4, and 3 complementarity determining regions, CDR1, CDR2 and CDR3, that are operably linked in the order FR1-CDR1-FR2-CDR2-FR3-CDR3-FR4, wherein:
a) the CDR1 has an amino acid sequence selected from the group consisting of SEQ ID No: 2 or an amino acid sequence that differs from SEQ ID NO: 2 in one or two of the amino acid residues;
b) the CDR2 has an amino acid sequence having at least 80% sequence identity with an amino acid sequence of SEQ ID NO: 3; and,
c) the CDR3 has an amino acid sequence having at least 80% sequence identity with an amino acid sequence of SEQ ID NO: 4; and, wherein each of the framework regions has at least 50% amino acid identity with the framework amino acid sequence of any one of SEQ ID NO: 1; and
   wherein each of the framework regions has at least 50% amino acid identity with the framework amino acid sequence of SEQ ID NO: 1.

In one example, the resin comprises a VHH antigen-binding protein comprising an amino acid sequence that comprises 4 framework regions, FR1, FR2, FR3 and FR4, and 3 complementarity determining regions, CDR1, CDR2 and CDR3, that are operably linked in the order FR1-CDR1-FR2-CDR2-FR3-CDR3-FR4, wherein:
a) the CDR1 has an amino acid sequence selected from the group consisting of SEQ ID No: 2 or an amino acid sequence that differs from SEQ ID NO: 2 in one or two of the amino acid residues;
b) the CDR2 has an amino acid sequence having at least 80% sequence identity with an amino acid sequence of SEQ ID NO: 3; and,
c) the CDR3 has an amino acid sequence having at least 80% sequence identity with an amino acid sequence of SEQ ID NO: 4; and, wherein each of the framework regions has at least 50% amino acid identity with the framework amino acid sequence of any one of SEQ ID NO: 1, and
   wherein each of the framework regions has at least 50% amino acid identity with the framework amino acid sequence of SEQ ID NO: 1 and wherein the antigen binding protein specifically binds to the Fc domain of a human IgG molecule and does not bind to an IgG molecule of murine origin or bovine origin.

In one example, the resin comprises a VHH antigen-binding protein comprising a CDR1 comprising an amino acid sequence selected from the group consisting of SEQ ID No: 2 or an amino acid sequence that differs from SEQ ID NO: 2 in one or two of the amino acid residues.

In one example, the resin comprises a VHH antigen-binding protein comprising a CDR2 comprising an amino acid sequence having at least 80% sequence identity with an amino acid sequence of SEQ ID NO: 3.

In one example, the resin comprises a VHH antigen-binding protein comprising a CDR3 comprising an amino acid sequence having at least 80% sequence identity with an amino acid sequence of SEQ ID NO: 4.

In one example, the method further comprises washing the resin with a wash buffer. For example, the method comprises washing the resin with a wash buffer before collecting the IgG. For example, the method comprises washing the resin with a wash buffer before collecting the bound IgG.

In one example, the method further comprises washing the resin with a wash buffer as part of collecting the IgG. For example, the method further comprises washing the resin with a wash buffer as part of collecting the bound IgG. In such a method, the washing may remove unbound or weakly bound IgG from the resin. Such unbound or weakly bound IgG may be discarded prior to collecting the bound IgG. Alternatively, the unbound or weakly bound IgG is collected. In one example, the bound, unbound and weakly bound IgG is collected. In one example, the bound and weakly bound IgG is collected. In another example, the unbound and weakly bound IgG is not collected.

In one example, the method comprises washing the resin with a wash buffer prior to collecting the IgG. For example, the method comprises washing impurities from the resin with a wash buffer and collecting the IgG. In one example, the method comprises washing the resin with a wash buffer prior to collecting the IgG and collecting the flow through. In one example, the flow through comprises the impurities. In one example, the method comprises washing the resin with a wash buffer prior to collecting the IgG and collecting the impurities in the flow through. For example, the method comprises collecting the impurities from the resin with a wash buffer. In one example, the impurities and IgG are collected. For example, the impurities and IgG are collected together. In another example, the impurities and IgG are collected separately.

In one example, the method comprises collecting a wash fraction. For example, the method comprises collecting a wash fraction prior to collecting the IgG. In one example, the wash fraction comprises the impurities. In one example, the wash fraction comprises the IgG. For example, the wash fraction comprises the unbound IgG. In one example, the wash fraction comprises the weakly bound IgG. In another example, the wash fraction comprises the unbound and weakly bound IgG. In one example, the wash fraction comprises the impurities and the IgG.

In one example, the impurities comprise albumin (α-globulins and/or β-globulins), plasma lipids, plasma proteins, proteases (e.g. serine proteases, kallikrein, plasmin and FXa), serine protease inhibitors (e.g. C1 inhibitor, alpha-1-antitrypsin and anti-thrombin) IgA and IgM, factor VIII, fibrinogen, von Willebrand factor, activated clotting factors (e.g. FXa, FIXa, FVIIa and thrombin), factor XIII, contact system factors (e.g. FXIa, FXIIa and plasma kallikrein), PKA, a factor IX, a prothrombin complex, a C1 esterase inhibitor, a protein C, an anti-thrombin III, a RhD immunoglobulin and/or platelet membrane microparticles.

In one example, a plasma protein product is produced using a method described herein.

In one example, the plasma protein product is an IgG-enriched preparation. In another example, the plasma protein product comprises purified IgG.

In one example, the plasma protein product is produced using the bound, unbound and/or weakly bound IgG. For example, the bound, unbound and/or weakly bound IgG is used to produce the plasma protein product. In one example, the plasma protein product is produced using the bound IgG. For example, the bound IgG is used to produce the plasma protein product. In one example, the plasma protein product is produced using the unbound IgG. For example, the unbound IgG is used to produce the plasma protein product. In one example, the plasma protein product is produced using the weakly bound IgG. For example, the weakly bound IgG is used to produce the plasma protein product. In one example, the plasma protein product is produced using the bound and weakly bound IgG. For example, the bound and weakly bound IgG is used to produce the plasma protein product. In one example, the plasma protein product is produced using the unbound and weakly bound IgG. For example, the unbound and weakly bound IgG is used to produce the plasma protein product. In one example, the plasma protein product is produced using the bound and unbound IgG. For example, the bound and unbound IgG is used to produce the plasma protein product. In one example, the plasma protein product is produced using the bound, unbound and weakly bound IgG. For example, the bound, unbound and weakly bound IgG is used to produce the plasma protein product.

In one example, the plasma protein product is produced using the impurities. For example, the impurities are collected and used to produce the plasma protein product.

In one example, the plasma protein product is selected from a group consisting of an albumin, a serine protease, a plasmin, a FXa, an alpha-1-antitrypsin, an IgA, an IgM, a factor VIII, a fibrinogen, a von Willebrand factor, an activated clotting factor, factor XIII, a contact system factor, a PKA, a factor IX, a prothrombin complex, a C1 esterase inhibitor, a protein C, an anti-thrombin III, a RhD immunoglobulin protein product.

In one example, the activated clotting factor is selected from a group consisting of FXa, FIXa, FVIIa and thrombin. For example, the activated clotting factor is FXa. For example, the activated clotting factor is FIXa. For example, the activated clotting factor is FVIIa. For example, the activated clotting factor is thrombin.

In one example, the contact system factor protein is selected from a group consisting of FXa, FXIIa and kallikrein. For example, the contact system factor protein is FXIa. For example, the contact system factor protein is FXII. For example, the contact system factor protein is kallikrein.

In one example, the plasma protein product is an albumin protein product. In one example, the plasma protein product is a serine protease protein product. In one example, the plasma protein product is a plasmin protein product. In one example, the plasma protein product is a FXa protein product. In one example, the plasma protein product is an alpha-1-antitrypsin protein product. In one example, the plasma protein product is an IgA protein product. In one example, the plasma protein product is an IgM protein product. In one example, the plasma protein product is a factor VIII protein product. In one example, the plasma protein product is a fibrinogen protein product. In one example, the plasma protein product is a von Willebrand factor protein product. In one example, the plasma protein product is an activated clotting factor protein product. For example, the plasma protein product is a FXa protein product. For example, the plasma protein product is a FIXa protein product. For example, the plasma protein product is a FVIIa protein product. For example, the plasma protein product is a thrombin protein product.

In one example, the plasma protein product is factor XIII protein product. In one example, the plasma protein product is a contact system factor protein product. For example, the plasma protein product is a FXIa protein product. For example, the plasma protein product is a FXII protein product. For example, the plasma protein product is a kallikrein plasma product. In one example, the plasma protein product is a PKA protein product. In one example, the plasma protein product is a factor IX protein product. In one example, the plasma protein product is a prothrombin complex protein product. In one example, the plasma protein product is a C1 esterase inhibitor protein product. In one example, the plasma protein product is a protein C protein product. In one example, the plasma protein product is an anti-thrombin III protein product. In one example, the plasma protein product is a RhD immunoglobulin protein product.

In one example, the wash buffer has a pH of between 5 and 9 and a dissociation constant (pKa) between 6.8 and 8.5 at 25° C. In one example, the wash buffer has a pH of between 5 and 10 and a dissociation constant (pKa) between 6.8 and 8.5 at 25° C.

In one example, the wash buffer has a pH of between 5 and 10. In one example, the wash buffer has a pH of between 5 and 9. For example, the wash buffer is at a pH of 5, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7, or 7.1, or 7.2, or 7.3, or 7.4, or 7.5, or 7.6 or 7.7 or 7.8, or 7.9, or 8.0, or 8.1, or 8.2, or 8.3, or 8.4, or 8.5, or 8.6, or 8.7, or 8.8, or 8.9, or 9.0, or 9.1 or 9.2, or 9.3, or 9.4, or 9.5, or 9.6, or 9.7, or 9.8, or 9.9, or 10.0.

In one example, the wash buffer has a pH of between 7 and 10 and a dissociation constant (pKa) between 6.8 and 8.5 at 25° C.

In one example, the wash buffer has a pH of between 7 and 8 and a dissociation constant (pKa) between 6.8 and 8.5 at 25° C.

In one example, the wash buffer has a pH of between 7 and 8. For example, the wash buffer has a pH of 7, or 7.1, or 7.2, or 7.3, or 7.4, or 7.5, or 7.6 or 7.7 or 7.8, or 7.9 or 8.0. In one example, the wash buffer has a pH of 7.4.

In one example, the wash buffer has a pH of between 7.4 and 7.8. For example, the wash buffer has a pH of 7.4, or 7.5, or 7.6 or 7.7 or 7.8.

In one example, the wash buffer has a pKa of between 6.8 and 8.5 at 25° C. For example, the wash buffer has a pKa of 6.8, or 6.9, or 7.0, or 7.1, or 7.2, or 7.3, or 7.4, or 7.5, or 7.6, or 7.7, or 7.8, or 7.9, or 8.0, or 8.1, or 8.2, or 8.3, or 8.4, or 8.5 at 25° C.

In one example, the wash buffer has a pKa of 7.21 at 25° C.

In one example, the wash buffer has a pH of 7.4 and dissociation constant (pKa) of 7.21 at 25° C.

In one example, the wash buffer comprises a buffering agent selected from a group consisting of sodium dihydrogen phosphate, sodium citrate, imidazole, Tris, glycylglycine, 3-morpholinopropane-1-sulfonic acid (MOPS), piperazine-N,N′-bis(2-ethanesulfonic acid) (PIPES), 2-[(2-Hydroxy-1,1-bis(hydroxymethyl)ethyl)amino]ethanesulfonic acid (TES), bis[(2-hydroxyethyl)amino]acetic acid (Bicine), 4-(2-hydroxyethyl)-1-piperazineethanesulfonic acid (HEPES), sulfurous acid, 4-(2-Hydroxyethyl)-1-piperazinepropanesulfonic acid (EPPS), N-(Hydroxyethyl)piperazine-N′-2-hydroxypropanesulfonic acid (HEPPSO), 4-(N-Morpholino)butanesulfonic acid (MOBS), Piperazine-N,N′-bis(2-hydroxypropanesulfonic acid) (POPSO), N-[Tris(hydroxymethyl)methyl]-3-amino-2-hydroxypropanesulfonic acid (TAPSO), Tricine, triethanolamine (TEA) and combinations thereof. For example, the wash buffer is a sodium dihydrogen phosphate buffer. For example, the wash buffer is an imidazole buffer. In another example, the wash buffer is a Tris buffer. In a further example, the wash buffer is a glycylglycine buffer. In one example, the wash buffer is a MOPS buffer. In another example, the wash buffer is a PIPES buffer. In a further example, the wash buffer is a TES buffer. In one example, the wash buffer is a Bicine buffer. In another example, the wash buffer is a sulfurous acid buffer. In a further example, the wash buffer is an EPPS buffer. In one example, the wash buffer is a HEPPSO buffer. In another example, the wash buffer is a MOBS buffer. In a further example, the wash buffer is a POPSO buffer. In one example, the wash buffer is a TAPSO buffer. In another example, the wash buffer is a Tricine buffer. In a further example, the wash buffer is a TEA buffer. In one example, the wash buffer is a sodium citrate buffer.

In one example, the buffering agent of the wash buffer is at a concentration of between 5 mM to 200 mM. For example, the buffering agent of the wash buffer is at a concentration of between 5 mM to 10 mM, or 5 mM to 20 mM, or 5 mM to 50 mM, or 50 mM to 100 mM, or 100 mM to 150 mM, or 150 mM to 200 mM. In another example, the buffering agent of the wash buffer is at a concentration of 5 mM, or 10 mM, or 15 mM, or 20 mM, or 25 mM, or 30 mM, or 35 mM, or 40 mM, or 45 mM, or 50 mM, or 55 mM, or 60 mM, or 65 mM, or 70 mM, or 75 mM, or 80 mM, or 85 mM, or 90 mM, or 95 mM, or 100 mM, or 105 mM, or 110 mM, or 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM.

In one example, the buffering agent of the wash buffer is at a concentration of 5 mM.

In one example, the buffering agent of the wash buffer is at a concentration of 20 mM.

In one example, the buffering agent of the wash buffer is at a concentration of 50 mM.

In one example, the buffering agent of the wash buffer is at a concentration of 100 mM.

In one example, the buffering agent of the wash buffer is at a concentration of 150 mM.

In one example, the buffering agent of the wash buffer is at a concentration of 200 mM.

In one example, the wash buffer further comprises sodium chloride. For example, the wash buffer further comprises sodium chloride at a concentration of up to 1000 mM. In one example, the sodium chloride is at a concentration of between 5 mM and 50 mM. For example, the sodium chloride is at a concentration of 5 mM, or 10 mM, or 15 mM, or 20 mM, or 25 mM, or 30 mM, or 35 mM, or 40 mM, or 45 mM, or 50 mM. In another example, the sodium chloride is at a concentration of between 50 mM and 100 mM. For example, the sodium chloride is at a concentration of 55 mM, or 60 mM, or 65 mM, or 70 mM, or 75 mM, or 80 mM, or 85 mM, or 90 mM, or 95 mM, or 100 mM. In one example, the sodium chloride is at a concentration of between 100 and 200 mM. For example, the sodium chloride is at a concentration of 105 mM, or 110 mM, 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM. In another example, the sodium chloride is at a concentration of between 200 and 300 mM. For example, the sodium chloride is at a concentration of 200 mM, or 225 mM, or 250 mM, or 275 mM, or 300 mM. In a further example, the sodium chloride is at a concentration of between 300 and 400 mM. For example, the sodium chloride is at a concentration of 300 mM, or 325 mM, or 350 mM, or 375 mM, or 400 mM. In one example, the sodium chloride is at a concentration of between 400 mM and 500 mM. For example, the sodium chloride is at a concentration of 400 mM, or 425 mM, or 450 mM, or 475 mM, or 400 mM. In another example, the sodium chloride is at a concentration of between 500 mM and 1000 mM. For example, the sodium chloride is at a concentration of 500 mM, or 550 mM, or 600 mM, or 650 mM, or 700 mM, or 750 mM, or 800 mM, or 850 mM, or 900 mM, or 950 mM, or 1000 mM. In one example, the sodium chloride is at a concentration of less than 1000 mM. For example, the sodium chloride is at a concentration of 500 mM.

In one example, the wash buffer further comprises sodium chloride, wherein the sodium chloride is at a concentration of 145 mM.

In one example, the wash buffer further comprises sodium chloride, wherein the sodium chloride is at a concentration of 500 mM.

In one example, the wash buffer comprises 20 mM sodium dihydrogen phosphate, 145 mM sodium chloride and is at a pH of 7.4.

In one example, the wash buffer comprises 20 mM sodium dihydrogen phosphate, 500 mM sodium chloride and is at a pH of 7.4.

In one example, the wash buffer further comprises a divalent salt. For example, the wash buffer further comprises a divalent salt at a concentration of up to 1000 mM. In one example, the divalent salt is at a concentration of between 5 mM and 50 mM. For example, the divalent salt is at a concentration of 5 mM, or 10 mM, or 15 mM, or 20 mM, or 25 mM, or 30 mM, or 35 mM, or 40 mM, or 45 mM, or 50 mM. In another example, the divalent salt is at a concentration of between 50 mM and 100 mM. For example, the divalent salt is at a concentration of 55 mM, or 60 mM, or 65 mM, or 70 mM, or 75 mM, or 80 mM, or 85 mM, or 90 mM, or 95 mM, or 100 mM. In one example, the divalent salt is at a concentration of between 100 and 200 mM. For example, the divalent salt is at a concentration of 105 mM, or 110 mM, 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM. In another example, the divalent salt is at a concentration of between 200 and 300 mM. For example, the divalent salt is at a concentration of 200 mM, or 225 mM, or 250 mM, or 275 mM, or 300 mM. In a further example, the divalent salt is at a concentration of between 300 and 400 mM. For example, the divalent salt is at a concentration of 300 mM, or 325 mM, or 350 mM, or 375 mM, or 400 mM. In one example, the divalent salt is at a concentration of between 400 mM and 500 mM. For example, the divalent salt is at a concentration of 400 mM, or 425 mM, or 450 mM, or 475 mM, or 400 mM. In another example, the divalent salt is at a concentration of between 500 mM and 1000 mM. For example, the divalent salt is at a concentration of 500 mM, or 550 mM, or 600 mM, or 650 mM, or 700 mM, or 750 mM, or 800 mM, or 850 mM, or 900 mM, or 950 mM, or 1000 mM. In one example, the divalent salt is at a concentration of 500 mM. In one example, the divalent salt is at a concentration of less than 1000 mM.

In one example, the wash buffer comprises sodium chloride and/or a divalent salt at a concentration of up to 1000 mM. For example, the wash buffer comprises sodium chloride and/or a divalent salt at a concentration of about 500 mM.

In one example, the divalent salt is selected from a group consisting of magnesium chloride, calcium chloride, barium chloride, copper (II) chloride, nickel chloride, manganese chloride, and a combination thereof. For example, the divalent salt is magnesium chloride. In one example, the divalent salt is calcium chloride. In another example, the divalent salt is barium chloride. In a further example, the divalent salt is copper chloride. In one example, the divalent salt is nickel chloride. In another example, the divalent salt is manganese chloride.

In one example, the method comprises collecting the IgG by eluting the IgG from the resin with an elution buffer. For example, the method comprises collecting the bound IgG by eluting the bound IgG from the resin with an elution buffer.

In one example, the elution buffer has a pH of between 3 and 5. For example, the elution buffer has a pH of 3, or 3.1, or 3.2, or 3.3, or 3.4, or 3.5, or 3.6, or 3.7, or 3.8, or 3.9, or 4, or 4.1, or 4.2, or 4.3, or 4.4, or 4.5, or 4.6, or 4.7, or 4.8, or 4.9, or 5.

In one example, the elution buffer has a pH of 4.

In one example, the elution buffer comprises a buffering agent selected from the group consisting of sodium acetate, acetic acid and sodium citrate. In one example, the elution buffer comprises sodium acetate, acetic acid, sodium citrate and sodium dihydrogen phosphate. In one example, the elution buffer is or comprises a sodium phosphate buffer and/or an acetate buffer. For example, the elution buffer comprises sodium acetate. For example, the elution buffer comprises acetic acid. For example, the elution buffer comprises sodium citrate. For example, the elution buffer comprises sodium dihydrogen phosphate.

In one example, the buffering agent of the elution buffer is at a concentration of between 5 mM to 200 mM. For example, the buffering agent of the elution buffer is at a concentration of between 5 mM to 10 mM, or 5 mM to 20 mM, or 5 mM to 50 mM, or 50 mM to 100 mM, or 100 mM to 150 mM, or 150 mM to 200 mM. For example, the buffering agent of the wash buffer is at a concentration of 5 mM, or 10 mM, or 15 mM, or 20 mM, or 25 mM, or 30 mM, or 35 mM, or 40 mM, or 45 mM, or 50 mM, or 55 mM, or 60 mM, or 65 mM, or 70 mM, or 75 mM, or 80 mM, or 85 mM, or 90 mM, or 95 mM, or 100 mM, or 105 mM, or 110 mM, or 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM.

In one example, the buffering agent of the elution buffer is at a concentration of 5 mM.

In one example, the buffering agent of the elution buffer is at a concentration of 20 mM.

In one example, the buffering agent of the elution buffer is at a concentration of 50 mM.

In one example, the buffering agent of the elution buffer is at a concentration of 100 mM.

In one example, the buffering agent of the elution buffer is at a concentration of 150 mM.

In one example, the buffering agent of the elution buffer is at a concentration of 200 mM.

In one example, the elution buffer is or comprises an acetate buffer. For example, a sodium acetate buffer.

In one example, the elution buffer is or comprises a phosphate buffer and/or an acetate buffer. For example, the elution buffer is or comprises a sodium dihydrogen phosphate and a sodium acetate buffer. In one example, the elution buffer is or comprises a phosphate buffer.

In one example, the elution buffer is or comprises an acetate buffer at a pH of between 3 and 5. For example, the elution buffer is or comprises an acetate buffer at a pH of 3, or 3.1, or 3.2, or 3.3, or 3.4, or 3.5, or 3.6, or 3.7, or 3.8, or 3.9, or 4, or 4.1, or 4.2, or 4.3, or 4.4, or 4.5, or 4.6, or 4.7, or 4.8, or 4.9, or 5. In one example, the elution buffer is or comprises an acetate buffer at a pH of 4. For example, the elution buffer is or comprises a sodium acetate buffer at a pH of 4.

In one example, the elution buffer is or comprises a phosphate and/or an acetate buffer at a pH of between 3 and 5. For example, the elution buffer is or comprises a phosphate and/or an acetate buffer at a pH of 3, or 3.1, or 3.2, or 3.3, or 3.4, or 3.5, or 3.6, or 3.7, or 3.8, or 3.9, or 4, or 4.1, or 4.2, or 4.3, or 4.4, or 4.5, or 4.6, or 4.7, or 4.8, or 4.9, or 5. In one example, the elution buffer is or comprises a phosphate and/or an acetate buffer at a pH of 4.

In one example, the elution buffer is or comprises a phosphate buffer at a pH of between 3 and 5. For example, the elution buffer is or comprises a phosphate buffer at a pH of 3, or 3.1, or 3.2, or 3.3, or 3.4, or 3.5, or 3.6, or 3.7, or 3.8, or 3.9, or 4, or 4.1, or 4.2, or 4.3, or 4.4, or 4.5, or 4.6, or 4.7, or 4.8, or 4.9, or 5. In one example, the elution buffer is or comprises a phosphate buffer at a pH of 4.

In one example, the elution buffer comprises 10 mM, or 11 mM, or 12 mM, or 13 mM, or 14 mM, or 15 mM, or 16 mM, or 17 mM, or 18 mM, or 19 mM, or 20 mM of a phosphate and/or an acetate buffer.

In one example, the elution buffer comprises 10 mM, or 11 mM, or 12 mM, or 13 mM, or 14 mM, or 15 mM, or 16 mM, or 17 mM, or 18 mM, or 19 mM, or 20 mM of an acetate buffer. For example, the elution buffer comprises 20 mM acetate buffer. In one example, the elution buffer comprises 20 mM sodium acetate buffer.

In one example, the elution buffer comprises 10 mM, or 11 mM, or 12 mM, or 13 mM, or 14 mM, or 15 mM, or 16 mM, or 17 mM, or 18 mM, or 19 mM, or 20 mM of a phosphate buffer. For example, the elution buffer comprises 20 mM phosphate buffer. In one example, the elution buffer comprises 20 mM sodium phosphate buffer.

In one example, the elution buffer comprises 20 mM sodium acetate at a pH of 4.

In one example, the elution buffer further comprises sodium chloride. For example, the elution buffer further comprises sodium chloride at a concentration of up to 150 mM. In one example, the sodium chloride is at a concentration of between 50 to 100 mM. For example, the sodium chloride is at a concentration of 5 mM, 10 mM, 15 mM, 20 mM, 25 mM, 30 mM, 35 mM, 40 mM, 45 mM, or 50 mM. In another example, the sodium chloride is at a concentration of between 100 to 150 mM. For example, the sodium chloride is at a concentration of 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, or 150 mM.

In one example, the elution buffer further comprises a divalent salt. For example, the elution buffer further comprises a divalent salt at a concentration of up to 150 mM. In one example, the divalent salt is at a concentration of between 50 to 100 mM. For example, the divalent salt is at a concentration of 5 mM, 10 mM, 15 mM, 20 mM, 25 mM, 30 mM, 35 mM, 40 mM, 45 mM, or 50 mM. In another example, the divalent salt is at a concentration of between 100 to 150 mM. For example, the divalent salt is at a concentration of 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, or 150 mM.

In one example, the divalent salt is selected from a group consisting of magnesium chloride, calcium chloride, barium chloride, copper (II) chloride, nickel chloride, manganese chloride, and a combination thereof. For example, the divalent salt is magnesium chloride. For example, the divalent salt is calcium chloride. In one example, the divalent salt is barium chloride. In another example, the divalent salt is copper chloride. In a further example, the divalent salt is nickel chloride. In one example, the divalent salt is manganese chloride.

In one example, the method further comprises equilibrating the resin with an equilibration buffer. For example, the resin is equilibrated before loading the plasma or a fraction thereof comprising IgG on to the resin.

In one example, the method further comprises equilibrating the resin with an equilibration buffer having a pH of between 5 and 9. For example, the equilibration buffer has a pH of 5, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7, or 7.1, or 7.2, or 7.3, or 7.4, or 7.5, or 7.6 or 7.7 or 7.8, or 7.9, or 8.0, or 8.1, or 8.2, or 8.3, or 8.4, or 8.5, or 8.6, or 8.7, or 8.8, or 8.9, or 9.0.

In one example, the method further comprises equilibrating the resin with an equilibration buffer having a pH between 7 and 8. For example, the equilibration buffer is at a pH of 7, or 7.1, or 7.2, or 7.3, or 7.4, or 7.5, or 7.6, or 7.7, or 7.8, or 7.9, or 8. In one example, the equilibration buffer is at a pH of 7.4.

In one example, the equilibration buffer comprises a buffering agent selected from a group consisting of sodium dihydrogen phosphate, sodium citrate, imidazole, Tris, glycylglycine, MOPS, PIPES, TES, Bicine, HEPES, EPPS, HEPPSO, MOBS, POPSO, TAPSO, Tricine, TEA and combinations thereof. For example, the equilibration buffer is a sodium dihydrogen phosphate buffer. In another example, the equilibration buffer is a sodium citrate buffer. In a further example, the equilibration buffer is an imidazole buffer. In one example, the equilibration buffer is a Tris buffer. In another example, the equilibration buffer is a glycylglycine buffer. In a further example, the equilibration buffer is a MOPS buffer. In one example, the equilibration buffer is a PIPES buffer. In another example, the equilibration buffer is a TES buffer. In a further example, the equilibration buffer is a Bicine buffer. In one example, the equilibration buffer is a sulfurous acid buffer. In another example, the equilibration buffer is an EPPS buffer. In a further example, the equilibration buffer is a HEPPSO buffer. In one example, the equilibration buffer is a MOBS buffer. In another example, the equilibration buffer is a POPSO buffer. In a further example, the equilibration buffer is a TAPSO buffer. In one example, the equilibration buffer is a Tricine buffer. In another example, the equilibration buffer is a TEA buffer.

In one example, the buffering agent of the equilibration buffer is at a concentration of between 5 mM and 200 mM. In one example, the buffering agent of the equilibration buffer is at a concentration of between 5 mM and 50 mM, for example at a concentration of 5 mM, or 10 mM, or 15 mM, or 20 mM, or 25 mM, or 30 mM, or 35 mM, or 40 mM, or 45 mM, or 50 mM. In another example, the buffering agent of the equilibration buffer is at a concentration of between 50 mM and 100 mM, for example, 55 mM, or 60 mM, or 65 mM, or 70 mM, or 75 mM, or 80 mM, or 85 mM, or 90 mM, or 95 mM, or 100 mM. In a further example, the equilibration buffer is at a concentration of between 100 mM and 150 mM, for example 105 mM, or 110 mM, or 115 mM, or 120 mM, or 125 mM, or 130 mM, or 135 mM, or 140 mM, or 145 mM, or 150 mM. In one example, the equilibration buffer is at a concentration of between 150 mM and 200 mM, for example 155 mM, or 160 mM, or 165 mM, or 170 mM, or 175 mM, or 180 mM, or 185 mM, or 190 mM, or 195 mM, or 200 mM.

In one example, the buffering agent of the equilibration buffer is at a concentration of 5 mM.

In one example, the buffering agent of the equilibration buffer is at a concentration of 20 mM.

In one example, the buffering agent of the equilibration buffer is at a concentration of 50 mM.

In one example, the buffering agent of the equilibration buffer is at a concentration of 100 mM.

In one example, the buffering agent of the equilibration buffer is at a concentration of 150 mM.

In one example, the buffering agent of the equilibration buffer is at a concentration of 200 mM.

In one example, the equilibration buffer further comprises sodium chloride. For example, the equilibration buffer further comprises sodium chloride at a concentration of up to 1000 mM. In one example, the sodium chloride is at a concentration of between 5 mM and 50 mM. For example, the sodium chloride is at a concentration of 5 mM, or 10 mM, or 15 mM, or 20 mM, or 25 mM, or 30 mM, or 35 mM, or 40 mM, or 45 mM, or 50 mM. In another example, the sodium chloride is at a concentration of between 50 mM and 100 mM. For example, the sodium chloride is at a concentration of 55 mM, or 60 mM, or 65 mM, or 70 mM, or 75 mM, or 80 mM, or 85 mM, or 90 mM, or 95 mM, or 100 mM. In one example, the sodium chloride is at a concentration of between 100 and 200 mM. For example, the sodium chloride is at a concentration of 105 mM, or 110 mM, 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM. In another example, the sodium chloride is at a concentration of between 200 and 300 mM. For example, the sodium chloride is at a concentration of 200 mM, or 225 mM, or 250 mM, or 275 mM, or 300 mM. In a further example, the sodium chloride is at a concentration of between 300 and 400 mM. For example, the sodium chloride is at a concentration of 300 mM, or 325 mM, or 350 mM, or 375 mM, or 400 mM. In one example, the sodium chloride is at a concentration of between 400 mM and 500 mM. For example, the sodium chloride is at a concentration of 400 mM, or 425 mM, or 450 mM, or 475 mM, or 400 mM. In another example, the sodium chloride is at a concentration of between 500 mM and 1000 mM. For example, the sodium chloride is at a concentration of 500 mM, or 550 mM, or 600 mM, or 650 mM, or 700 mM, or 750 mM, or 800 mM, or 850 mM, or 900 mM, or 950 mM, or 1000 mM. In one example, the sodium chloride is at a concentration of less than 1000 mM. For example, the sodium chloride is at a concentration of 500 mM.

In one example, the equilibration buffer further comprises sodium chloride, wherein the sodium chloride is at a concentration of 145 mM.

In one example, the equilibration buffer further comprises sodium chloride, wherein the sodium chloride is at a concentration of 500 mM.

In one example, the equilibration buffer further comprises a divalent salt. For example, the equilibration buffer further comprises a divalent salt at a concentration of up to 1000 mM. In one example, the divalent salt is at a concentration of between 5 mM and 50 mM. For example, the divalent salt is at a concentration of 5 mM, or 10 mM, or 15 mM, or 20 mM, or 25 mM, or 30 mM, or 35 mM, or 40 mM, or 45 mM, or 50 mM. In another example, the divalent salt is at a concentration of between 50 mM and 100 mM. For example, the divalent salt is at a concentration of 55 mM, or 60 mM, or 65 mM, or 70 mM, or 75 mM, or 80 mM, or 85 mM, or 90 mM, or 95 mM, or 100 mM. In one example, the divalent salt is at a concentration of between 100 and 200 mM. For example, the divalent salt is at a concentration of 105 mM, or 110 mM, 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM. In another example, the divalent salt is at a concentration of between 200 and 300 mM. For example, the divalent salt is at a concentration of 200 mM, or 225 mM, or 250 mM, or 275 mM, or 300 mM. In a further example, the divalent salt is at a concentration of between 300 and 400 mM. For example, the divalent salt is at a concentration of 300 mM, or 325 mM, or 350 mM, or 375 mM, or 400 mM. In one example, the divalent salt is at a concentration of between 400 mM and 500 mM. For example, the divalent salt is at a concentration of 400 mM, or 425 mM, or 450 mM, or 475 mM, or 400 mM. In another example, the divalent salt is at a concentration of between 500 mM and 1000 mM. For example, the divalent salt is at a concentration of 500 mM, or 550 mM, or 600 mM, or 650 mM, or 700 mM, or 750 mM, or 800 mM, or 850 mM, or 900 mM, or 950 mM, or 1000 mM. In one example, the divalent salt is at a concentration of less than 1000 mM. For example, the divalent salt is at a concentration of 500 mM.

In one example, the divalent salt is selected from a group consisting of magnesium chloride, calcium chloride, barium chloride, copper (II) chloride, nickel chloride, manganese chloride, and a combination thereof. For example, the divalent salt is magnesium chloride. In one example, the divalent salt is calcium chloride. In another example, the divalent salt is barium chloride. In a further example, the divalent salt is copper chloride. In one example, the divalent salt is nickel chloride. In another example, the divalent salt is manganese chloride.

In one example, the composition of the equilibration buffer is the same as the wash buffer. For example, the equilibration buffer comprises 20 mM sodium dihydrogen phosphate, 145 mM sodium chloride and is at a pH of 7.4. In another example, the equilibration buffer comprises 20 mM sodium dihydrogen phosphate, 500 mM sodium chloride and is at a pH of 7.4.

In one example, the resin is equilibrated i) after stripping the resin or ii) without stripping the resin. For example, the resin is equilibrated after stripping the resin. In another example, the resin is equilibrated without stripping the resin.

In one example, the method further comprises equilibrating the resin after stripping the resin with an equilibration buffer having a pH between 7 and 8.

In one example, the method optionally comprises stripping the resin with a stripping buffer after collecting the IgG from the resin. For example, the method further comprises stripping the resin with a stripping buffer after collecting the IgG from the resin. In another example, the method does not comprise stripping the resin with a stripping buffer after collecting the IgG from the resin. For example, the resin is not stripped after collecting the IgG from the resin.

In one example, the stripping buffer has a pH of between 2 to 3. For example, the stripping buffer has a pH of 2, or 2.1, or 2.2, or 2.3, 2.4, or 2.5, or 2.6, or 2.7, or 2.8, or 2.9, or 3. In one example, the stripping buffer is at a pH of 2.5.

In one example, the stripping buffer comprises a buffering agent selected from a group consisting of sodium dihydrogen phosphate, glycine and sodium citrate. For example, the stripping buffer comprises sodium dihydrogen phosphate. For example, the stripping buffer comprises glycine. For example, the stripping buffer comprises sodium citrate.

In one example, the buffering agent of the stripping buffer is at a concentration of between 10 mM to 500 mM. For example, the buffering agent of the stripping buffer is at a concentration of between 10 mM to 20 mM, or 10 mM to 50 mM, or 10 mM to 100 mM, or 10 mM to 100 mM, or 10 mM to 200 mM, or 10 mM to 300 mM, or 10 mM to 400 mM. For example, the buffering agent of the stripping buffer is at a concentration of 10 mM, or 15 mM, or 20 mM, or 25 mM, or 30 mM, or 35 mM, or 40 mM, or 45 mM, or 50 mM, or 55 mM, or 60 mM, or 65 mM, or 70 mM, or 75 mM, or 80 mM, or 85 mM, or 90 mM, or 95 mM, or 100 mM, or 105 mM, or 110 mM, or 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM, or 210 mM, or 220 mM, or 230 mM, or 240 mM, or 250 mM, or 260 mM, or 270 mM, or 280 mM, or 290 mM, or 300 mM, or 210 mM, or 220 mM, or 230 mM, or 240 mM, or 250 mM, or 260 mM, or 270 mM, or 280 mM, or 290 mM, or 300 mM, or 310 mM, or 320 mM, or 330 mM, or 340 mM, or 350 mM, or 360 mM, or 370 mM, or 380 mM, or 390 mM, or 400 mM, or 410 mM, or 420 mM, or 430 mM, or 440 mM, or 450 mM, or 460 mM, or 470 mM, or 480 mM, or 490 mM, or 500 mM.

In one example, the buffering agent of the stripping buffer is at a concentration of 5 mM.

In one example, the buffering agent of the stripping buffer is at a concentration of 20 mM.

In one example, the buffering agent of the stripping buffer is at a concentration of 50 mM.

In one example, the buffering agent of the stripping buffer is at a concentration of 100 mM.

In one example, the buffering agent of the stripping buffer is at a concentration of 150 mM.

In one example, the buffering agent of the stripping buffer is at a concentration of 200 mM.

In one example, the buffering agent of the stripping buffer is at a concentration of 250 mM.

In one example, the buffering agent of the stripping buffer is at a concentration of 300 mM.

In one example, the buffering agent of the stripping buffer is at a concentration of 350 mM.

In one example, the buffering agent of the stripping buffer is at a concentration of 400 mM.

In one example, the buffering agent of the stripping buffer is at a concentration of 450 mM.

In one example, the buffering agent of the stripping buffer is at a concentration of 500 mM.

In one example, the stripping buffer comprises 20 mM sodium dihydrogen phosphate and is at a pH of 2.5.

In one example, the stripping buffer further comprises sodium chloride. For example, the stripping buffer further comprises sodium chloride at a concentration of up to 1000 mM. In one example, the sodium chloride is at a concentration of between 5 mM and 50 mM. For example, the sodium chloride is at a concentration of 5 mM, or 10 mM, or 15 mM, or 20 mM, or 25 mM, or 30 mM, or 35 mM, or 40 mM, or 45 mM, or 50 mM. In another example, the sodium chloride is at a concentration of between 50 mM and 100 mM. For example, the sodium chloride is at a concentration of 55 mM, or 60 mM, or 65 mM, or 70 mM, or 75 mM, or 80 mM, or 85 mM, or 90 mM, or 95 mM, or 100 mM. In one example, the sodium chloride is at a concentration of between 100 and 200 mM. For example, the sodium chloride is at a concentration of 105 mM, or 110 mM, 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM. In another example, the sodium chloride is at a concentration of between 200 and 300 mM. For example, the sodium chloride is at a concentration of 200 mM, or 225 mM, or 250 mM, or 275 mM, or 300 mM. In a further example, the sodium chloride is at a concentration of between 300 and 400 mM. For example, the sodium chloride is at a concentration of 300 mM, or 325 mM, or 350 mM, or 375 mM, or 400 mM. In one example, the sodium chloride is at a concentration of between 400 mM and 500 mM. For example, the sodium chloride is at a concentration of 400 mM, or 425 mM, or 450 mM, or 475 mM, or 400 mM. In another example, the sodium chloride is at a concentration of between 500 mM and 1000 mM. For example, the sodium chloride is at a concentration of 500 mM, or 550 mM, or 600 mM, or 650 mM, or 700 mM, or 750 mM, or 800 mM, or 850 mM, or 900 mM, or 950 mM, or 1000 mM. In one example, the sodium chloride is at a concentration of less than 1000 mM.

In one example, the stripping buffer further comprises a divalent salt. For example, the stripping buffer further comprises a divalent salt at a concentration of up to 1000 mM. In one example, the divalent salt is at a concentration of between 5 mM and 50 mM. For example, the divalent salt is at a concentration of 5 mM, or 10 mM, or 15 mM, or 20 mM, or 25 mM, or 30 mM, or 35 mM, or 40 mM, or 45 mM, or 50 mM. In another example, the divalent salt is at a concentration of between 50 mM and 100 mM. For example, the divalent salt is at a concentration of 55 mM, or 60 mM, or 65 mM, or 70 mM, or 75 mM, or 80 mM, or 85 mM, or 90 mM, or 95 mM, or 100 mM. In one example, the divalent salt is at a concentration of between 100 and 200 mM. For example, the divalent salt is at a concentration of 105 mM, or 110 mM, 115 mM, or 120 mM, or 125 mM, or 130 mM, or 135 mM, or 140 mM, or 145 mM, or 150 mM, or 155 mM, or 160 mM, or 165 mM, or 170 mM, or 175 mM, or 180 mM, or 185 mM, or 190 mM, or 195 mM, or 200 mM. In another example, the divalent salt is at a concentration of between 200 and 300 mM. For example, the divalent salt is at a concentration of 200 mM, or 225 mM, or 250 mM, or 275 mM, or 300 mM. In a further example, the divalent salt is at a concentration of between 300 and 400 mM. For example, the divalent salt is at a concentration of 300 mM, or 325 mM, or 350 mM, or 375 mM, or 400 mM. In one example, the divalent salt is at a concentration of between 400 mM and 500 mM. For example, the divalent salt is at a concentration of 400 mM, or 425 mM, or 450 mM, or 475 mM, or 400 mM. In another example, the divalent salt is at a concentration of between 500 mM and 1000 mM. For example, the divalent salt is at a concentration of 500 mM, or 550 mM, or 600 mM, or 650 mM, or 700 mM, or 750 mM, or 800 mM, or 850 mM, or 900 mM, or 950 mM, or 1000 mM. In one example, the divalent salt is at a concentration of less than 1000 mM.

In one example, the divalent salt is selected from a group consisting of magnesium chloride, calcium chloride, barium chloride, copper (II) chloride, nickel chloride, manganese chloride, and a combination thereof. For example, the divalent salt is magnesium chloride. For example, the divalent salt is calcium chloride. For example, the divalent salt is barium chloride. For example, the divalent salt is copper chloride. For example, the divalent salt is nickel chloride. For example, the divalent salt is manganese chloride.

In one example, the resin is equilibrated. In one example, the resin is equilibrated after stripping the resin. In one example, the method further comprises equilibrating the resin with the equilibration buffer having a pH of between 7 and 8 after stripping the resin.

In one example, the method comprises:
a) equilibrating the resin with an equilibration buffer having a pH between 7 and 8;
b) stripping the resin with a stripping buffer having a pH of between 2 to 3 after collecting the bound IgG from the resin; and/or
c) equilibrating the resin with the equilibration buffer after the resin is stripped.

In one example, the resin is equilibrated without stripping the resin. For example, the method comprises equilibrating the resin with the equilibration buffer after collecting the bound IgG from the resin and without stripping the resin with a stripping buffer. In one example, the method comprises equilibrating the resin with the equilibration buffer having a pH of between 7 and 8 after collecting the bound IgG from the resin.

In one example, the method further comprises regenerating the resin.

In one example, the method further comprises sanitising the resin.

In one example, the method comprises loading the plasma or fraction thereof onto the affinity chromatography resin.

In one example, the plasma or fraction thereof contacts the resin for at least 0.1 minutes during loading of the plasma or fraction thereof. For example, the plasma or fraction thereof contacts the resin for at least 0.25 minutes, or 0.5 minutes, or 1 minute, or 1.5 minutes, or 2 minutes or 2.5 minutes, or 3 minutes, or 3.5 minutes, or 4 minutes, or 4.5 minutes, or 5 minutes. For example, the plasma or fraction thereof contacts the resin for 0.1 minutes, 0.25 minutes, 0.3 minutes, 0.35 minutes, 0.4 minutes, 0.45 minutes, 0.5 minutes, or 1 minute, or 1.5 minutes, or 2 minutes, or 2.5 minutes, or 3 minutes, or 3.5 minutes, or 4 minutes, or 4.5 minutes, or 5 minutes.

In one example, the plasma or fraction thereof contacts the resin for up to 5 minutes during loading of the plasma or fraction thereof.

In one example, the plasma or fraction thereof contacts the resin for between 0.25 and 5 minutes during loading of the plasma or fraction thereof. For example, during loading the plasma or fraction thereof contacts the resin for 0.25 minutes, 0.3 minutes, 0.35 minutes, 0.4 minutes, 0.45 minutes, 0.5 minutes, or 1 minute, or 1.5 minutes, or 2 minutes, or 2.5 minutes, or 3 minutes, or 3.5 minutes, or 4 minutes, or 4.5 minutes, or 5 minutes. In one example, during loading the plasma or fraction thereof contacts the resin for at least 0.25 minutes.

In one example, a buffer contacts the resin for at least 0.1 minutes during one or more non-loading phase(s) of the method.

In one example, the buffer contacts the resin for up to 5 minutes during one or more non-loading phase(s) of the continuous chromatography method.

In one example, the buffer contacts the resin between 0.1 and 5 minutes during one or more non-loading phase(s) of the continuous chromatography method. For example, the buffer contacts the resin for at least 0.1 minutes, or 0.25 minutes, or 0.5 minutes, or 1 minute, or 1.5 minutes, or 2 minutes or 2.5 minutes, or 3 minutes, or 3.5 minutes, or 4 minutes, or 4.5 minutes, or 5 minutes.

In one example, the non-loading phase is selected from the group consisting of an equilibration phase, a wash phase, an elution phase, a strip phase, a re-equilibration phase and combinations thereof.

In one example, the non-loading phase is an equilibration phase.

In one example, the non-loading phase is a wash phase.

In one example, the non-loading phase is an elution phase.

In one example, the non-loading phase is a strip phase.

In one example, the non-loading phase is a re-equilibration phase.

In one example, the buffer which contacts the resin during one or more non-loading phase(s) of the continuous chromatography method is selected from a group consisting of an equilibration buffer, a wash buffer, a stripping buffer, and a re-equilibration buffer. For example, the buffer is the equilibration buffer. In another example, the buffer is the wash buffer. In a further example, the buffer is the stripping buffer. In one example, the buffer is the re-equilibration buffer.

In one example, the equilibration buffer contacts the resin for between 0.1 and 5 minutes.

In one example, the wash buffer contacts the resin for between 0.1 and 5 minutes.

In one example, the elution buffer contacts the resin for between 0.1 and 5 minutes.

In one example, the stripping buffer contacts the resin for between 0.1 and 5 minutes.

In one example, the method comprises contacting the resin with a volume of elution buffer of less than a column volume (CV) before collecting the bound IgG from the resin. For example, the method comprises a ‘pre-elution’ phase of contacting the resin with a volume of elution buffer of less than a column volume (CV) before collecting the bound IgG from the resin. In one example, the method comprises washing the resin with a volume of elution buffer of less than a CV before collecting the bound IgG from the resin. For example, the volume of elution buffer used to wash the resin before collecting the bound IgG from the resin is up to 0.5 CV. For example, the volume of elution buffer used to wash the resin before collecting the bound IgG from the resin is between 0.5 and 1.0 CV. For example, the volume of elution buffer used to wash the resin before collecting the bound IgG from the resin is 0.1 CV, or 0.2 CV, or 0.3 CV, or 0.4 CV, or 0.5 CV, or 0.6 CV, or 0.7 CV, or 0.8 CV, or 0.9 CV.

In one example, the volume of the elution buffer is 0.1 CV.

In one example, the volume of the elution buffer is 0.2 CV.

In one example, the volume of the elution buffer is 0.3 CV.

In one example, the volume of the elution buffer is 0.4 CV.

In one example, the volume of the elution buffer is 0.5 CV.

In one example, the volume of the elution buffer is 0.6 CV.

In one example, the volume of the elution buffer is 0.7 CV.

In one example, the volume of the elution buffer is 0.8 CV.

In one example, the volume of the elution buffer is 0.9 CV.

In one example, the method comprises eluting the bound IgG from the resin after performing the step of contacting the resin with a volume of elution buffer of less than a CV.

It will be apparent to the skilled person that normally IgG is present in plasma at a concentration of between 5-15 g/L of plasma.

In one example, the plasma fraction is selected from a group consisting of cryo-rich plasma, cryo-poor plasma, Supernatant I (SN I), Cohn Fraction II (Fr II), Cohn Fraction II+III (Fr II+III), Cohn Fraction I+II+III (FrI+II+III), Kistler/Nitschmann Precipitate A (KN A), Kistler/Nitschmann Precipitate B (KN B), Kistler/Nitschmann Precipitate of Supernatant B (KN B+1), and combinations thereof. In one example, the plasma fraction is cryo-rich plasma. For example, the plasma fraction is cryo-poor plasma. For example, the plasma fraction is Supernatant I (SN I). For example, the plasma fraction is Cohn Fraction II (Fr II). For example, the plasma faction is Cohn Fraction II+III (Fr II+III). For example, the plasma fraction is Cohn Fraction I+II+III (FrI+II+III). For example, the plasma fraction is Kistler/Nitschmann Precipitate A (KN A). For example, the plasma fraction is Kistler/Nitschmann Precipitate B (KN B). For example, the plasma fraction is Kistler/Nitschmann Precipitate of Supernatant B (KN B+1).

In one example, the plasma fraction is a suspended paste. For example, the suspended paste is selected from a group consisting of Cohn Fraction II (Fr II), Cohn Fraction II+III (Fr II+III), Cohn Fraction I+II+III (FrI+II+III), Kistler/Nitschmann Precipitate A (KN A), Kistler/Nitschmann Precipitate B (KN B), Kistler/Nitschmann Precipitate of Supernatant B (KN B+1), and combinations thereof. For example, the suspended paste is a Cohn Fraction II (Fr II) paste. In one example, the suspended paste is a Cohn Fraction TT+TTT (Fr TT+TTT) paste. In another example, the suspended paste is a Cohn Fraction I+II+III (FrI+II+III) paste. In another example, the suspended paste is a Kistler/Nitschmann Precipitate A (KN A) paste. In another example, the suspended paste is a Kistler/Nitschmann Precipitate B (KN B) paste. In a further example, the suspended paste is a Kistler/Nitschmann Precipitate of Supernatant B (KN B+1) paste.

In one example, the plasma fraction is selected from the group consisting of a mammalian plasma fraction, a human plasma fraction, an equine plasma fraction, and a bovine plasma fraction.

In one example, the plasma fraction is a mammalian plasma fraction.

In one example, the plasma fraction is a human plasma fraction.

In one example, the plasma fraction is an equine plasma fraction.

In one example the plasma fraction is a bovine plasma fraction.

In one example the plasma fraction is a bovine plasma fraction comprising human polyclonal antibodies.

In one example, the plasma or fraction thereof is clarified. Methods of clarification of the plasma or fraction thereof will be apparent to the skilled person and/or described herein. For example, the plasma or fraction thereof is clarified by passing the plasma or fraction thereof through a filter. For example, a depth or membrane filter can be used. For example, the plasma or fraction thereof is passed through a combination of filters. For example, the combination may be a 1.2 and 0.45/0.22 μm membrane filter combination. For example, the plasma or fraction thereof is clarified by passing the plasma or fraction thereof through a depth filter (e.g. BECO® depth filter). In one example, the plasma or fraction thereof is clarified by passing the plasma or fraction thereof through a filter press (e.g. BECO® integra plate or compact plate) comprising one or more depth filter(s). In one example, the filter press further comprises one or more filter aid(s) (e.g. cellulose-based filter aids such as Diacel® 150). In one example, the plasma or fraction thereof is clarified by passing the plasma or fraction thereof through a lipid-specific filter (e.g. Zeta Plus™ DEL Series filter). For example, the plasma fraction is clarified Supernatant I (SN I). For example, the plasma fraction is clarified Cohn Fraction II (Fr II). For example, the plasma faction is clarified Cohn Fraction II+III (Fr II+III). For example, plasma fraction is clarified Cohn Fraction I+II+III (FrI+II+III). For example, the plasma fraction is clarified Kistler/Nitschmann Precipitate A (KN A). For example, the plasma fraction is clarified Kistler/Nitschmann Precipitate B (KN B). For example, the plasma fraction is clarified Kistler/Nitschmann Precipitate of Supernatant B (KN B+1).

In one example, the plasma is clarified cryo-rich plasma.

In one example, the plasma fraction is clarified cryo-poor plasma.

In one example, the plasma or fraction thereof is warmed to a first temperature of about 32° C. and then cooled to a second temperature of about 21° C. before the continuous affinity chromatography step. In one example, the plasma or fraction thereof is at a first temperature of about 32° C. and then at a second temperature of about 21° C. before the continuous affinity chromatography step.

In one example, the plasma or fraction thereof is at a temperature in the range of 2° C. to 35° C. before the continuous affinity chromatography step. In one example, the plasma or fraction thereof is at a temperature in the range of 2° C. to 28° C. before the continuous affinity chromatography step. For example, a temperature in the range of 10° C. to 28° C., such as 10° C., or 11° C., or 12° C., or 13° C., or 14° C., 15° C., or 16° C., or 17° C., or 18° C., or 19° C., or 20° C., or 21° C., or 22° C., or 23° C., or 24° C., or 25° C., or 26° C., or 27° C., or 28° C. For example, a temperature in the range of 10° C. to 28° C., such as 10° C., or 11° C., or 12° C., or 13° C., or 14° C., 15° C., or 16° C., or 17° C., or 18° C., or 19° C., or 20° C., or 21° C., or 22° C., or 23° C., or 24° C., or 25° C., or 26° C., or 27° C., or 28° C., or 29° C., or 30° C., or 31° C., or 32° C., or 33° C., or 34° C., or 35° C. In one example, the plasma or fraction thereof is at a temperature in the range of 2° C. to 35° C. before loading onto the continuous affinity chromatography resin. In one example, the plasma or fraction thereof is at a temperature in the range of 2° C. to 28° C. before loading onto the continuous affinity chromatography resin. For example, a temperature in the range of 10° C. to 35° C. For example, the plasma or fraction thereof is at a temperature in the range of from 30° C. to 35° C. For example, the plasma or fraction thereof is at a temperature of at least 32° C. For example, the plasma or fraction thereof is at a temperature in the range of from 32° C. to 35° C. In one example, the plasma or fraction thereof is at a temperature of 32° C. For example, a temperature in the range of 10° C. to 28° C. In one example, the plasma or fraction thereof is at a temperature in the range of from 2° C. to 25° C. For example, a temperature in the range of 10° C. to 25° C. In one example, the plasma or fraction thereof is at a temperature in the range of from 20° C. to 25° C. For example, the plasma or fraction thereof is at a temperature of 21° C. In one example, the plasma or fraction thereof is at a temperature in the range of from 2° C. to 20° C. For example, a temperature in the range of 10° C. to 20° C. In one example, the plasma or fraction thereof is at a temperature in the range of from 2° C. to 18° C. For example, a temperature in the range of 10° C. to 18° C. In one example, the plasma or fraction thereof is at a temperature in the range of from 2° C. to 15° C. For example, a temperature in the range of 10° C. to 15° C. In one example, the plasma or fraction thereof is at a temperature in the range of 2° C. to 10° C. For example, the plasma or fraction thereof is at a temperature of 2° C., or 3° C., or 4° C., or 5° C., or 6° C., or 7° C., or 8° C., or 9° C., or 10° C.

In one example, the plasma or fraction thereof is at a temperature of 2° C.

In one example, the plasma or fraction thereof is at a temperature of 10° C.

In one example, the plasma or fraction thereof is at a temperature of 18° C.

In one example, the plasma or fraction thereof is at a temperature of 21° C.

In one example, the plasma or fraction thereof is at a temperature of 28° C.

In one example, the plasma or fraction thereof is at a temperature of 32° C.

In one example, the plasma or fraction thereof is at the temperature for up to 48 hrs. For example, the plasma or fraction thereof is held at the temperature for up to 48 hrs prior to loading the plasma or fraction thereof onto the continuous affinity chromatography resin. In one example, the plasma or fraction thereof is held at the temperature for up to 2 hrs, or 4 hrs, or 6 hrs, or 8 hrs, or 10 hrs, or 12 hrs, or 14 hrs, or 16 hrs, or 18 hrs, or 20 hrs, or 22 hrs, or 24 hrs, or 26 hrs, or 28 hrs, or 30 hrs, or 32 hrs, or 34 hrs, or 36 hrs, or 38 hrs, or 40 hrs, or 42 hrs, or 44 hrs, or 46 hrs prior to loading. For example, the plasma or fraction thereof is held at the temperature for 0 to 2 hrs, 2 to 24 hrs, or 4 to 24 hrs, or 8 to 24 hrs, or 12 to 24 hrs, or 18 to 24 hrs, or 24 to 48 hrs, or 36 to 48 hrs prior to loading.

In one example, the plasma or fraction thereof is at a first temperature in the range of 30° C. to 38° C. and then at a second temperature in the range of 2° C. to 28° C. before the continuous affinity chromatography step. For example, the plasma or fraction thereof is warmed to a first temperature in the range of 30° C. to 38° C. and then cooled to a second temperature in the range of 2° C. to 28° C. before the continuous affinity chromatography step. In one example, the plasma or fraction thereof is warmed to a first temperature in the range of 30° C. to 35° C. and then cooled to a second temperature in the range of 18° C. to 25° C. before the continuous affinity chromatography step. For example, the plasma or fraction thereof is warmed to a first temperature of about 30° C., or about 31° C., or about 32° C., or about 33° C., or about 34° C., or about 35° C. In one example, the plasma or fraction thereof is cooled to a second temperature of about 18° C., or about 19° C., or about 20° C., or about 21° C., or about 22° C., or about 23° C., or about 24° C., or about 25° C.

In one example, the plasma or fraction thereof is at the first and/or second temperature for up to 48 hrs. For example, the plasma or fraction thereof is held at the first and/or second temperature for up to 48 hrs prior to loading the plasma or fraction thereof onto the continuous affinity chromatography resin. In one example, the plasma or fraction thereof is held at the first and/or second temperature for up to 2 hrs, or 4 hrs, or 6 hrs, or 8 hrs, or 10 hrs, or 12 hrs, or 14 hrs, or 16 hrs, or 18 hrs, or 20 hrs, or 22 hrs, or 24 hrs, or 26 hrs, or 28 hrs, or 30 hrs, or 32 hrs, or 34 hrs, or 36 hrs, or 38 hrs, or 40 hrs, or 42 hrs, or 44 hrs, or 46 hrs prior to loading. For example, the plasma or fraction thereof is held at the first and/or second temperature for 0 to 2 hrs, 2 to 24 hrs, or 4 to 24 hrs, or 8 to 24 hrs, or 12 to 24 hrs, or 18 to 24 hrs, or 24 to 48 hrs, or 36 to 48 hrs prior to loading.

In one example, the continuous affinity chromatography is selected from the group consisting of simulated moving bed (SMB) chromatography, periodic counter-current chromatography (PCC), continuous counter-current tangential chromatography (CCTC), and continuous counter-current spiral chromatography (CCSC).

In one example, the continuous affinity chromatography is simulated moving bed (SMB) chromatography. In another example, the continuous affinity chromatography is periodic counter-current chromatography (PCC). In a further example, the continuous affinity chromatography is continuous counter-current tangential chromatography (CCTC). In one example, the continuous affinity chromatography is continuous counter-current spiral chromatography (CCSC).

In one example, the resin is in the form of a slurry. For example, the resin comprises resin particles in the form of a slurry.

In one example, the slurry is passed through one or more columns wherein each column comprises a membrane. For example, the membrane is a hollow fiber membrane.

In one example, the slurry is passed through a series of two or more columns comprising the membrane. For example, the slurry is passed through two, or three, or four, or five, or six, or seven, or eight, or nine, or ten, or eleven, or twelve columns.

In one example, the slurry is passed through a series of two columns.

In one example, the slurry is passed through a series of three columns.

In one example, the slurry is passed through a series of four columns.

In one example, the slurry is passed through a series of five columns.

In one example, the slurry is passed through a series of six columns.

In one example, the slurry is passed through a series of seven columns.

In one example, the slurry is passed through a series of eight columns.

In one example, the slurry is passed through a series of nine columns.

In one example, the slurry is passed through a series of ten columns.

In one example, the slurry is passed through a series of eleven columns.

In one example, the slurry is passed through a series of twelve columns.

In one example, the resin is packed into one or more columns wherein each column comprises one or more zones. For example, the resin is packed into a series of two or more columns. For example, the resin is packed into a series of two, or three, or four, or five, or six, or seven, or eight, or nine, or ten, or eleven, or twelve columns.

In one example, the resin is packed into a series of two columns.

In one example, the resin is packed into a series of three columns.

In one example, the resin is packed into a series of four columns.

In one example, the resin is packed into a series of five columns.

In one example, the resin is packed into a series of six columns.

In one example, the resin is packed into a series of seven columns.

In one example, the resin is packed into a series of eight columns.

In one example, the resin is packed into a series of nine columns.

In one example, the resin is packed into a series of ten columns.

In one example, the resin is packed into a series of eleven columns.

In one example, the resin is packed into a series of twelve columns.

For example, a zone is selected from the group consisting of an equilibration zone, a binding zone, a wash zone, an elution zone, a stripping zone, and a combination thereof. In another example, a zone is selected from the group consisting of an equilibration zone, a binding zone, a wash zone, an elution zone, and a combination thereof. In one example, the zone is an equilibration zone. In another example, the zone is a binding zone. In a further example, the zone is a wash zone. In one example, the zone is an elution zone. In another example, the zone is a stripping zone. In one example, there is no stripping zone. In a further example, the zone is a wash/elution zone. In one example, the zone is an equilibration/binding zone. In another example, the zone is a binding/wash zone.

In one example, the resin is packed into one or more column(s), wherein each column comprises one zone.

In one example, the resin is packed into one or more column(s), wherein each column comprises two zones.

In one example, the resin is packed into one or more column(s), wherein each column comprises four zones.

In one example, the two or more columns are fluidly connected and separated by fluid conduits comprising inlet and outlet valves.

In one example, the resin is packed into a first column and one or more subsequent column(s).

In one example, the first column is loaded with IgG at a concentration above the dynamic binding capacity (DBC) of the resin.

Determining the DBC of a resin will be apparent to a skilled person and/or described herein. For example, the DBC of a resin may be determined by loading IgG on the column and monitoring the concentration at which unbound IgG flows through the column e.g. by UV trace of the chromatography system. For example, the DBC of the resin is 5 mg, or 10 mg, or 20 mg, or 30 mg, or 40 mg, or 50 mg, or 60 mg, or 70 mg IgG per mL of resin.

In one example, the DBC of the resin is at least 5 mg IgG per mL of resin.

In one example, the DBC of the resin is at least 10 mg IgG per mL of resin.

In one example, the DBC of the resin is at least 20 mg IgG per mL of resin.

In one example, the DBC of the resin is 40 mg IgG per mL of resin.

In one example, the first column is loaded with IgG at a concentration of more than 5 mg, or 10 mg, or 20 mg, or 30 mg, or 40 mg, or 50 mg, or 60 mg, or 70 mg IgG per mL of resin.

In one example, the first column is loaded with IgG at a concentration up to the DBC of the resin. For example, the first column is loaded with IgG at a concentration of up to 5 mg, or 10 mg, or 20 mg, or 30 mg, or 40 mg IgG per mL of resin.

In one example, the first column is loaded with IgG at a concentration of more than 5 mg IgG per mL of resin.

In one example, the first column is loaded with IgG at a concentration of more than 10 mg IgG per mL of resin.

In one example, the first column is loaded with IgG at a concentration of more than 20 mg IgG per mL of resin.

In one example, the first column is loaded with IgG at a concentration of up to 40 mg IgG per mL of resin.

In one example, the one or more subsequent column(s) are loaded with IgG at a concentration up to the DBC of the resin.

In one example, the one or more subsequent column(s) are loaded with IgG at a concentration of up to 5 mg, or 10 mg, or 20 mg, or 30 mg, or 40 mg IgG per mL of resin.

In one example, the one or more subsequent column(s) are loaded with IgG at a concentration of up to 20 mg IgG per mL of resin.

In one example, the one or more subsequent column(s) are loaded with IgG at a concentration of up to 30 mg IgG per mL of resin.

In one example, the one or more subsequent column(s) are loaded with IgG at a concentration of up to 40 mg IgG per mL of resin.

In one example, the method further comprises washing unbound IgG from the first column to the one or more subsequent column(s) with a wash buffer, and collecting the bound IgG. For example, the bound IgG is collected from the first and one or more subsequent column(s). For example, the bound IgG is collected from the first column without a washing step. For example, the bound IgG is collected from the first column by eluting the bound IgG with an elution buffer described herein. For example, the bound IgG is collected from one or more subsequent column(s) following washing with a wash buffer described herein. For example, the bound IgG is collected from one or more subsequent column(s) following washing the resin with a wash buffer and eluting the bound IgG with an elution buffer described herein.

In one example, the method further comprises washing the one or more subsequent column(s) with a wash buffer described herein and collecting the bound IgG from the one or more subsequent column(s).

In one example, the method further comprises stripping and/or equilibrating the first column at the time the bound IgG is collected from the one or more subsequent column(s). In one example, the method further comprises equilibrating the first column at the time the bound IgG is collected from the one or more subsequent column(s). For example, the method does not comprise stripping the first column at the time the bound IgG is collected from the one or more subsequent column(s).

In one example, the method further comprises stripping and/or equilibrating the one or more subsequent column(s) at the time bound IgG is collected from the first column. In one example, the method further comprises equilibrating the one or more subsequent column(s) at the time bound IgG is collected from the first column. For example, the method does not comprise stripping the one or more subsequent column(s) at the time bound IgG is collected from the first column.

In one example, the method further comprises stripping and/or equilibrating the first column at the time the one or more subsequent column(s) are washed with a wash buffer described herein. In one example, the method further comprises equilibrating the first column at the time the one or more subsequent column(s) are washed with a wash buffer described herein. For example, the method does not comprise stripping the first column at the time the one or more subsequent column(s) are washed with a wash buffer described herein.

In one example, the method further comprises stripping and/or equilibrating the one or more subsequent column(s) at the time the first column is washed with a wash buffer described herein. In one example, the method further comprises equilibrating the one or more subsequent column(s) at the time the first column is washed with a wash buffer described herein. For example, the method does not comprise stripping the one or more subsequent column(s) at the time the first column is washed with a wash buffer described herein.

In one example, the resin has a total bed height of at least 2 cm. For example, the resin has a total bed height of between 2 cm to 30 cm. For example, the resin has a total bed height of between 10 cm and 30 cm. For example, the resin has a total bed height of between 30 cm and 70 cm. For example, the resin has a total bed height of 2 cm, or 6 cm, or 10 cm, or 15 cm, or 20 cm, or 25 cm, or 30 cm, or 35 cm, or 40 cm, or 45 cm, or 50 cm, or 55 cm, or 60 cm, or 65 cm, or 70 cm.

In one example, the resin has a total bed height of at least 2 cm.

In one example, the resin has a total bed height of 6 cm.

In one example, the resin has a total bed height of 20 cm.

In one example, the resin has a total bed height of 30 cm.

In one example, the resin has a total bed height of 50 cm.

In one example, the resin has a total bed height of 70 cm.

In one example, the column has a diameter of between 5 cm and 200 cm. For example, the column has a diameter of 5 cm, or 10 cm, or 20 cm, or 30 cm, or 40 cm, or 50 cm, or 60 cm, or 70 cm, or 80 cm, or 90 cm, or 100 cm, or 110 cm, or 120 cm, or 130 cm, or 140 cm, or 150 cm, or 160 cm, or 170 cm, or 180 cm, or 190 cm, or 200 cm.

In one example, the column has a diameter of 5 cm.

In one example, the column has a diameter of 20 cm.

In one example, the column has a diameter of 50 cm.

In one example, the column has a diameter of 100 cm.

In one example, the column has a diameter of 200 cm.

In one example, the method further comprises one or more steps selected from ethanol precipitation, octanoic acid fractionation, membrane or resin chromatography (for example, ion exchange chromatography, hydrophobic interaction chromatography, isoagglutinin affinity chromatography), viral inactivation, viral filtration and ultrafiltration/diafiltration, wherein the step(s) are performed before or after the continuous affinity chromatography step. For example, the method further comprises ethanol precipitation. For example, the method further comprises octanoic acid fractionation. For example, the method further comprises membrane or resin chromatography. For example, the method further comprises ion exchange chromatography. For example, the method further comprises anion exchange chromatography. For example, the method further comprises cation exchange chromatography. For example, the method comprises hydrophobic interaction chromatography. For example, the method comprises isoagglutinin affinity chromatography. For example, the method further comprises viral inactivation. For example, the method further comprises nanofiltration. For example, the method further comprises ultrafiltration/diafiltration.

In one example, the method further comprises anion exchange chromatography and viral filtration.

In one example, the method further comprises low pH incubation, depth filtration, anion exchange chromatography and viral filtration.

In one example, the low pH incubation is performed in the presence of detergent. For example, the method further comprises low pH incubation in the presence of detergent.

In one example, the method further comprises ion exchange chromatography, wherein the ion exchange chromatography step comprises anion exchange chromatography step using an anion exchange resin operated in flow through mode.

In one example, the flowthrough and/or post-wash eluate is collected. For example, the flow through is collected. In another example, the post-wash eluate is collected. In one example, the flow through and post-wash eluate is collected. It will be apparent to the skilled person that only the flow through and post-wash are collected (i.e., pooled) and not the elution phase.

In one example, the anion exchange resin is selected from the group consisting of a weak anion exchanger, a strong anion exchanger and a mixed mode anion exchanger.

In one example, the anion exchange resin is a weak anion exchanger.

In one example, the anion exchange resin is a mixed mode anion exchanger.

In one example, the anion exchange resin is a strong anion exchanger. In one example, the ion exchange chromatography step comprises anion exchange chromatography step using a strong anion exchange resin operated in flow through mode.

In one example, the strong anion exchange resin comprises a matrix consisting of a poly(styrene-divinylbenzene) matrix. In one example, the strong anion exchange resin comprises a quaternized polyethyleneimine functional group.

In one example, the anion exchange resin is washed with a pre-equilibration buffer prior to equilibration. It will be apparent to the skilled person that the pre-equilibration step is only performed for the first run and/or after storage of the resin.

In one example, the pre-equilibration buffer is selected from the group consisting of monosodium phosphate (NaH₂PO₄), disodium phosphate (Na₂HPO₄), phosphoric acid (H₃PO₄) and combinations thereof.

In one example, the pre-equilibration buffer comprises Na₂HPO₄.

In one example, the pre-equilibration buffer comprises H₃PO₄.

In one example, the pre-equilibration buffer comprises NaH₂PO₄.

In one example, the pre-equilibration buffer comprises Na₂HPO₄ and NaH₂PO₄.

In one example, the pre-equilibration buffer comprises a buffer at a concentration in the range of 50 mM to 150 mM. For example, at a concentration of 50 mM, 60 mM, 70 mM, 80 mM, 90 mM, 100 mM, 110 mM, 120 mM, 130 mM, 140 mM, or 150 mM. In one example, the pre-equilibration buffer comprises a buffer at a concentration of 100 mM.

In one example, the pre-equilibration buffer comprises NaH₂PO₄ at a concentration in the range of 50 mM to 150 mM. For example, the pre-equilibration buffer comprises NaH₂PO₄ at a concentration of 50 mM, 60 mM, 70 mM, 80 mM, 90 mM, 100 mM, 110 mM, 120 mM, 130 mM, 140 mM, or 150 mM. In one example, the pre-equilibration buffer comprises NaH₂PO₄ at a concentration of 100 mM.

In one example, the pre-equilibration buffer is at a pH in the range of 5.8 to 6.6. For example, the pre-equilibration buffer is at a pH of about 5.8, or about 5.9, or about 6.0, or about 6.1, or about 6.2, or about 6.3, or about 6.4, or about 6.5, or about 6.6. In one example, the pre-equilibration is at a pH of 6.2.

In one example, the pre-equilibration buffer further comprises a salt. For example, the pre-equilibration buffer further comprises sodium chloride. In one example, the sodium chloride is at a concentration in the range of 100 mM to 1000 mM. For example, the pre-equilibration buffer comprises sodium chloride at a concentration of 100 mM, 200 mM, 300 mM, 400 mM, 500 mM, 600 mM, 700 mM, 800 mM, 900 mM, or 1000 mM. In one example, the pre-equilibration buffer comprises sodium chloride at a concentration of 1000 mM.

In one example, the anion exchange resin is pre-equilibrated with a pre-equilibration buffer comprising 1000 mM NaH₂PO₄, and 1000 mM sodium chloride, pH 6.2. In one example, the anion exchange resin is pre-equilibrated with a pre-equilibration buffer comprising 100 mM NaH₂PO₄, and 1000 mM sodium chloride, pH 6.2 In one example, the volume of the pre-equilibration buffer is at least 2 CVs. For example, the volume of the pre-equilibration buffer is 2 CVs, or 3 CVs, or 4 CVs, or 5 CVs, or 6 CVs, or 7 CVs, or 8 CVs, or 9 CVs, or 10 CVs. In one example, the volume of the pre-equilibration buffer is between 2 CVs and 10 CVs.

In one example, the volume of the pre-equilibration buffer is at least 10 CVs. For example, the volume of the pre-equilibration buffer is 10 CVs, or 11 CVs, or 12 CVs, or 13 CVs, or 14 CVs, or 15 CVs, or 16 CVs, or 17 CVs, or 18 CVs, or 19 CVs, or 20 CVs. In one example, the volume of the pre-equilibration buffer is 15 CVs.

In one example, the anion exchange resin is equilibrated with an equilibration buffer selected from the group consisting of monosodium phosphate (NaH₂PO₄), disodium phosphate (Na₂HPO₄), phosphoric acid (H₃PO₄), sodium citrate, 2-(N-morpholino)ethanesulfonic acid (MES), Bis-Tris, L-Histidine and combinations thereof.

In one example, the anion exchange resin is equilibrated with an equilibration buffer comprising Na₂HPO₄.

In one example, the anion exchange resin is equilibrated with an equilibration buffer comprising H₃PO₄.

In one example, the anion exchange resin is equilibrated with an equilibration buffer comprising NaH₂PO₄.

In one example, the anion exchange resin is equilibrated with an equilibration buffer comprising Na₂HPO₄ and NaH₂PO₄.

In one example, the anion exchange resin is equilibrated with an equilibration buffer comprising MES.

In one example, the anion exchange resin is equilibrated with an equilibration buffer comprising sodium citrate.

In one example, the anion exchange resin is equilibrated with an equilibration buffer comprising Bis-Tris.

In one example, the anion exchange resin is equilibrated with an equilibration buffer comprising L-histidine.

In one example, the equilibration buffer is at a concentration in the range of 5 mM to 50 mM. For example, the equilibration buffer is at a concentration of 5 mM, or 10 mM, or 20 mM, or 30 mM, or 40 mM, or 50 mM. In one example, the equilibration buffer is at a concentration of 5 mM. In another example, the equilibration buffer is at a concentration of 10 mM. In a further example, the equilibration buffer is at a concentration of 20 mM. In one example, the equilibration buffer is at a concentration of 30 mM. In another example, the equilibration buffer is at a concentration of 40 mM. In a further example, the equilibration buffer is at a concentration of 50 mM.

In one example, the equilibration buffer comprises NaH₂PO₄ at a concentration in the range of 5 mM to 50 mM. In one example, the equilibration buffer comprises NaH₂PO₄ at a concentration in the range of 10 mM to 50 mM. For example, the equilibration buffer comprises NaH₂PO₄ at a concentration of 10 mM, 20 mM, 30 mM, 40 mM, 50 mM. In one example, the equilibration buffer comprises NaH₂PO₄ at a concentration of 5 mM. In one example, the equilibration buffer comprises NaH₂PO₄ at a concentration of 10 mM.

In one example, the equilibration buffer is at a pH in the range of 5.8 to 6.6. For example, the equilibration buffer is at a pH of about 5.8, or about 5.9, or about 6.0, or about 6.1, or about 6.2, or about 6.3, or about 6.4, or about 6.5, or about 6.6. In one example, the equilibration is at a pH of 6.2.

In one example, the anion exchange resin is equilibrated with an equilibration buffer comprising a phosphate buffer at a pH in the range of 5.8 to 6.6. In one example, the equilibration buffer comprises a phosphate buffer pH 6.0. In one example, the equilibration buffer comprises a phosphate buffer pH 6.2. In one example, the equilibration buffer comprises a phosphate buffer pH 6.6. In one example, the anion exchange resin is equilibrated with an equilibration buffer comprising 5 mM NaH₂PO₄, pH 6.2. In one example, the anion exchange resin is equilibrated with an equilibration buffer comprising 10 mM NaH₂PO₄, pH 6.2.

In one example, the anion exchange resin is equilibrated with an equilibration buffer comprising a MES buffer at a pH in the range of 5.8 to 6.6. In one example, the equilibration buffer comprises MES buffer pH 6.0. In one example, the equilibration buffer comprises a MES buffer pH 6.2. In one example, the equilibration buffer comprises MES buffer pH 6.6.

In one example, the anion exchange resin is equilibrated with an equilibration buffer comprising a Bis-Tris buffer at a pH in the range of 5.8 to 6.6. In one example, the equilibration buffer comprises a Bis-Tris buffer pH 6.0. In one example, the equilibration buffer comprises a Bis-Tris buffer pH 6.2. In one example, the equilibration buffer comprises a Bis-Tris buffer pH 6.6.

In one example, the anion exchange resin is equilibrated with an equilibration buffer comprising a L-histidine buffer at a pH in the range of 5.8 to 6.6. In one example, the equilibration buffer comprises a L-histidine buffer pH 6.0. In one example, the equilibration buffer comprises a L-histidine buffer pH 6.2. In one example, the equilibration buffer comprises a L-histidine buffer pH 6.6.

In one example, the volume of the equilibration buffer is at least 2 CVs. For example, the volume of the equilibration buffer is 2 CVs, or 3 CVs, or 4 CVs, or 5 CVs, or 6 CVs, or 7 CVs, or 8 CVs, or 9 CVs, or 10 CVs. In one example, the volume of the equilibration buffer is between 2 CVs and 10 CVs.

In one example, the volume of the equilibration buffer is at least 10 CVs. For example, the volume of the equilibration buffer is 10 CVs, or 11 CVs, or 12 CVs, or 13 CVs, or 14 CVs, or 15 CVs, or 16 CVs, or 17 CVs, or 18 CVs, or 19 CVs, or 20 CVs. In one example, the volume of the equilibration buffer is 15 CVs.

In one example, the anion exchange resin is loaded with IgG at a concentration in the range of 5 g IgG per L of resin to 15 g IgG per L of resin. For example, the resin is loaded with 5 g, or 6 g, or 7 g, or 8 g, or 9 g, or 10 g, or 11 g, or 12 g, or 13 g, or 14 g, or 15 g IgG per L of resin. In one example, the resin is loaded with 15 g IgG per L of resin.

In one example, the anion exchange resin is loaded with IgG at a concentration in the range of 5 g IgG per L of load to 15 g IgG per L of load. For example, the resin is loaded with 5 g/L, or 6 g/L, or 7 g/L, or 8 g/L, or 9 g/L, or 10 g/L, or 11 g/L, or 12 g/L, or 13 g/L, or 14 g/L, or 15 g/L IgG. In one example, the resin is loaded with 15 g IgG per L of load.

In one example, the anion exchange chromatography step comprises a post-load wash buffer selected from the group consisting of a phosphate buffer, a sodium citrate buffer, a 2-(N-morpholino)ethanesulfonic acid buffer, an acetic acid buffer, a Bis-tris buffer and a L-histidine buffer. In one example, the anion exchange chromatography step comprises a post-load wash buffer selected from the group consisting of a phosphate buffer, a sodium citrate buffer and an acetic acid buffer.

In one example, the post-load wash buffer is at a concentration in the range of 5 mM to 50 mM. In one example, the post-load wash buffer is at a concentration in the range of 10 mM to 50 mM. For example, the post-load wash buffer is at a concentration of 10 mM, 20 mM, 30 mM, 40 mM, 50 mM. In one example, the post-load wash buffer is at a concentration of 5 mM. In one example, the post-load wash buffer is at a concentration of 10 mM.

In one example, the post-load wash buffer comprises a phosphate buffer. For example, the phosphate buffer is selected from the group consisting of monosodium phosphate (NaH₂PO₄), disodium phosphate (Na₂HPO₄), phosphoric acid (H₃PO₄) and combinations thereof.

In one example, the post-load wash buffer comprises Na₂HPO₄.

In one example, the post-load wash buffer comprises H₃PO₄.

In one example, the post-load wash buffer comprises NaH₂PO₄. For example, the post-load wash buffer comprises 5 mM NaH₂PO₄. In another example, the post-load wash buffer comprises 10 mM NaH₂PO₄.

In one example, the post-load wash buffer comprises Na₂HPO₄ and NaH₂PO₄.

In one example, the post-load wash buffer comprises a sodium citrate buffer.

In one example, the post-load wash buffer comprises an acetic acid buffer. For example, the post-load wash buffer comprises sodium acetate. For example, the post-load wash buffer comprises 5 mM acetic acid. In another example, the post-load wash buffer comprises 10 mM acetic acid.

In one example, the post-load wash buffer comprises a phosphate buffer and an acetic acid buffer. For example, the post-load wash buffer comprises NaH₂PO₄ and sodium acetate. For example, the post-load wash buffer comprises 5 mM NaH₂PO₄ and 10 mM sodium acetate.

In one example, the post-load wash buffer comprises a MES buffer.

In one example, the post-load wash buffer is a Bis-Tris buffer.

In one example, the post-load wash buffer is a L-histidine buffer.

In one example, the post-load wash buffer has a pH in the range of 5.0 to about 8.0. For example, the post-load wash buffer has a pH in the range of 5.5 to 7.0. In another example, the post-load wash buffer has a pH in the range of 5.8 to 6.6. For example, the post-load wash buffer is at a pH of about 5.8, 5.9, 6.0, 6.1, 6.2, 6.3, 6.4, 6.5 or 6.6. In one example, the post-load wash buffer is at a pH of 6.0. In one example, the post-load wash buffer is at a pH of 6.2. In another example, the post-load wash buffer is at a pH of 6.6.

In one example, the post-load wash buffer further comprises a salt. For example, the salt is sodium chloride.

In one example, the post-load wash buffer does not comprise a salt.

In one example, the sodium chloride is at a concentration of between 0 mM to 200 mM. In one example, the sodium chloride is at a concentration of 0 mM and 50 mM. In one example, the sodium chloride is at a concentration of 0 mM and 100 mM. For example, the sodium chloride is at a concentration of between 20 mM and 150 mM. In one example, the sodium chloride is at a concentration of between 20 mM and 80 mM. For example, the sodium chloride is at a concentration of about 20 mM, or 30 mM, or 40 mM, or 50 mM, or 60 mM, or 70 mM, or 80 mM. In one example, the sodium chloride is at a concentration of about 20 mM. In one example, the sodium chloride is at a concentration of about 25 mM. For example, the sodium chloride is at a concentration of 50 mM. In one example, the sodium chloride is at a concentration of about 70 mM. In one example, the sodium chloride is at a concentration of between 120 mM and 200 mM. For example, the sodium chloride is at a concentration of 150 mM. In another example, the sodium chloride is at a concentration of 200 mM.

In one example, the post-load wash buffer comprises a phosphate buffer at a pH in the range of 5.8 to 6.6.

In one example, the post-load wash buffer comprises phosphate buffer pH 6.0.

In one example, the post-load wash buffer comprises phosphate buffer pH 6.2.

In one example, the post-load wash buffer comprises phosphate buffer pH 6.6.

In one example, the post-load wash buffer comprises phosphate buffer and 0 mM sodium chloride pH 6.0.

In one example, the post-load wash buffer comprises phosphate buffer and 20 mM sodium chloride pH 6.0.

In one example, the post-load wash buffer comprises phosphate buffer and 50 mM sodium chloride pH 6.0.

In one example, the post-load wash buffer comprises phosphate buffer and 0 mM sodium chloride pH 6.6.

In one example, the post-load wash buffer comprises phosphate buffer and 25 mM sodium chloride pH 6.6.

In one example, the post-load wash buffer comprises phosphate buffer and 50 mM sodium chloride pH 6.6.

In one example, the post-load wash buffer comprises phosphate buffer and 0 mM sodium chloride pH 6.2.

In one example, the post-load wash buffer comprises a MES buffer at a pH in the range of 5.8 to 6.6.

In one example, the post-load wash buffer comprises MES buffer pH6.0.

In one example, the post-load wash buffer comprises MES buffer pH6.6.

In one example, the post-load wash buffer comprises MES buffer and 20 mM sodium chloride pH6.0.

In one example, the post-load wash buffer comprises MES buffer and 50 mM sodium chloride pH6.0.

In one example, the post-load wash buffer comprises MES buffer and 25 mM sodium chloride pH6.6.

In one example, the post-load wash buffer comprises MES buffer and 50 mM sodium chloride pH6.6.

In one example, the post-load wash buffer comprises a sodium citrate buffer at a 20 pH in the range of 5.8 to 6.6.

In one example, the post-load wash buffer comprises sodium citrate buffer pH6.0.

In one example, the post-load wash buffer comprises sodium citrate buffer pH6.6.

In one example, the post-load wash buffer comprises sodium citrate buffer and 20 mM sodium chloride pH 6.0.

In one example, the post-load wash buffer comprises sodium citrate buffer and 50 mM sodium chloride pH 6.0.

In one example, the post-load wash buffer comprises sodium citrate buffer and 25 mM sodium chloride pH 6.6.

In one example, the post-load wash buffer comprises sodium citrate buffer and 50 mM sodium chloride pH 6.6.

In one example, the post-load wash buffer comprises a sodium acetate buffer at a pH in the range of 5.8 to 6.6.

In one example, the post-load wash buffer comprises a sodium acetate buffer pH 6.0.

In one example, the post-load wash buffer comprises a sodium acetate buffer pH 6.2.

In one example, the post-load wash buffer comprises a sodium acetate buffer pH 6.6.

In one example, the post-load wash buffer comprises a sodium acetate buffer and 0 mM sodium chloride pH 6.0.

In one example, the post-load wash buffer comprises a sodium acetate buffer and 20 mM sodium chloride pH 6.0.

In one example, the post-load wash buffer comprises a sodium acetate buffer and 50 mM sodium chloride pH 6.0.

In one example, the post-load wash buffer comprises a sodium acetate buffer and 0 mM sodium chloride pH 6.6.

In one example, the post-load wash buffer comprises a sodium acetate buffer and 25 mM sodium chloride pH 6.6.

In one example, the post-load wash buffer comprises a sodium acetate buffer and 50 mM sodium chloride pH 6.6.

In one example, the post-load wash buffer comprises a sodium acetate buffer and 0 mM sodium chloride pH 6.2.

In one example, the post-load wash buffer comprises a phosphate and a sodium acetate buffer at a pH in the range of 5.8 to 6.6.

In one example, the post-load wash buffer comprises a phosphate and a sodium acetate buffer pH 6.0.

In one example, the post-load wash buffer comprises a phosphate and a sodium acetate buffer pH 6.2.

In one example, the post-load wash buffer comprises a phosphate and a sodium acetate buffer pH 6.6.

In one example, the post-load wash buffer comprises a phosphate and a sodium acetate buffer and 0 mM sodium chloride pH 6.0.

In one example, the post-load wash buffer comprises a phosphate and a sodium acetate buffer and 20 mM sodium chloride pH 6.0.

In one example, the post-load wash buffer comprises a phosphate and a sodium acetate buffer and 50 mM sodium chloride pH 6.0.

In one example, the post-load wash buffer comprises a phosphate and a sodium acetate buffer and 0 mM sodium chloride pH 6.6.

In one example, the post-load wash buffer comprises a phosphate and a sodium acetate buffer and 25 mM sodium chloride pH 6.6.

In one example, the post-load wash buffer comprises a phosphate and a sodium acetate buffer and 50 mM sodium chloride pH 6.6.

In one example, the post-load wash buffer comprises a phosphate and a sodium acetate buffer and 0 mM sodium chloride pH 6.2.

In one example, the post-load wash buffer comprises a Bis-Tris buffer at a pH in the range of 5.8 to 6.6.

In one example, the post-load wash buffer comprises Bis-Tris buffer pH6.0.

In one example, the post-load wash buffer comprises Bis-Tris buffer pH6.6.

In one example, the post-load wash buffer comprises Bis-Tris buffer and 20 mM sodium chloride pH6.0.

In one example, the post-load wash buffer comprises Bis-Tris buffer and 50 mM sodium chloride pH6.0.

In one example, the post-load wash buffer comprises Bis-Tris buffer and 25 mM sodium chloride pH6.6.

In one example, the post-load wash buffer comprises Bis-Tris buffer and 50 mM sodium chloride pH6.6.

In one example, the post-load wash buffer comprises a L-histidine buffer at a pH in the range of 5.8 to 6.6.

In one example, the post-load wash buffer comprises L-histidine buffer pH6.0.

In one example, the post-load wash buffer comprises L-histidine buffer pH6.6.

In one example, the post-load wash buffer comprises L-histidine buffer and 20 mM sodium chloride pH6.0.

In one example, the post-load wash buffer comprises L-histidine buffer and 50 mM sodium chloride pH6.0.

In one example, the post-load wash buffer comprises L-histidine buffer and 25 mM sodium chloride pH6.6.

In one example, the post-load wash buffer comprises L-histidine buffer and 50 mM sodium chloride pH6.6.

In one example, the volume of post-load wash buffer is between 1 and 5 CVs. For example, the volume of post-load wash buffer is 1 CV, or 2 CVs, or 3 CVs, or 4 CVs, or 5 CVs. In one example, the volume of post-load wash buffer is 3 CVs.

In one example, the anion exchange resin is regenerated with a regeneration buffer selected from the group consisting of sodium chloride, sodium dihydrogen phosphate, sodium hydroxide, acetic acid and combinations thereof.

In one example, the anion exchange resin is regenerated with a regeneration buffer selected from the group consisting of sodium chloride, a phosphate buffer, a sodium hydroxide buffer, an acetic acid buffer and combinations thereof.

In one example, the anion exchange resin is regenerated with a regeneration buffer comprising a phosphate buffer. For example, the phosphate buffer is selected from the group consisting of monosodium phosphate (NaH₂PO₄), disodium phosphate (Na₂HPO₄), phosphoric acid (H₃PO₄) and combinations thereof.

In one example, the regeneration buffer comprises Na₂HPO₄.

In one example, the regeneration buffer comprises H₃PO₄.

In one example, the regeneration buffer comprises NaH₂PO₄.

In one example, the regeneration buffer comprises Na₂HPO₄ and NaH₂PO₄.

In one example, the regeneration buffer comprises sodium chloride. In another example, the regeneration buffer comprises sodium hydroxide. In a further example, the regeneration buffer comprises acetic acid.

In one example, the regeneration buffer comprises sodium chloride and a phosphate buffer. In one example, the regeneration buffer comprises sodium chloride and sodium dihydrogen phosphate (NaH₂PO₄). In one example, the regeneration buffer comprises sodium chloride and Na₂HPO₄. In one example, the regeneration buffer comprises sodium chloride and H₃PO₄. In one example, the regeneration buffer comprises sodium chloride and Na₂HPO₄ and NaH₂PO₄.

In one example, the regeneration buffer comprises 1 M sodium chloride and 10 mM sodium dihydrogen phosphate, pH 6.2.

In one example, the regeneration buffer comprises 1 M sodium chloride and 10 mM Na₂HPO₄, pH 6.2.

In one example, the regeneration buffer comprises 1 M sodium chloride and 10 mM H₃PO₄, pH 6.2.

In one example, the regeneration buffer comprises 1 M sodium chloride and 10 mM Na₂HPO₄ and NaH₂PO₄, pH 6.2.

In one example, the regeneration buffer comprises 1 M sodium chloride and 100 mM sodium dihydrogen phosphate, pH 6.2.

In one example, the regeneration buffer comprises 1 M sodium chloride and 100 mM Na₂HPO₄, pH 6.2.

In one example, the regeneration buffer comprises 1 M sodium chloride and 100 mM H₃PO₄, pH 6.2.

In one example, the regeneration buffer comprises 1 M sodium chloride and 100 mM Na₂HPO₄ and NaH₂PO₄, pH 6.2.

In one example, the regeneration buffer comprises 0.5 M sodium hydroxide.

In one example, the regeneration buffer comprises 1 M acetic acid.

In one example, the volume of regeneration buffer is between 1 and 10 CVs. For example, the volume of regeneration buffer is 1 CV, or 2 CVs, or 3 CVs, or 4 CVs, or 5 CVs, or 6 CVs, or 7 CVs, or 8 CVs, or 9 CVs, or 10 CVs. In one example, the volume of regeneration buffer is 5 CVs.

Suitable regeneration methods will be apparent to the skilled person and/or described herein.

In one example, at least 75% of the IgG is recovered from the plasma or fraction thereof. In another example, at least 75% of the IgG is recovered from the plasma or fraction thereof following the continuous chromatography method. For example, at least 75% of the IgG is recovered from the plasma or fraction thereof following the continuous chromatography method without further purification steps. For example, at least 75% of the IgG is recovered from the plasma or fraction thereof following the continuous chromatography method with further purification steps. For example, at least 75% of the IgG is recovered from the plasma or fraction thereof following an ion exchange chromatography step. In one example, at least 75% of the IgG is recovered from the plasma or fraction thereof following an anion exchange chromatography step. In one example, at least 75% of the IgG is recovered from the plasma or fraction thereof, wherein the IgG is derived from at least 500 kg of plasma or fractions thereof. For example, at least 75% of the IgG is recovered from large scale purification of the plasma or fraction thereof. For example, 75%, or 76%, or 77%, or 78%, or 79% of IgG is recovered from the plasma or fraction thereof. In one example, 75% of the IgG is recovered from the plasma or fraction thereof. In one example, 76% of the IgG is recovered from the plasma or fraction thereof. In one example, 77% of the IgG is recovered from the plasma or fraction thereof. In one example, 78% of the IgG is recovered from the plasma or fraction thereof. In another example, 79% of the IgG is recovered from the plasma or fraction thereof.

In one example, at least 80% of the IgG is recovered from the plasma or fraction thereof. In another example, at least 80% of the IgG is recovered from the plasma or fraction thereof following the continuous chromatography method. For example, at least 80% of the IgG is recovered from the plasma or fraction thereof following the continuous chromatography method without further purification steps. For example, at least 80% of the IgG is recovered from the plasma or fraction thereof following the continuous chromatography method with further purification steps. For example, at least 80% of the IgG is recovered from the plasma or fraction thereof following an ion exchange chromatography step. In one example, at least 80% of the IgG is recovered from the plasma or fraction thereof following an anion exchange chromatography step. In one example, at least 80% of the IgG is recovered from the plasma or fraction thereof, wherein the IgG is derived from at least 500 kg of plasma or fractions thereof. For example, at least 80% of the IgG is recovered from large scale purification of the plasma or fraction thereof. For example, 80%, or 81%, or 82%, or 83%, or 84% of IgG is recovered from the plasma or fraction thereof. In one example, 80% of the IgG is recovered from the plasma or fraction thereof. In one example, 81% of the IgG is recovered from the plasma or fraction thereof. In one example, 82% of the IgG is recovered from the plasma or fraction thereof. In one example, 83% of the IgG is recovered from the plasma or fraction thereof. In another example, 84% of the IgG is recovered from the plasma or fraction thereof.

In one example, at least 85% of the IgG is recovered from the plasma or fraction thereof. In another example, at least 85% of the IgG is recovered from the plasma or fraction thereof following the continuous chromatography method. For example, at least 85% of the IgG is recovered from the plasma or fraction thereof following the continuous chromatography method without further purification steps. For example, at least 85% of the IgG is recovered from the plasma or fraction thereof following the continuous chromatography method with further purification steps. For example, at least 85% of the IgG is recovered from the plasma or fraction thereof following an ion exchange chromatography step. In one example, at least 85% of the IgG is recovered from the plasma or fraction thereof following an anion exchange chromatography step. In one example, at least 85% of the IgG is recovered from the plasma or fraction thereof, wherein the IgG is derived from at least 500 kg of plasma or fractions thereof. For example, at least 85% of the IgG is recovered from large scale purification of the plasma or fraction thereof. For example, 85%, or 86%, or 87%, or 88%, or 89% of IgG is recovered from the plasma or fraction thereof. In one example, 85% of the IgG is recovered from the plasma or fraction thereof. In one example, 86% of the IgG is recovered from the plasma or fraction thereof. In one example, 87% of the IgG is recovered from the plasma or fraction thereof. In one example, 88% of the IgG is recovered from the plasma or fraction thereof. In another example, 89% of the IgG is recovered from the plasma or fraction thereof.

In one example, at least 90% of the IgG is recovered from the plasma or fraction thereof. In another example, at least 90% of the IgG is recovered from the plasma or fraction thereof following the continuous chromatography method. For example, at least 90% of the IgG is recovered from the plasma or fraction thereof following the continuous chromatography method without further purification steps. For example, at least 90% of the IgG is recovered from the plasma or fraction thereof following the continuous chromatography method with further purification steps. For example, at least 90% of the IgG is recovered from the plasma or fraction thereof following an ion exchange chromatography step. In one example, at least 90% of the IgG is recovered from the plasma or fraction thereof following an anion exchange chromatography step. In one example, at least 90% of the IgG is recovered from the plasma or fraction thereof, wherein the IgG is derived from at least 500 kg of plasma or fractions thereof. For example, at least 90% of the IgG is recovered from large scale purification of the plasma or fraction thereof. For example, 90%, or 91%, or 92%, or 93%, or 94% of IgG is recovered from the plasma or fraction thereof. In one example, 90% of the IgG is recovered from the plasma or fraction thereof. In one example, 91% of the IgG is recovered from the plasma or fraction thereof. In one example, 92% of the IgG is recovered from the plasma or fraction thereof. In one example, 93% of the IgG is recovered from the plasma or fraction thereof. In another example, 94% of the IgG is recovered from the plasma or fraction thereof.

In one example, at least 95% of the IgG is recovered from the plasma or fraction thereof. In another example, at least 95% of the IgG is recovered from the plasma or fraction thereof following the continuous chromatography method. For example, at least 95% of the IgG is recovered from the plasma or fraction thereof following the continuous chromatography method without further purification steps. For example, at least 95% of the IgG is recovered from the plasma or fraction thereof following the continuous chromatography method with further purification steps. For example, at least 95% of the IgG is recovered from the plasma or fraction thereof following an ion exchange chromatography step. In one example, at least 95% of the IgG is recovered from the plasma or fraction thereof following an anion exchange chromatography step. In one example, at least 95% of the IgG is recovered from the plasma or fraction thereof, wherein the IgG is derived from at least 500 kg of plasma or fractions thereof. For example, at least 95% of the IgG is recovered from large scale purification of the plasma or fraction thereof. For example, 95%, or 96%, or 97%, or 98%, or 99% of IgG is recovered from the plasma or fraction thereof. In one example, 95% of the IgG is recovered from the plasma or fraction thereof. In one example, 96% of the IgG is recovered from the plasma or fraction thereof. In one example, 97% of the IgG is recovered from the plasma or fraction thereof. In one example, 98% of the IgG is recovered from the plasma or fraction thereof. In another example, 99% of the IgG is recovered from the plasma or fraction thereof.

In one example, the eluted IgG has a purity of at least 95%. In another example, the eluted IgG has a purity of at least 95% following the continuous chromatography method. In one example, the eluted IgG has a purity of at least 95% following the continuous chromatography method without further purification steps. In one example, the eluted IgG has a purity of at least 95% following the continuous chromatography method with further purification steps. In one example, the eluted IgG having a purity of at least 95% is derived from at least 500 kg of plasma or fraction thereof. For example, the eluted IgG having a purity of at least 95% is recovered from large scale purification of the plasma or fraction thereof. For example, the eluted IgG has a purity of 95%, 96%, 97%, 98%, or 99%. In one example, the eluted IgG has a purity of 95%. In one example, the eluted IgG has a purity of 96%. In one example, the eluted IgG has a purity of 97%.

In one example, the eluted IgG has a purity of at least 98%. In another example, the eluted IgG has a purity of at least 98% following the continuous chromatography method. In one example, the eluted IgG has a purity of at least 98% following the continuous chromatography method without further purification steps. In one example, the eluted IgG has a purity of at least 98% following the continuous chromatography method with further purification steps. In one example, the eluted IgG having a purity of at least 98% is derived from at least 500 kg of plasma or fraction thereof. For example, the eluted IgG having a purity of at least 98% is recovered from large scale purification of the plasma or fraction thereof. For example, the eluted IgG has a purity of 98% or 99%.

In one example, the method is performed at large scale. For example, the method is performed on an industrial or a commercial scale. Methods of performing on an industrial or a commercial scale will be apparent to a skilled person and/or described herein. For example, the method performed on an industrial scale comprises large scale purification of IgG from the plasma or fraction thereof.

In one example, large scale purification of IgG is performed using at least 500 kg of the plasma or fraction thereof. For example, large scale purification of IgG is performed using between 500 kg to 1000 kg, or 1000 kg to 2500 kg, or 2500 kg to 5000 kg, or 5000 kg to 7500 kg, or 7500 kg, or 10000 kg, or 10000 kg to 12500 kg, or 12500 kg to 15000 kg of the plasma or fraction thereof. In one example, large scale purification of IgG is performed using at least 1000 kg, or 2500 kg, or 5000 kg, or 7500 kg, or 10000 kg, or 12500 kg, or 15000 kg of the plasma of fraction thereof. In one example, large scale purification of IgG is performed using at least 1000 kg of the plasma of fraction thereof.

In one example, large scale purification of IgG is performed using at least 2500 kg of the plasma of fraction thereof. In one example, large scale purification of IgG is performed using at least 5000 kg of the plasma of fraction thereof. In one example, large scale purification of IgG is performed using at least 7500 kg of the plasma of fraction thereof. In one example, large scale purification of IgG is performed using at least 10000 kg of the plasma of fraction thereof. In one example, large scale purification of IgG is performed using at least 12500 kg of the plasma of fraction thereof. In one example, large scale purification of IgG is performed using at least 15000 kg of the plasma of fraction thereof.

In one example, the method further comprises formulating the purified IgG into a pharmaceutical composition.

The present disclosure further provides a method for purifying IgG from plasma or fraction thereof using SMB chromatography, the method comprising:
a) equilibrating an affinity chromatography resin comprising a cross-linked poly(styrene-divinylbenzene) matrix and a ligand capable of specifically binding to a CH3 domain of human IgG with a 20 mM phosphate equilibration buffer having a pH of between 7 and 8;
b) binding the IgG from the plasma or fraction thereof to the resin;
c) washing the resin with a 20 mM phosphate wash buffer having a pH of between 7 and 8; and
d) eluting the bound IgG with a 20 mM acetate elution buffer having a pH of between 3 and 5;
   wherein steps a) to d) may be repeated on the affinity chromatography resin and wherein, the affinity chromatography resin is packed into a series of two or more fluidly-connected columns separated by fluid conduits comprising inlet and outlet valves, and optionally the method further comprises stripping the resin with a 20 mM phosphate wash buffer having a pH of between 2 and 3.

The present disclosure further provides a method for purifying IgG from plasma or fraction thereof using SMB chromatography, the method comprising:
a) equilibrating an affinity chromatography resin comprising a cross-linked poly(styrene-divinylbenzene) matrix and a ligand capable of specifically binding to a CH3 domain of human IgG with an equilibration buffer comprising 20 mM sodium dihydrogen phosphate buffer, 145 mM sodium chloride and having a pH of between 7 and 8;
b) binding the IgG from the plasma or fraction thereof to the resin;
c) washing the resin with a wash buffer comprising 20 mM sodium dihydrogen phosphate buffer, 145 mM sodium chloride and having a pH of between 7 and 8; and
d) eluting the bound IgG with a 20 mM acetate elution buffer having a pH of between 3 and 5;
   wherein steps a) to d) may be repeated on the affinity chromatography resin and wherein, the affinity chromatography resin is packed into a series of two or more fluidly-connected columns separated by fluid conduits comprising inlet and outlet valves, and optionally the method further comprises stripping the resin with a 20 mM phosphate wash buffer having a pH of between 2 and 3.

The present disclosure further provides a method for purifying IgG from plasma or fraction thereof using SMB chromatography, the method comprising:
a) equilibrating an affinity chromatography resin comprising a cross-linked poly(styrene-divinylbenzene) matrix and a ligand capable of specifically binding to a CH3 domain of human IgG with an equilibration buffer comprising 20 mM sodium dihydrogen phosphate buffer, 500 mM sodium chloride and having a pH of between 7 and 8;
b) binding the IgG from the plasma or fraction thereof to the resin;
c) washing the resin with a wash buffer comprising 20 mM sodium dihydrogen phosphate buffer, 500 mM sodium chloride and having a pH of between 7 and 8; and
d) eluting the bound IgG with a 20 mM acetate elution buffer having a pH of between 3 and 5;
   wherein steps a) to d) may be repeated on the affinity chromatography resin and wherein, the affinity chromatography resin is packed into a series of two or more fluidly-connected columns separated by fluid conduits comprising inlet and outlet valves, and optionally the method further comprises stripping the resin with a 20 mM phosphate wash buffer having a pH of between 2 and 3.

In one example, the method does not comprise stripping the resin.

The present disclosure further provides a method for purifying IgG from plasma or fraction thereof using SMB chromatography, the method comprising:
a) equilibrating an affinity chromatography resin comprising a cross-linked poly(styrene-divinylbenzene) matrix and a ligand capable of specifically binding to a CH3 domain of human IgG with a 20 mM phosphate equilibration buffer having a pH of between 7 and 8;
b) binding the IgG from the plasma or fraction thereof to the resin;
c) washing the resin with a 20 mM phosphate wash buffer having a pH of between 7 and 8; and
d) eluting the bound IgG with a 20 mM acetate elution buffer having a pH of between 3 and 5;
   wherein steps a) to d) may be repeated on the affinity chromatography resin and wherein, the affinity chromatography resin is packed into a series of two or more fluidly-connected columns separated by fluid conduits comprising inlet and outlet valves, and wherein the method does not comprise stripping the resin.

The present disclosure further provides a method for purifying IgG from plasma or fraction thereof using SMB chromatography, the method comprising:
a) equilibrating an affinity chromatography resin comprising a cross-linked poly(styrene-divinylbenzene) matrix and a ligand capable of specifically binding to a CH3 domain of human IgG with a 20 mM phosphate equilibration buffer having a pH of between 7 and 8;
b) binding the IgG from the plasma or fraction thereof to the resin;
c) washing the resin with a 20 mM phosphate wash buffer having a pH of between 7 and 8; and
d) eluting the bound IgG with a 20 mM acetate or phosphate elution buffer having a pH of between 3 and 5;
   wherein steps a) to d) may be repeated on the affinity chromatography resin and wherein, the affinity chromatography resin is packed into a series of two or more fluidly-connected columns separated by fluid conduits comprising inlet and outlet valves, and optionally wherein the method does not comprise stripping the resin.

The present disclosure further provides a method for purifying IgG from plasma or fraction thereof using SMB chromatography, the method comprising:
a) equilibrating an affinity chromatography resin comprising a cross-linked poly(styrene-divinylbenzene) matrix and a ligand capable of specifically binding to a CH3 domain of human IgG with an equilibration buffer comprising 20 mM sodium dihydrogen phosphate buffer, 145 mM sodium chloride and having a pH of between 7 and 8;
b) binding the IgG from the plasma or fraction thereof to the resin;
c) washing the resin with a wash buffer comprising 20 mM sodium dihydrogen phosphate buffer, 145 mM sodium chloride and having a pH of between 7 and 8; and
d) eluting the bound IgG with a 20 mM acetate elution buffer having a pH of between 3 and 5;
   wherein steps a) to d) may be repeated on the affinity chromatography resin and wherein, the affinity chromatography resin is packed into a series of two or more fluidly-connected columns separated by fluid conduits comprising inlet and outlet valves, and wherein the method does not comprise stripping the resin.

The present disclosure further provides a method for purifying IgG from plasma or fraction thereof using SMB chromatography, the method comprising:
a) equilibrating an affinity chromatography resin comprising a cross-linked poly(styrene-divinylbenzene) matrix and a ligand capable of specifically binding to a CH3 domain of human IgG with an equilibration buffer comprising 20 mM sodium dihydrogen phosphate buffer, 500 mM sodium chloride and having a pH of between 7 and 8;
b) binding the IgG from the plasma or fraction thereof to the resin;
c) washing the resin with a wash buffer comprising 20 mM sodium dihydrogen phosphate buffer, 500 mM sodium chloride and having a pH of between 7 and 8; and
d) eluting the bound IgG with a 20 mM acetate elution buffer having a pH of between 3 and 5;
   wherein steps a) to d) may be repeated on the affinity chromatography resin and wherein, the affinity chromatography resin is packed into a series of two or more fluidly-connected columns separated by fluid conduits comprising inlet and outlet valves, and wherein the method does not comprise stripping the resin.

In one example, the method is repeated on the resin for at least 50 cycles. For example, the method is repeated on the resin for at least 50 cycles per batch of plasma or fraction thereof. In an example, the method is repeated on the resin for between 50 to 80 cycles, 60 to 80 cycles, for 70 to 80 cycles per batch of plasma or fraction thereof. For example, the method is repeated on the resin for at least 60, or 65, or 70, or 75, or 80 cycles per batch of plasma or fraction thereof.

In one example, the method is repeated on the resin for 50 cycles per batch of plasma or fraction thereof.

In one example, the method is repeated on the resin for 60 cycles per batch of plasma or fraction thereof.

In one example, the method is repeated on the resin for 70 cycles per batch of plasma or fraction thereof.

In one example, the method is repeated on the resin for 80 cycles per batch of plasma or fraction thereof.

In one example, the method is repeated on the resin with multiple batches of plasma or fractions thereof. For example, the method is repeated on the resin with at least two batches of plasma or fractions thereof. In one example, the method is repeated on the resin with 2, or 3, or 4, or 5, or 6, or 7, or 8, or 9, or 10 batches of plasma or fractions thereof. In one example, the method is repeated on the resin with between 4 to batches of plasma or fractions thereof.

In one example, the method is repeated on the resin for up to a total of 800 cycles. For example, the resin is reused for up to a total number of 800 cycles. In one example, the method is repeated on the resin for up to a total of 100, or 200, or 300, or 400, or 500, or 600, or 700 cycles. For example, the method is repeated on the resin for up to a total of 100 cycles. For example, the method is repeated on the resin for up to a total of 200 cycles. For example, the method is repeated on the resin for up to a total of 300 cycles. For example, the method is repeated on the resin for up to a total of 400 cycles. For example, the method is repeated on the resin for up to a total of 500 cycles. For example, the method is repeated on the resin for up to a total of 600 cycles. For example, the method is repeated on the resin for up to a total of 700 cycles.

In one example, the method is repeated on the resin for between 100 to 200 cycles, or 200 to 300 cycles, or 200 to 500 cycles, or 500 to 800 cycles.

In one example, the method is repeated on the resin for 200 cycles.

In one example, the method is repeated on the resin for 300 cycles.

In one example, the method is repeated on the resin for 400 cycles.

In one example, the method is repeated on the resin for 500 cycles.

In one example, the method is repeated on the resin for 600 cycles.

In one example, the method is repeated on the resin for 700 cycles.

In one example, the method is repeated on the resin for 800 cycles.

In one example, the method is repeated on the resin for between 200 and 500 cycles. For example, the resin is reused for up to a total number of 200 to 500 cycles. In one example, the resin is reused up to a total of up to 500 cycles with up to 10 batches of plasma or fractions thereof.

In one example, the sanitization step is performed on the resin after each individual cycle. In another example, the sanitisation step is performed on the resin after multiple cycles. For example, the sanitisation step is performed on the resin after at least 50 cycles. In one example, the sanitisation step is performed on the resin after at least 100 cycles. In another example, the sanitisation step is performed on the resin after at least 150 cycles. In a further example, the sanitisation step is performed on the resin after at least 200 cycles. In one example, the sanitisation step is performed on the resin after each batch of plasma or fractions thereof. For example, the sanitisation step is performed on the resin between each batch of plasma or fractions thereof, i.e., before the loading of each batch of plasma or fractions thereof on to the resin.

Suitable sanitisation methods will be known to the skilled person and/or described herein.

In one example, the method reduces the DBC of the resin. For example, reuse of the resin reduces the DBC of the resin. In one example, the DBC of the resin is reduced by at up to 80%. For example, the DBC of the resin is reduced by at up to 75%, or 70%, or 65%, or 60%, or 55%, or 40%, or 45%, or 40%, or 35%, or 30%, or 25%, or 20%, or 15%, or 10%, or 5%.

In one example, the resin is reused until the DBC of the resin is reduced by up to 80%.

In one example, the method reduces the DBC of the resin by 80%. For example, the resin is reused until the DBC of the resin is reduced by 80%.

In one example, the method reduces the DBC of the resin by 70%. For example, the resin is reused until the DBC of the resin is reduced by 70%.

In one example, the method reduces the DBC of the resin by 60%. For example, the resin is reused until the DBC of the resin is reduced by 60%.

In one example, the method reduces the DBC of the resin by 50%. For example, the resin is reused until the DBC of the resin is reduced by 50%.

In one example, the method reduces the DBC of the resin by 40%. For example, the resin is reused until the DBC of the resin is reduced by 40%.

In one example, the method reduces the DBC of the resin by 30%. For example, the resin is reused until the DBC of the resin is reduced by 30%.

In one example, the method reduces the DBC of the resin by 20%. For example, the resin is reused until the DBC of the resin is reduced by 20%.

In one example, the method reduces the DBC of the resin by 10%. For example, the resin is reused until the DBC of the resin is reduced by 10%.

The present disclosure also provides a pharmaceutical composition comprising IgG purified or produced by a method described herein. For example, the pharmaceutical composition comprises IgG purified or produced by a method described herein and a pharmaceutically acceptable carrier.

In one example, the pharmaceutical composition comprises at least 1% (w/v) purified IgG. For example, the pharmaceutical composition comprises 1% (w/v) purified IgG. In another example, the pharmaceutical composition comprises 5% (w/v) purified IgG. In one example, the pharmaceutical composition comprises between 10 and 30% (w/v) purified IgG. For example, the pharmaceutical composition comprises 10% (w/v) purified IgG. In one example, the pharmaceutical composition comprises 16.5% (w/v) purified IgG. In another example, the pharmaceutical composition comprises 20% (w/v) purified IgG. In one example, the pharmaceutical composition comprises 25% (w/v) purified IgG. In another example, the pharmaceutical composition comprises 30% (w/v) purified IgG.

In one example, the IgG content in the pharmaceutical composition is at least 95% (w/w) of the total amount of protein in the composition. For example, the IgG content in the pharmaceutical composition is 95% (w/w) of the total amount of protein in the composition. In another example, the IgG content in the pharmaceutical composition is 96% (w/w) of the total amount of protein in the composition. In a further example, the IgG content in the pharmaceutical composition is 97% (w/w) of the total amount of protein in the composition. In one example, the IgG content in the pharmaceutical composition is 98% (w/w) of the total amount of protein in the composition. In a further example, the IgG content in the pharmaceutical composition is 99% (w/w) of the total amount of protein in the composition.

In one example, the pharmaceutical composition comprises 100 mg/mL of total human plasma protein. In one example, the pharmaceutical composition comprises 20 g/100 mL of total human plasma protein.

In one example, the pharmaceutical composition comprises a purity of at least 95% immunoglobulin G (IgG). For example, the pharmaceutical composition comprises a purity of at least 96% immunoglobulin G (IgG). In another example, the pharmaceutical composition comprises a purity of at least 97% immunoglobulin G (IgG). In another example, the pharmaceutical composition comprises a purity of at least 98% immunoglobulin G (IgG). In another example, the pharmaceutical composition comprises a purity of at least 99% immunoglobulin G (IgG).

In one example, the pharmaceutical composition comprises an IgG1 subclass distribution of at least 60%. For example, the pharmaceutical composition comprises an IgG1 subclass distribution of at least 65%.

In one example, the pharmaceutical composition comprises an IgG2 subclass distribution of less than 30%. For example, the pharmaceutical composition comprises an IgG2 subclass distribution of less than 28%.

In one example, the pharmaceutical composition comprises an IgG3 subclass distribution of less than 5%. For example, the pharmaceutical composition comprises an IgG3 subclass distribution of less than 4%.

In one example, the pharmaceutical composition comprises an IgG4 subclass distribution of less than 5%. For example, the pharmaceutical composition comprises an IgG4 subclass distribution of less than 3%.

In one example, the pharmaceutical composition comprises an IgG subclass distribution that is similar to that of normal human plasma, for example 69% IgG₁, 26% IgG₂, 3% IgG₃ and 2% IgG₄.

In one example, the pharmaceutical composition comprises a nominal osmolality of between about 300 mOsm/kg and 400 mOsm/kg. In one example, the pharmaceutical composition comprises a nominal osmolality of 380 mOsm/kg. For example, the pharmaceutical composition comprises a nominal osmolality of between about 300 mOsm/kg and 350 mOsm/kg. In one example, the pharmaceutical composition comprises a nominal osmolality of 320 mOsm/kg.

In one example, the pharmaceutical composition comprises a pH of between 4 and 5.5. For example, the pharmaceutical composition comprises a pH of between 4.5 and 5.0. In one example, the pharmaceutical composition comprises a pH of between 4.6 and 5.0. For example, the pharmaceutical composition comprises a pH of 4.6. In one example, the pharmaceutical composition comprises a pH of 4.7. In another example, the pharmaceutical composition comprises a pH of 4.8. In a further example, the pharmaceutical composition comprises a pH of 4.9. In one example, the pharmaceutical composition comprises a pH of 5.0.

In one example, the pharmaceutical composition further comprises 200 mmol/L to 300 mmol/L of L-proline. For example, the pharmaceutical composition further comprises 225 mmol/L to 275 mmol/L of L-proline. In one example, the pharmaceutical composition further comprises 240 mmol/L to 260 mmol/L of L-proline. For example, the pharmaceutical composition further comprises 250 mmol/L of L-proline.

In one example, the pharmaceutical composition comprises a sodium content of ≤1 mmol/L.

In one example, the pharmaceutical composition comprises an IgA content of ≤0.05 mg/mL. For example, the pharmaceutical composition comprises an IgA content of ≤0.04 mg/mL, or ≤0.03 mg/mL. In one example, the pharmaceutical composition comprises an IgA content of ≤0.025 mg/mL. In one example, the pharmaceutical composition comprises an IgA content of ≤0.01 mg/mL. For example, the pharmaceutical composition comprises an IgA content of ≤0.009 mg/mL.

In one example, the pharmaceutical composition comprises an IgA content of ≤0.1 mg/g IgG. In one example, the pharmaceutical composition comprises an IgA content of ≤0.09 mg/g IgG.

In one example, the pharmaceutical composition comprises an IgM content of K 10 mg/L. For example, an IgM content of ≤10 mg/L, ≤9 mg/L, ≤8 mg/L, ≤7 mg/L, ≤6 mg/L, ≤5 mg/L, ≤4 mg/L, ≤3 mg/L, ≤2 mg/L. In one example, the pharmaceutical composition comprises an IgM content of ≤2 mg/L. In one example, the pharmaceutical composition comprises an IgM content of ≤1 mg/L. In one example, the pharmaceutical composition comprises an IgM content of ≤0.5 mg/L. For example, the pharmaceutical composition comprises an IgM content of <0.17 mg/L.

In one example, the pharmaceutical composition comprises an IgM content of ≤2 μg/g IgG. In one example, the pharmaceutical composition comprises an IgM content of ≤1.9 μg/g IgG.

In one example, the pharmaceutical composition comprises an albumin content of ≤0.50 mg/mL. For example, the pharmaceutical composition comprises an albumin content of ≤0.40 mg/mL. In one example, the pharmaceutical composition comprises an albumin content of ≤0.30 mg/mL. In one example, the pharmaceutical composition comprises an albumin content of ≤0.20 mg/mL. In one example, the pharmaceutical composition comprises an albumin content of ≤0.10 mg/mL. For example, the pharmaceutical composition comprises an albumin content of ≤0.09 mg/mL. In one example, the pharmaceutical composition comprises an albumin content of ≤0.08 mg/mL. In one example, the pharmaceutical composition comprises an albumin content of ≤0.07 mg/mL.

In one example, the pharmaceutical composition comprises an albumin content of ≤1 mg/g IgG. In one example, the pharmaceutical composition comprises an albumin content of ≤0.80 mg/g IgG.

In one example, the pharmaceutical composition comprises a Prekallikrein activator (PKA) level of ≤35 IU/mL. In one example, the pharmaceutical composition comprises a Prekallikrein activator (PKA) level of ≤30 IU/mL. In one example, the pharmaceutical composition comprises a Prekallikrein activator (PKA) level of ≤50 IU/mL. In one example, the pharmaceutical composition comprises a Prekallikrein activator (PKA) level of ≤20 IU/mL. For example, the pharmaceutical composition comprises a Prekallikrein activator (PKA) level of ≤15 IU/mL. In one example, the pharmaceutical composition comprises a Prekallikrein activator (PKA) level of ≤10 IU/mL.

The present disclosure also provides the pharmaceutical composition described herein for use in treating, preventing and/or delaying progression of a condition in a subject. For example, the present disclosure provides a pharmaceutical composition described herein for use in treating a condition in a subject. In another example, the present disclosure provides a pharmaceutical composition described herein for use in preventing a condition in a subject. In a further example, the present disclosure provides a pharmaceutical composition described herein for use in delaying progression of a condition in a subject.

In some examples, the pharmaceutical composition is present in a vial, a prefilled syringe or an autoinjector device.

The present disclosure also provides a prefilled syringe comprising the pharmaceutical composition described herein.

The present disclosure also provides an autoinjector device comprising the pharmaceutical composition described herein.

In one example, the composition of the disclosure is administered subcutaneously to the subject in need thereof. In another example, the composition of the disclosure is administered intravenously to the subject in need thereof.

In one example, the composition of the disclosure is self-administered.

In one example, the composition of the disclosure is self-administered subcutaneously.

In one example, the composition of the disclosure is provided in a pre-filled syringe.

In one example, the composition of the disclosure is self-administered subcutaneously, with a pre-filled syringe.

The present disclosure further provides use of IgG purified or produced by a method described herein in the manufacture of a medicament for treating, preventing and/or delaying progression of a condition in a subject. For example, the present disclosure provides use of the IgG purified or produced by a method described herein in the manufacture of a medicament for treating a condition in a subject. In another example, the present disclosure provides use of the IgG purified or produced by a method described herein in the manufacture of a medicament for preventing a condition in a subject. In a further example, the present disclosure provides use of the IgG purified or produced by a method described herein in the manufacture of a medicament for delaying progression of a condition in a subject.

The present disclosure also provides a method of treating, preventing and/or delaying progression of a condition in a subject, the method comprising administering the pharmaceutical composition of the present disclosure to the subject. For example, the present disclosure provides a method of treating a condition in a subject. In another example, the present disclosure provides a method of preventing a condition in a subject. In a further example, the present disclosure provides a method of delaying progression of a condition in a subject.

The present disclosure also provides a kit for use in treating or preventing or delaying progression of a condition in a subject, the kit comprising:
(a) at least one pharmaceutical composition described herein;
(b) instructions for using the kit in treating or preventing or delaying the condition in the subject; and
(c) optionally, at least one further therapeutically active compound or drug.

In one example, the condition is an immunodeficiency, autoimmune disease or acute infection. For example, the condition is allogenic bone marrow transplant, chronic lymphocytic leukaemia, idiopathic thrombocytopenic purpura (ITP), pediatric HIV, primary immunodeficiencies, Kawasaki disease, chronic inflammatory demyelinating polyneuropathy (CIDP), kidney transplant with a high antibody recipient or with an ABO incompatible donor, chronic fatigue syndrome, Clostridium difficile colitis, dermatomyositis and polymyositis, Graves' ophthalmopathy, Guillain-Barre syndrome, muscular dystrophy, inclusion body myositis, Lambert-Eaton syndrome, Lupus erythematosus, multifocal motor neuropathy, multiple sclerosis (MS), myasthenia gravis, neonatal alloimmune thrombocytopenia, Parvovirus B19 infection, pemphigus, post-transfusion purpura, renal transplant rejection, spontaneous Abortion Miscarriage, stiff person syndrome, opsoclonus Myoclonus, severe sepsis and septic shock in critically ill adults, toxic epidermal necrolysis, chronic lymphocytic leukemia, multiple myeloma, X-linked agammaglobulinemia, hypogammaglobulinemia, primary immune deficiency, RRMS, Alzheimer's disease, and Parkinson's disease.

In one example, the condition is selected from a group consisting of primary immunodeficiency disease (PI), chronic inflammatory demyelinating polyneuropathy (CIDP), and chronic immune thrombocytopenic purpura (ITP).

In one example the condition is primary immunodeficiency disease (PI).

In one example, the condition is chronic inflammatory demyelinating polyneuropathy (CIDP).

In one example, the condition is chronic immune thrombocytopenic purpura (ITP).

In one example of any method described herein, the subject is a mammal, for example a primate such as a human.

### BRIEF DESCRIPTION OF THE DRAWINGS

FIG. 1 is (A) SDS-PAGE gel image of clarified cryo-poor plasma FcXP POROS® eluate (FcXP) under reducing (left) and non-reducing (right) conditions and (B) Table of protein impurities identified in the eluate from the SDS-PAGE gel run.

FIG. 2 is a gel image of a 2D-DIGE of proteins in the eluate.

FIG. 3 is a graphical representation showing IgG subclass distribution of cryo-rich plasma (CRP) and cryo-poor plasma (CPP) prior to use in method described herein, CRP and CPP eluate (i.e. eluates from the FcXP resin).

FIG. 4 is a graphical representation showing of static binding capacity of FcXP POROS® resin over successive runs at 6 cm (LTS1) and 20 cm (LTS2) bed heights.

FIG. 5 is a graphical representation showing of pro-coagulant activity of (A) plasma and (B) cryo-poor plasma (CPP) as a result of temperature over time, or filtration as determined by NaPTT assay. Coagulation time was set at >150s.

FIG. 6 is a graphical representation showing proteolytic activity of thrombin (S-2238), general serine proteases (S-2288), kallikrein (S-2302), plasmin (S-2251) and FXa (S-2765) as a result of temperature over time in (A) plasma and (B) cryo-poor plasma (CPP).

FIG. 7 is a graphical representation showing viral inactivation of CRP using N-Octyl-β-D-Glucopyranoside.

FIG. 8 is a graphical representation showing (A) temperature dependent volume-normalized ratio of cryoprecipitate in samples thawed at different temperatures; and (B) hold time study schematic to evaluate optimal thawing and hold time temperature, respectively.

FIG. 9 is a series of graphical representations showing back pressure during the SMB process (A) with a strip phase and (B) without a strip phase.

FIG. 10 is a series of graphical representations showing (A) a reduction in proteolytic activity in eluate (i.e. eluate from the FcXP resin) with increasing wash buffer conductivity and (B) a reduction in proteolytic activity in eluate (i.e. eluates from the FcXP resin) from normal and cryo-poor plasma (CPP) as a result of increasing wash buffer conductivity from 145 mM sodium chloride to 500 mM sodium chloride.

FIG. 11 is a series of graphical representations showing (A) IgG yield, (B) product purity using Lapchip assay and (C) Albumin, IgA and IgM levels in normal plasma and cryo-poor plasma (CPP) in eluates (i.e. eluates from the FcXP resin) using a wash buffer comprising 145 mM or 500 mM sodium chloride.

### KEY TO SEQUENCE LISTING

SEQ ID NO: 1 is an amino acid sequence of VHH fragment
SEQ ID NO: 2 is an amino acid sequence of CDR1 of the VHH fragment
SEQ ID NO: 3 is an amino acid sequence of CDR2 of the VHH fragment
SEQ ID NO: 4 is an amino acid sequence of CDR3 of the VHH fragment

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

Furthermore, as used herein the singular forms of “a”, “and” and “the” include plural references unless the context clearly dictates otherwise.

### Selected Definitions

The term “purify” or “purifying” or “purification” shall be taken to mean the removal, whether completely or partially, of at least one impurity present in the plasma or fraction thereof, which thereby improves the level of purity of IgG in solution.

The term “impurity” or “impurities” shall be taken to include one or more components in the plasma or fraction thereof other than IgG. For example, impurities may include albumin (α-globulins and/or β-globulins), plasma lipids, plasma proteins, proteases (e.g. serine proteases, kallikrein, plasmin and FXa), serine protease inhibitors (e.g. C1 inhibitor, alpha-1-antitrypsin and anti-thrombin), IgA and IgM, factor VIII, fibrinogen, von Willebrand factor, activated clotting factors (e.g. FXa, FIXa, FVIIa and thrombin), factor XIII, contact system factors (e.g. FXIa, FXIIa and plasma kallikrein), PKA, factor IX, prothrombin complex, C1 esterase inhibitor, protein C, anti-thrombin III, a RhD immunoglobulin and platelet membrane microparticles.

The term “immunoglobulin G (IgG)”, also known as “gamma globulin” or “immune globulin”, shall be taken to mean antibody of isotype G. There are several subclasses of IgG, for example, IgG1, IgG2, IgG3 and IgG4.

The term “plasma” shall refer to the straw-coloured/pale yellow component of blood obtained from one or more blood donor(s). Methods of obtaining plasma from a donor will be apparent to a skilled person and/or described herein. For example, plasma is obtained by removing red blood cells from donated blood. For example, plasma is obtained by plasmapheresis.

The term “plasma fraction” or “fraction thereof” shall refer to plasma which has been fractionated to isolate one or more desirable protein components from the plasma.

For example, plasma may be fractionated to isolate cryo-precipitates (proteins that precipitate out of solution when a unit of fresh frozen plasma is slowly thawed in the cold) and cryosupernatant (also known as cryo-poor plasma). For example, plasma may be fractionated by ethanol precipitation to produce IgG-containing Oncley fractions, Cohn fractions, ammonium sulphate precipitates, or Precipitates A (KN A), B (KN B), and the Precipitate of Supernatant B (KN B+1) from plasma as described in U.S. Pat. No. 3,301,842. Plasma fractions include II+III precipitate produced according to Cohn methods such as Method 6, Cohn et. al. J. Am; Chem. Soc., 68 (3), 459-475 (1946), Method 9, Oncley et al. J. Am; Chem. Soc., 71, 541-550 (1946), or the I+II+III precipitate, Method 10, Cohn et. al. J. Am; Chem. Soc., 72, 465-474 (1950); as well as the method of Deutsch et. al. J. Biol. Chem. 164, 109-118 (1946) or the Precipitate-A, B and the Precipitate of Supernatant B of Nitschmann and Kistler Vox Sang. 7, 414-424 (1962); Helv. Chim. Acta 37, 866-873 (1954). For example, the plasma may be fractionated by octanoic acid fractionation as described in European application 893450. Typically, Cohn Fractions, and Kistler/Nitschmann Precipitate's A (KN A), B (KN B) and the Precipitate of Supernatant B (KN B+1) exist as a suspended paste. Other purification techniques including chromatography may be used.

The term “cryo-precipitate” or “cryo-precipitates” refers to proteins in plasma that precipitate out of solution when a unit of fresh frozen plasma is slowly thawed in the cold. Cryo-precipitates include factor VIII, fibrinogen, von Willebrand factor, factor XIII and platelet membrane microparticles.

The term “cryo-poor plasma” shall be taken to mean plasma removed of cryo-precipitates.

The term “cryo-rich plasma” shall be taken to mean plasma comprising components typically found in cryo-precipitates.

The term “clarified” or “clarifying” shall be taken to mean a process of passing a plasma or fraction thereof through a suitable filter (e.g. depth filter and/or 1.2 and 0.45/0.22 μm membrane filter) to remove one or more impurities prior to use in a method described herein.

The term “dissociation constant” shall refer to the pKa of a buffer. pKa=−log₁₀ (Ka), wherein Ka is the acid dissociation constant of the buffering agent of the buffer. For example, a wash buffer of 20 mM sodium dihydrogen phosphate, 40 mM sodium chloride at a pH of 7.4 comprises sodium dihydrogen phosphate as the buffering agent. Phosphoric acid has three dissociation constants (pKa1: 2.16, pKa2: 7.21, pKa3: 12.32).

The term “affinity chromatography resin” shall be taken to mean a resin comprising an affinity chromatography ligand (e.g. camelid-derived single domain [VHH] antibody fragment) attached to a matrix such as, e.g., those described herein. Exemplary affinity chromatography resins used in a method described herein include POROS® CaptureSelect® FcXP affinity resin (Thermo Fisher) and CaptureSelect® FcXP agarose affinity resin (Thermo Fisher). Further exemplary affinity chromatography resins include a resin having an amino acid sequence encoded by SEQ ID NO: 1 or variants thereof that specifically bind to the CH3 domain of human IgG. Exemplary affinity chromatography resins are also described in U.S. Ser. No. 10/259,886.

The term “specifically binds”, “specifically binding” or “binds specifically” shall be taken to mean that a protein of the disclosure reacts or associates more frequently, more rapidly, with greater duration and/or with greater affinity with a particular antigen or cell expressing same than it does with alternative antigens or cells. For example, a ligand capable of specifically binding to a CH3 domain of human IgG with materially greater affinity (e.g., 1.5 fold or 2 fold or 5 fold or 10 fold or 20 fold or 40 fold or 60 fold or 80 fold to 100 fold or 150 fold or 200 fold) than it does to other antigens. Generally, but not necessarily, reference to binding means specific binding, and each term shall be understood to provide explicit support for the other term.

The term “ligand” shall be taken to mean a molecule immobilised to a matrix of the affinity chromatography resin which specifically binds to the CH3 domain of human IgG. For example, the ligand is a camelid-derived single domain [VHH] antibody fragment.

The term “enriched preparation” shall be taken to include an eluate, solution or pharmaceutical composition described herein. The enriched preparation of the present disclosure comprises IgG at greater purity compared to IgG in the plasma or fraction thereof.

The term “camelid-derived single domain [VHH] antibody fragment” shall be taken to mean a VHH domain of a camelidae antibody. The camelidae antibody is an antibody produced from camels and llamas and has no CH1 domain normally present in human immunoglobulins and only one VHH domain. Exemplary affinity chromatography resins comprising the camelid-derived [VHH] antibody fragment include CaptureSelect® antibody affinity chromatography resins (Thermo Fisher). For example, CaptureSelect® FcXL affinity resin, POROS® CaptureSelect® FcXP affinity resin, CaptureSelect IgG-CH1 affinity resin, and CaptureSelect FcXP agarose affinity resin. Further exemplary affinity chromatography resins include IgSelect® affinity resin (Cytiva), HiTrap® IgSelect® affinity resin (Cytiva), Pierce® Protein G agarose affinity resin (Thermo Fisher), and Protein G sepharose 4 fast flow affinity resin (Cytiva).

The term “matrix” shall be taken to mean a support to which the ligand is immobilised. Exemplary matrices are cross-linked poly(styrene-divinylbenzene) matrix and agarose-based matrix.

The term “dynamic binding capacity” or “DBC” of a chromatography resin shall be taken to refer to the maximum amount of IgG that the resin will bind under operating conditions before significant breakthrough of unbound IgG occurs.

The term “per mL of resin” shall be taken to refer to per mL of wet packed volume of resin.

The term “bed height” shall be taken to mean the height at which the affinity chromatography resin is packed into a column. It will be apparent to the skilled person that reference to “total bed height” refers to the bed height of all columns in the continuous chromatography set-up.

The term “non-loading phase” shall be taken to mean a phase other than the loading phase of the continuous chromatography method. For example, a non-loading phase can refer to an equilibration phase, a wash phase, an elution phase, a strip phase and/or a re-equilibration phase.

The term “cycle” shall be taken to mean one round of equilibrating, IgG loading, binding, elution, stripping, sanitising, and/or regenerating performed on the resin.

The term “purity” shall refer to the portion of IgG relative to the total protein content of purified IgG, IgG enriched preparation and pharmaceutical composition expressed as a percentage.

The term “industrial or commercial scale” or “large scale” or “manufacturing scale” shall refer to the amount of product that would be produced in a batch that was designed for clinical testing, formulation, sale and/or distribution to the public. For example, industrial scale refers to large scale purification of IgG from the plasma or fraction thereof to produce the plasma protein product.

The term “plasma protein product” shall refer to a preparation, composition and/or protein product comprising a plasma protein (e.g. IgG or impurity such as albumin) derived from the purification of the plasma or fraction thereof. Typically, the plasma protein is the predominant protein in the plasma protein product.

The term “pharmaceutical composition” shall be taken to mean a formulation of IgG with compounds generally accepted in the art for the delivery of IgG to mammals. Exemplary compounds include all pharmaceutically acceptable carriers, diluents or excipients thereof.

The term “treat” or “treatment” or “treating” shall be taken to mean administering a therapeutically effective amount of IgG such that one or more symptoms or characteristics of the condition is reduced in the subject or that the subject is no longer clinically diagnosed with the condition.

The term “preventing”, “prevent” or “prevention” includes providing prophylaxis with respect to occurrence or recurrence of a specified condition in a subject. A subject may be predisposed to or at risk of developing a condition but has not yet been diagnosed with the condition.

As used herein, the phrase “delaying progression of” includes reducing or slowing down the progression of a condition in a subject and/or at least one symptom of the condition.

The term “condition” shall be taken to mean a state of being or health status of a subject in need of treatment with IgG. Exemplary conditions include but are not limited to primary immunodeficiency disease (PI), chronic inflammatory demyelinating polyneuropathy (CIDP), and chronic immune thrombocytopenic purpura (ITP).

The term “subject” shall be taken to mean any animal including humans, for example a mammal. Exemplary subjects include but are not limited to humans and non-human primates. For example, the subject is a human.

### Continuous Affinity Chromatography

The present disclosure provides a method of purifying IgG from plasma or a fraction thereof using continuous affinity chromatography.

The term “continuous affinity chromatography” shall be taken to mean a chromatographic method comprising one or more column(s) packed with identical affinity resins, wherein each column comprises one or more zones. A zone is a column, or a region of a column, comprising the resin where one or more chromatography steps can be performed. For example, a zone is selected from a group consisting of an equilibration zone, a binding zone, a wash zone, an elution zone, a stripping zone, or a combination thereof. In one example, a zone is selected from a group consisting of an equilibration zone, a binding zone, a wash zone, an elution zone, or a combination thereof.

Continuous affinity chromatography comprising more than one column involves the columns being connected in an arrangement that allows the columns to be operated in series and/or in parallel. In principle, IgG may be loaded on a first and/or subsequent columns while other columns (or other zones of a column) are going through an equilibration, wash, elution, and/or regeneration simultaneously. Examples of continuous affinity chromatography will be apparent to the skilled person and/or described herein.

Examples of columns which may be used to perform the continuous chromatography method will be apparent to the skilled person and/or described herein. For example, the continuous chromatography method may be performed using Tricorn 5/100 (Cytiva). In another example, the continuous chromatography method may be performed using BioSMB PD System (Sartorius).

### Simulated Moving Bed (SMB) Chromatography

In one example, the continuous affinity chromatography is simulated moving bed (SMB) chromatography. The term “simulated moving bed chromatography” or “SMB chromatography” refers to a chromatography method first described in U.S. Pat. No. 2,985,589. Examples of SMB chromatography setup and/or apparatus will be apparent to the skilled person and/or described herein. The concept of simulated moving bed involves the use of multiple smaller columns (rather than one large column) containing a solid absorbent (e.g. affinity resin) and performing one or more continuous chromatography steps (i.e. equilibration, binding, washing, eluting or stripping) simultaneously on different columns in a continuous loop.

An example of a SMB chromatography set up has columns arranged into four sections with one or more columns per section. Two inlet streams (feed and eluent) and two outlet streams (extract and raffinate) are directed in alternating order to and from the column ring. The inlet and outlet positions are switched at regular time intervals in the direction of the liquid flow, thus simulating counter-current movement of columns. A feed (containing adsorbable components (extract)) is loaded onto one or more columns of the SMB chromatography setup, and the extract binds to the resin within the columns. Meanwhile, less adsorbed components (raffinate) in the feed pass through the column. The raffinate may be loaded onto one or more subsequent column(s) or removed from the SMB chromatography system as waste. An eluent is loaded onto the column to collect the extract. For example, an eluate may be collected from a first column while more feed is loaded onto one or more subsequent column(s).

Suitable wash and elution buffers having the characteristics of the present disclosure will be apparent to the skilled person and/or described herein. In one example, the wash buffer comprises 20 mM sodium dihydrogen phosphate, 145 mM sodium chloride and is at a pH of 7.4. In one example, the wash buffer comprises 20 mM sodium dihydrogen phosphate, 500 mM sodium chloride and is at a pH of 7.4.

The resin in SMB chromatography may undergo multiple cycles (e.g. 50 cycles) of resin equilibration, IgG loading, binding, elution, stripping, sanitising, and/or regeneration per batch of plasma or fraction thereof used. Multiple batch runs (e.g. 4 to batches) may be performed using SMB chromatography. The total life time of the resin in SMB chromatography can be in the range of 200 to 500 cycles (if not more) before the resin is unusable. Resin regeneration is generally performed to allow multiple uses of the resin.

### Periodic Counter-Current Chromatography (PCC)

In one example, the continuous affinity chromatography is periodic counter-current chromatography (PCC). Examples of PCC setup and/or apparatus will be apparent to the skilled person and/or described herein. The concept of PCC involves the use of multiple columns containing a solid absorbent (e.g. affinity resin) and performing the chromatography steps in parallel in a quasi-continuous manner. The buffers used in binding, washing, and/or elution steps flow counter-current to the affinity resin.

An example of PCC setup involves the use of two columns. In a first step, a sample is loaded onto a first column above the DBC of the resin so that unbound product (e.g. IgG) breaks through the first column and is captured by the second column. In a second step, the first column is washed, eluted, cleaned and/or re-equilibrated independently of the second column being loaded with a further sample. In a third step, an additional sample is loaded onto the second column above the DBC of the resin so that unbound product breaks through the second column and is captured by the first column. In a fourth step, the second column is washed, eluted, cleaned and/or re-equilibrated independently of the first column being loaded with a further sample. The process steps are continuously cycled between the two columns.

Another example of PCC setup involves the use of multiple columns. For example, a variation of the above PCC setup can involve use of multiple columns to capture unbound product which simulates use of a large column.

### Continuous Counter-Current Tangential Chromatography (CCTC)

In one example, the continuous affinity chromatography is continuous counter-current tangential chromatography (CCTC). Examples of CCTC setup and/or apparatus will be apparent to the skilled person and/or described herein. The concept of CCTC involves using the affinity resin in a slurry form where the slurry is continuously directed through a number of static mixers and hollow fiber membranes which separate the fluid phase from the resin. CCTC is ordinarily performed at low pressures.

An example of a CCTC process involves binding, first wash, second wash, elution, stripping and/or equilibration steps. Another example of a CCTC process involves binding, first wash, second wash, elution and/or equilibration steps. For example, the CCTC process does not involve a stripping step. Sample (e.g. plasma or fraction thereof) and the affinity resin is passed through static mixers and hollow fiber membranes in a binding step. Impurities are removed in the flow through of the hollow fiber membranes in the washing step, while resin bound product (i.e. IgG) is retained by the membrane. The hollow fibres retain the resin and allow the product to flow through in the elution step. The resins are stripped and/or equilibrated and process repeated.

### Continuous Counter-Current Spiral Chromatography (CCSC)

In one example, the continuous affinity chromatography is continuous counter-current spiral chromatography (CCSC). Examples of CCSC setup and/or apparatus will be apparent to the skilled person and/or described herein. The concept of CCSC involves the use of a compact rotating coil separation column mounted onto a centrifuge rotary frame. There are two separation column designs currently available: the spiral disk assembly and the spiral tube support assembly.

An exemplary CCSC process involves a coiled separation column revolving around a central axis of the centrifuge while it synchronously rotates about its own axis (at e.g., 1,000 to 1,200 rpm). A mobile phase can be passed through the centrifuge rotor without rotary seals, and a large amount of a stationary phase is retained while the two phases are mixed along the length of the column to produce a highly efficient solute separation.

### Affinity Chromatography Resin

The present disclosure provides a method of purifying immunoglobulin G (IgG) from the plasma or fraction thereof using an affinity chromatography resin. The affinity resin of the present disclosure comprises a ligand capable of specifically binding to a CH3 domain of human IgG.

Suitable affinity chromatography resins will be apparent to the skilled person and/or described herein. In one example, the resin comprises a ligand of camelid-derived single domain [VHH] antibody fragments. The skilled person will be aware that ligands based on camelid-derived single domain [VHH] antibody fragments are capable of specifically binding to all subclasses of IgG (IgG1, IgG2, IgG3, IgG4). Exemplary resins are the CaptureSelect® FcXP affinity chromatography resins (Thermo Fisher), CaptureSelect® FcXL affinity resin (Thermo Fisher), CaptureSelect® IgG-CH1 affinity resin (Thermo Fisher), and CaptureSelect® FcXP agarose affinity resin (Thermo Fisher). Further exemplary affinity chromatography resins include IgSelect® affinity resin (Cytiva), HiTrap® IgSelect® affinity resin (Cytiva), Pierce® Protein G agarose affinity resin (Thermo Fisher), and Protein G sepharose 4 fast flow affinity resin (Cytiva).

In one example, the affinity chromatography resin comprises a camelid-derived single domain [VHH] antibody fragment and a cross-linked poly(styrene-divinylbenzene) matrix. For example, the affinity chromatography resin is POROS® CaptureSelect® FcXP affinity resin (Thermo Fisher). The cross-linked poly(styrene-divinylbenzene) matrix allows the resin to withstand pressures of up to 100 bar.

In one example, the affinity chromatography resin comprises a camelid-derived single domain [VHH] antibody fragment and an agarose-based matrix. For example, the affinity chromatography resin is CaptureSelect FcXP agarose affinity resin (Thermo Fisher).

In one example, the continuous affinity chromatography process is performed at a pressure in the range of about 2 to about 5 bar. For example, the continuous affinity chromatography process is performed at a pressure in the range of about 3 to about 4 bar. In one example, the continuous affinity chromatography process is performed at a pressure in the range of about 3.25 to about 3.5 bar.

### Buffers

The present disclosure provides a continuous affinity chromatography method using buffers which enable efficient IgG binding to, and collection from, the resin. Generally, plasma or fraction thereof are at a neutral pH (pH of about 7.4). The resin is equilibrated with an equilibration buffer and/or washed with a wash buffer having a buffering range which covers the neutral pH. Suitable wash buffers comprise buffering agents having a dissociation constant (pKa) between 6.8 and 8.5 at 25° C.

An exemplary buffering agent of the equilibration and/or wash buffer is sodium dihydrogen phosphate, where the phosphoric acid component of sodium dihydrogen phosphate has three dissociation constants (pKa: 2.16, 7.21 and 12.32). Phosphoric acid has a dissociation constant at about the pH of an elution and/or stripping buffer used in the continuous affinity chromatography method. However, phosphoric acid does not have a dissociation constant between the pH of the equilibration and/or wash buffer (higher pH) and the elution and/or stripping buffer (lower pH) used in the continuous affinity chromatography method. This enables a fast switch between wash and elution steps, and stripping and equilibration steps, giving more defined peaks and shorter chromatography phases. An advantage of using such equilibration and/or wash buffers is that smaller buffer volumes can be used, thereby increasing the efficiency of the continuous affinity chromatography method.

Other suitable buffering agents of the equilibration and/or wash buffer include imidazole (pKa: 7.0), Tris (pKa: 8.30), glycylglycine (pKa: 8.40), MOPS (pKa: 7.2), PIPES (pKa: 6.8), TES (pKa: 7.40), Bicine (pKa: 8.35), HEPES (pKa: 7.55), EPPS (pKa: 8.00), HEPPSO (pKa: 7.85), MOBS (pKa: 7.60), POPSO (pKa: 7.78), TAPSO (pKa: 7.61), Tricine (pKa: 8.05), TEA (pKa: 7.76).

### Analysis of IgG Composition

Methods of determining yield, purity and IgG subclass distribution will be apparent to the skilled person and/or described herein.

In one example, purity is determined by SDS-PAGE and MALDI-TOF-MS peptide fingerprint analysis. Briefly, purified IgG, an IgG-enriched preparation or IgG-containing pharmaceutical composition described herein is loaded onto a suitable SDS-PAGE gel (e.g. 8-16% TRIS-glycine), along with a protein size marker and a positive control for IgG (e.g. Privigen) under reduced and non-reduced conditions. Proteins are separated based on size and protein bands of interest are isolated, processed and analysed by MALDI-TOF-MS.

In another example, impurities in the IgG-enriched preparation or IgG-containing pharmaceutical composition described herein are measured in an Enzyme-Linked Immunosorbent Assay (ELISA) using impurity (e.g. IgA) specific antibodies. For example, the ELISA is performed using commercially available methods. In one example, purity, yield and/or subclass distribution of IgG is determined by nephelometry. In one example, purity of IgG is determined by nephelometry. In one example, yield of IgG is determined by nephelometry. In one example, subclass distribution of IgG is determined by nephelometry. For example, the light scattering patterns of purified IgG, an IgG-enriched preparation or IgG-containing pharmaceutical composition described herein is measured by nephelometry and compared to light scattering profiles of compositions with known IgG subclass distributions.

### Stability of Plasma and Fractions Thereof

The stability of the plasma or fraction thereof for loading onto an affinity resin described herein can be determined by assessing the pro-coagulant activity, proteolytic activity and particle size of the plasma or fraction thereof. Methods for assessing pro-coagulant activity, proteolytic activity and particle size will be apparent to a skilled person and/or described herein. Briefly, the plasma or fraction thereof is freeze/thawed in one or more cycles, stored at between 2° C. and 32° C. (e.g. 2° C., 10° C., 18° C., 21° C., 28° C. or 32° C.) for 24 or up to 48 hrs and analysed using one or more of the methods described below. In one example, the plasma or fraction thereof is thawed in one or more cycles at a temperature of 32° C., stored for 24 or up to 48 hours and analysed using one or more of the methods described below. In another example, the plasma or fraction thereof is thawed in one or more cycles at a temperature of 32° C., stored for 24 or up to 48 hours and analysed using one or more of the methods described below and then cooled and stored at a temperature of 21° C. In one example, the plasma or fraction thereof is thawed at a temperature of 32° C. and at a temperature of 21° C. before the continuous affinity chromatography.

In one example, the pro-coagulant activity in the plasma or fraction thereof can be determined using an in vitro coagulation assay, e.g., activated partial thromboplastin time (NaPTT) assay. The NaPTT assay measures the rate at which one or more coagulation factors (e.g., fibrinogen, prothrombin, proaccelerin, anti-hemophilic factor, Stuart-Prower factor, plasma thromboplastin antecedent and Hegeman factor) are activated or form in plasma, or a fraction thereof, when coagulation activators (e.g. silica, kaolin, ellagic acid) are added to the assay.

In one example, proteolytic activity in the plasma or fraction thereof can be assessed by measuring the activity of thrombin, general serine proteases, kallikrein, plasmin and FXa e.g. using commercially available kits, such as thrombin activity assay kit (S-2238), general serine protease assay kit (S-2288), kallikrein activity assay kit (S-2302), plasmin activity assay kit (S-2251) and FXa activity kit (S-2765).

In one example, the size of any particles in the plasma or fraction thereof is assessed by microflow imaging (MFI) and polydispersity index is calculated. Calculation of the polydispersity index will be apparent to the skilled person.

### Additional Purification Steps

Additional purification steps may be performed before or after the continuous chromatography step. In one example, additional purification steps may be performed before the continuous chromatography step. In one example, additional purification steps may be performed after the continuous chromatography step.

In one example, the method further comprises one or more steps selected from a group consisting of ethanol precipitation, octanoic acid fractionation, ion exchange chromatography, viral inactivation, viral filtration and ultrafiltration/diafiltration. Additional purification steps will be apparent to the skilled person and/or described herein.

In one example, the method further comprises ethanol precipitation. For example, cold ethanol may be used to isolate and enrich IgG by removing albumin and α- and β-globulins from the plasma or fractions thereof. For example, as described in WO2011/149472.

In one example, the method further comprises immunoaffinity chromatography. For example, the method further comprises isoagglutinin affinity chromatography using Eshmuno anti-A and anti-B resin. For example, isoagglutinin affinity chromatography may be used to remove isoagglutinins A and B.

In one example, the method further comprises octanoic acid fractionation. Octanoic acid may be used to remove of plasma lipids and plasma proteins (other than IgG). For example, as described in WO2011/131787.

In one example, the method further comprises ion exchange chromatography. In one example, the ion exchange chromatography is anion exchange chromatography. For example, anion exchange chromatography may be used to remove IgA, remaining IgM and other plasma components (other than IgG).

The anion exchanger can be a resin-based anion exchanger, an anion exchange membrane adsorber, or any other format of anion exchanger with a positively charged substrate for capturing negatively charged particles. In one example, the anion exchanger is an anion exchange membrane adsorber. In another example, the anion exchanger is a resin-based anion exchanger. In a further example, the anion exchanger is a monolithic anion exchanger.

In one example, the method further comprises anion exchange chromatography using a resin-based anion exchanger. For example, the anion exchange chromatography resin is a strong anion exchanger. In one example, the strong anion exchange resin comprises a matrix consisting of a poly(styrene-divinylbenzene) matrix. In one example, the strong anion exchanger comprises a quaternized polyethyleneimine functional group. Suitable resin-based anion exchanges will be apparent to the skilled person and include, for example, POROS™ HQ 50.

In one example, the anion exchange chromatography step is performed in flow through mode. In another example, the anion exchange chromatography step is performed in bind-and-elute mode.

In one example, the anion exchange chromatography step comprises a buffer selected from the group consisting of sodium citrate, 2-(N-morpholino)ethanesulfonic acid (MES) buffer, sodium dihydrogen phosphate, Bis-Tris, phosphate, L-histidine and combinations thereof. In one example, the anion exchange chromatography step comprises a buffer comprising MES buffer. In another example, the anion exchange chromatography step comprises phosphate buffer.

In one example, the method further comprises viral inactivation. For example, viral inactivation may be effected by adjusting the solution to low pH. Low pH may be a pH of between 2 to 4. In one example, low pH viral inactivation is performed in the presence of caprylate. In another example, viral inactivation may be effected by contacting the plasma or fraction thereof, or an IgG-enriched preparation or IgG-containing pharmaceutical composition with n-Octyl-β-D-Glucopyranoside (OG), thereby forming an OG-IgG mixture. In a further example, low pH viral inactivation is performed in the presence of N,N-Dimethylmyristylamine N-oxide (TDAO).

In a further example, viral inactivation may be effected by exposing the plasma or fraction thereof, or an IgG-enriched preparation or IgG-containing pharmaceutical composition to a solvent-detergent inactivation step. Suitable solvent-detergent treatments would be apparent to the skilled person and include, for example environmentally friendly detergents. Exemplary environmentally friendly detergents suitable for use in the present disclosure and in particular for use in inactivating lipid enveloped viruses include N,N-Dimethylmyristylamine N-oxide (TDAO), polysorbate 80 (PS80), polyoxyethylene (10) isooctylcyclohexyl ether (TRITON® X-100-reduced), and a non-ionic surfactant prepared from glucose and alcohol (e.g., Simulsol™ formulations). In one example, the detergent is N,N-Dimethylmyristylamine N-oxide (TDAO). In one example, the detergent is polysorbate 80. In another example, the detergent is polyoxyethylene (10) isooctylcyclohexyl ether (TRITON® X-100-reduced). In a further example, the detergent is a non-ionic surfactant prepared from glucose and alcohol.

In one example, the OG concentration in the OG-IgG mixture is in the range of 25 mM to 80 mM. For example, the OG concentration in the OG-IgG mixture is in the range of 25 mM to 50 mM, or 50 mM to 80 mM, or 30 mM to 60 mM. For example, OG concentration in the OG-IgG mixture is 25 mM, or 30 mM, or 35 mM, or 40 mM, or 45 mM, or 50 mM, or 55 mM, or 60 mM, or 65 mM, or 70 mM, or 75 mM, or 80 mM.

In one example, the OG concentration in the OG-IgG mixture is 30 mM.

In one example, the plasma or fraction thereof, or the IgG-enriched preparation or IgG-containing pharmaceutical composition is contacted with OG for up to 15 minutes. For example, the plasma or fraction thereof, or the IgG-enriched preparation or IgG-containing pharmaceutical composition is contacted with OG for up to 0.5 minutes, or 1 minute, or 1.5 minutes, or 2 minutes, or 2.5 minutes, or 3 minutes, or 3.5 minutes, or 4 minutes, or 4.5 minutes, or 5 minutes, or 5.5 minutes, or 6 minutes, or 6.5 minutes, or 7 minutes, or 7.5 minutes, or 8 minutes, or 8.5 minutes, or 9 minutes, or 9.5 minutes, or 10 minutes, or 10.5 minutes, or 11 minutes, or 11.5 minutes, or 12 minutes, or 12.5 minutes, or 13 minutes, or 13.5 minutes, or 14 minutes, or 14.5 minutes, or 15 minutes.

In one example, the plasma or fraction thereof, or the IgG-enriched preparation or IgG-containing pharmaceutical composition is contacted with OG at a temperature in the range of from 2° C. to 28° C. For example, the plasma or fraction thereof, or the IgG-enriched preparation or IgG-containing pharmaceutical composition is contacted with OG at a temperature in the range of from 2° C. to 8° C., or 2° C. to 28° C., or 2° C. to 25° C., or 2° C. to 20° C., or 2° C. to 18° C., or 2° C. to 15° C., or 2° C. to 10° C. For example, the plasma or fraction thereof, or the IgG-enriched preparation or IgG-containing pharmaceutical composition is contacted with OG at a temperature of 2° C., or 3° C., or 4° C., or 5° C., or 6° C., or 7° C., or 8° C., or 9° C., or 10° C., or 11° C., or 12° C., or 13° C., or 14° C., 15° C., or 16° C., or 17° C., or 18° C., or 19° C., or 20° C., or 21° C., or 22° C., or 23° C., or 24° C., or 25° C., or 26° C., or 27° C., or 28° C.

In one example, the plasma or fraction thereof, or the IgG-enriched preparation or IgG-containing pharmaceutical composition is contacted with OG at a temperature of 2° C.

In one example, the plasma or fraction thereof, or the IgG-enriched preparation or IgG-containing pharmaceutical composition is contacted with OG at a temperature of 8° C.

In one example, the plasma or fraction thereof, or the IgG-enriched preparation or IgG-containing pharmaceutical composition is contacted with OG at a temperature of 10° C.

In one example, the plasma or fraction thereof, or the IgG-enriched preparation or IgG-containing pharmaceutical composition is contacted with OG at a temperature of 18° C.

In one example, the plasma or fraction thereof, or the IgG-enriched preparation or IgG-containing pharmaceutical composition is contacted with OG at a temperature of 28° C.

In one example, the method further comprises viral filtration. For example, viral filtration membranes of pore sizes from 15-20 nm may be used to remove microbes and viruses from a solution or eluate or pharmaceutical composition. Exemplary nanofilters include Planova S20N (Asahi), Virosart HC (Sartorius) and Planova 20N (Asahi).

In one example, the method further comprises ultrafiltration/diafiltration. An exemplary ultrafiltration/diafiltration membrane is Pellicon 2 Cassettes (Millipore) or Polyethersulfone or Hydrosart cassettes (Sartorius).

### Pharmaceutical Compositions

Purified IgG of the disclosure (syn. active ingredients) are useful for formulations into a pharmaceutical composition for parenteral, such as intravenous administration or subcutaneous administration, for therapeutic and prophylactic treatment.

The compositions for administration will commonly comprise a solution of the purified IgG of the disclosure dissolved in a pharmaceutically acceptable carrier, such as an aqueous carrier. A variety of aqueous carriers can be used, e.g., buffered saline and the like. The compositions may contain pharmaceutically acceptable carriers as required to approximate physiological conditions such as pH adjusting and buffering agents, toxicity adjusting agents and the like, for example, sodium acetate, sodium chloride, potassium chloride, calcium chloride, sodium lactate and the like.

The concentration of the purified IgG of the present disclosure in these formulations can vary widely, and will be selected primarily based on fluid volumes, viscosities, body weight and the like in accordance with the particular mode of administration selected and the patient's needs. The vehicles may contain minor amounts of additives that enhance isotonicity and chemical stability, e.g., buffers and preservatives. For example, the pharmaceutical composition comprises proline as a stabilising agent.

Suitable pharmaceutical compositions in accordance with the disclosure will generally include an amount of the purified IgG of the present disclosure admixed with an acceptable pharmaceutical carrier, such as a sterile aqueous solution, to give a range of final concentrations, depending on the intended use. The techniques of preparation are generally known in the art as exemplified by Remington's Pharmaceutical Sciences, 16th Ed. Mack Publishing Company, 1980.

For example, the IgG concentration of the pharmaceutical composition is 1 to 5% w/v, 5 to 15% w/v, or 8 to 12% w/v. For example, the IgG concentration of the pharmaceutical composition is 1%, 2%, 3%, 4%, 5%, or 6%, or 7%, or 8%, or 9%, or 10%, or 11%, or 12%, or 13%, or 14%, or 15% w/v. For intravenous use, 1% w/v (i.e. 10 g IgG/L) may be used. For intravenous use, 10% w/v (i.e. 100 g IgG/L) may be used.

For subcutaneous administration, a higher concentration may be used. For example, 15 to 35% w/v, or 20 to 30% w/v. In one example, the IgG concentration of the pharmaceutical composition is 16%, or 17%, or 18%, or 19%, or 20%, or 21%, or 22%, or 23%, or 24%, or 25%, or 26% w/v.

### Method of Use

As discussed herein, the present disclosure provides a method of treating, preventing and/or delaying progression of a condition in a subject, comprising administering IgG or a pharmaceutical formulation to the subject.

In one example, the condition is selected from a group consisting of primary immunodeficiency disease (PI), chronic inflammatory demyelinating polyneuropathy (CIDP), and chronic immune thrombocytopenic purpura (ITP).

### EXAMPLES

### Example 1: Affinity Resin

Affinity chromatography resins POROS® CaptureSelect® FcXP affinity resin (Thermo Fisher) and CaptureSelect® FcXP agarose affinity resin (Thermo Fisher) were used to evaluate suitable resins for capturing IgG from plasma or a fraction thereof. Both affinity chromatography resins contain a ligand capable of binding to a CH3 domain of human IgG, specifically camelid-derived single domain [VHH] antibody fragment, and a matrix of either cross-linked poly(styrene-divinylbenzene) or agarose. The affinity chromatography resins were packed in Tricorn columns from Cytiva (diameter 5 mm) and chromatography performed on an Äkta avant 25 system (Cytiva).

Cryo-rich plasma (CRP) and cryo-poor plasma (CPP) (prepared by CSL Behring) were warmed to 37° C. and filtered through a 0.22 μm bottle-top filter. The affinity chromatography resins were evaluated using chromatography running conditions and buffers provided by Thermo Fischer described in Table 1 below.

TABLE 1
Affinity Chromatography buffers used to evaluate FcXP resins
for the purification of IgG from human CCP and CPP.

| Chromatography phase | Buffer |
|---|---|
| Equilibration | 10 mM citric acid buffer, 150 mM sodium |
| | chloride, pH 7.4 |
| Column Wash | 10 mM citric acid buffer, 150 mM sodium |
| | chloride, pH 7.4 |
| Elution | 20 mM sodium acetate buffer, pH 4.0 |
| Column stripping | 100 mM citric acid buffer, pH 2.0 |
| Eluate neutralization | 1M Tris base stock, diluted to 20 mM in |
| | final sample |

Purification of IgG from CRP and CPP using POROS® CaptureSelect® FcXP affinity resin resulted in higher yields of IgG recovered and slightly higher purity of eluent compared to use of CaptureSelect® FcXP agarose affinity resin. Based on these initial results, the suitability of POROS® CaptureSelect® FcXP affinity resin for use in continuous affinity chromatography was further evaluated.

### Example 2: Buffer Composition

A shortcoming of the wash buffer of Table 1 is that the buffering agent citric acid has three dissociation constants (pKa1: 3.13, pKa2: 4.76, pKa3: 6.4) and does not suitably buffer at a pH of 7.4 required for equilibration. Furthermore, the pH shift between washing and elution, and strip and equilibration using buffers comprising citric acid (as provided in Table 1) results in inefficient pH changes and elongated phases during chromatography.

To determine suitable buffer compositions to enable fast adjustment of pH during continuous affinity chromatography, experiments performed in Example 1 was repeated using POROS® CaptureSelect® FcXP affinity resin with chromatography buffers of Table 2 used instead of Table 1.

TABLE 2
Affinity Chromatography buffers used
to evaluate POROS ® CaptureSelect ® FcXP
affinity resin for the of IgG from human
CRP and CPP.

| Chromatography phase | Buffer |
|---|---|
| Equilibration | 20 mM sodium dihydrogen phosphate buffer, |
| | 145 mM sodium chloride, pH 7.4 |
| Column Wash | 20 mM sodium dihydrogen phosphate buffer, |
| | 145 mM sodium chloride, pH 7.4 |
| Elution | 20 mM sodium acetate buffer, pH 4.0 |
| Column stripping | 20 mM sodium dihydrogen phosphate buffer, |
| | pH 2.5 |

Sodium dihydrogen phosphate present in the wash buffer has three dissociation constants (pKa1: 2.16, pKa2: 7.21 and pKa3: 12.32) which covered the pH of 7.4 required for equilibration of the resin. Increasing the buffer concentration of sodium dihydrogen phosphate to 20 mM was found to excluded effects of insufficient buffering during the experiments. However, lower concentrations of sodium dihydrogen phosphate may be used in the wash buffer.

The results showed that use of sodium dihydrogen phosphate enables fast switching between low pH during elution and stripping and high pH during equilibration and wash giving more defined peaks and shorter phases with the continuous affinity chromatography runs.

No difference in the binding behaviour of IgG was observed between pH 7.4 and 7.8, allowing the CRP and CPP with pH residing with this range to be applied to the resin without pH adjustment.

### Example 3: Bed Height and Flow Rates

To determine conditions that enable a reduction of loading and non-loading phases, bed heights and flow rates used in the chromatography process was evaluated. Experiments described in Example 2 were repeated at different bed heights of the resin and with different flow rates of the plasma or fraction thereof and elution buffer applied to the resin.

Bed heights of 20 cm showed significantly higher efficiency of the purification of IgG from the plasma or fraction thereof compared to lower bed heights, enabling short non-loading phases. The reduction of the flow rate to 150 cm/hr (5 minutes contact time) during elution showed no significant effect compared to elution at 2400 cm/h. Surprisingly, it was found that the loading flow rate at which the plasma or fraction thereof is applied to the resin was increased to 2400 cm/hr (0.5 minutes contact time) without significant reduction in IgG binding.

Optimised conditions for purifying IgG from the plasma or fraction thereof determined by the extensive experimentation is summarised in Table 3 below. The combination of buffer compositions, resin, bed height, and flow rates selected resulted in reduced volume of waste, flow through and product during each of the non-loading phases (i.e. equilibration, wash, elution, strip and re-equilibration).

TABLE 3
Optimized conditions for purifying IgG from human CRP
and CPP using POROS ® CaptureSelect ® FcXP
affinity resin. Column bed height in a 5 mm diameter
column was approximately 20 cm. The contact time was
0.5 min, resulting in a flow rate of up to 2400 cm/hr.

| Chromatography | | | |
|---|---|---|---|
| phase | Input | Volumes | Outlet |
| Equilibration | Equilibration buffer | 0.5 CV | Waste |
| Load | Clarified cryo | 4.3 CV | Flow through |
| | poor plasma | | |
| | (c[IgG] = 7 mg/ml) | | |
| Wash | Equilibration buffer | 1.5 CV | Flow through |
| Wash 2 | Elution buffer | 0.5 CV | Flow through |
| Elution | Elution buffer | 1.8 CV | Product |
| Strip | Strip buffer | 1.8 CV | Waste |
| Re equilibration | Equilibration buffer | 2.5 CV | Waste |

Using columns of 20 cm bed height, the combined volume of the non-loading phases was 8.6 CV while the load volume of the clarified CPP was 4.3 CV. The non-load/load CV ratio was 2, enabling a SMB setup with just three columns. A similar result was observed using a column bed height of approximately 6 cm. The combined volume of the non-loading phases in experiments conducted in columns of approximately 6 cm bed height was 7 CV while the load volume of the clarified CPP was 3.8. The non-load/load CV ratio was 1.8, enabling a SMB setup of just three columns rather than 4 columns. The results demonstrate that irrespective of bed height, reducing the volume of the non-loading phases allows for the use of fewer columns in the purification method.

Furthermore, IgG was eluted in 1.8 CV giving a concentration factor of 2.4. The cycle time (excluding pump ramp-up and pump washes) was approximately 6.5 minutes.

Further densification is possible by overlapping elution and strip phases as for wash and elution phases. An increase of the buffer concentration or pH during re-equilibration would further reduce the buffer needed enabling a permanent non-load/load CV ratio below 2, even where the amount of the plasma or fraction thereof loaded is reduced due to resin aging.

### Example 4: Eluate Profile

### Purity

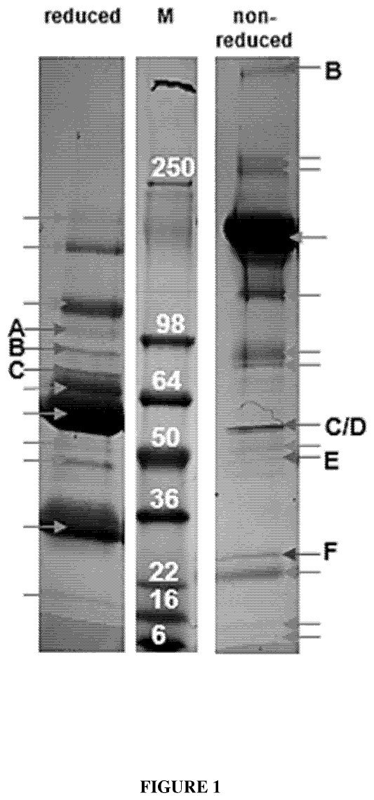

To determine the purity qualitatively of the eluate of Examples 2 and 3, SDS-PAGE gel electrophoresis was performed. Samples (8 μg and 16 μg) of IgG purified from clarified CPP using FcXP resin according to Examples 2 and 3 (FcXP) were run on 8-16% TRIS-glycine SDS-PAGE gel under reduced and non-reduced conditions (FIG. 1). Protein marker (M) See Blue Plus 2 Marker (Invitrogen) was also included. The SDS-PAGE gels were Coomassie stained. The purity of IgG from clarified CPP purified using POROS® CaptureSelect® FcXP affinity resin as determined by SDS-PAGE was 98.7%.

To determine the impurity profile of the eluate of Examples 2 and 3, MALDI-TOF-MS peptide mass fingerprint were performed. Visible bands (marked by arrows in FIG. 1) were isolated and used for MALDI-TOF-MS peptide mass fingerprinting to determine the identity of the proteins at each band. Impurities were identified by the MALDI-TOF-MS peptide mass fingerprinting (marked by arrows A-F in FIG. 1) and summarised in Table 4. The remaining bands (arrows without designated letters in FIG. 1) were identified as IgG. The most abundant impurities in FcXP samples were IgM, albumin and apolipoprotein A-1 (arrows B, C and F in FIG. 1). The other three components of the complement system were minor impurities (arrows A, D and E in FIG. 1 and bands A, D and E in Table 4).

TABLE 4
Impurities present in the eluate eluted from the POROS ®
CaptureSelect FcXP affinity resin identified by MALDI-TOF-MS

| Bands | Impurity |
|---|---|
| A | C1r subcomponent |
| B | IgM |
| C | HSA |
| D | C1r subcomponent subunit B |
| E | C1q subcomponent subunit C |
| F | Apolipoprotein A-1 |

SE-HPLC showed that an average monomer and dimer content of IgG in the eluate was 98.1% with 0.9% polymer and 1.0% fragment content.

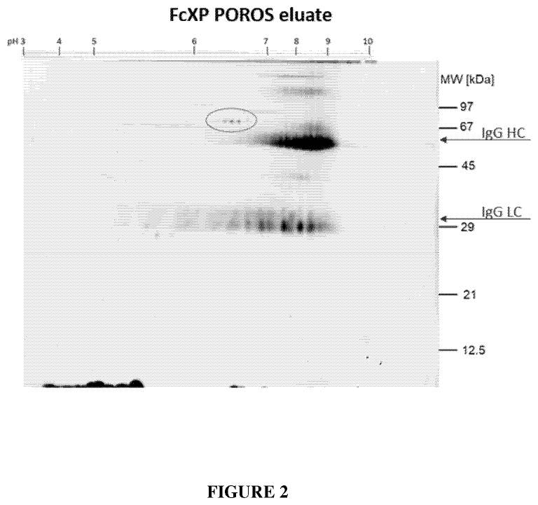

To determine the isoelectric point (pI) of impurities in the eluate compared to IgG, 2D Difference Gel Electrophoresis (2D DIGE) was performed. Samples of purified IgG from clarified CPP (FcXP POROS® eluate) were loaded with Sci5 and Sci3 dye respectively and loaded onto a 2D-SDS-PAGE. Separation of the samples by isoelectric point was performed between pH 3 and 10, followed by separation by size in the presence of SDS under reducing conditions (FIG. 2). IgG was identified to have isoelectric points between pH 7 and 9 in FcXP POROS® eluate samples (FIG. 2). IgM and albumin were identified in the eluate to have a pH of approximately 6.5 (circle in FIG. 2) which is lower than the isoelectric points of IgG.

The results suggest that removal of IgM and albumin from the eluate is possible using an ion exchange polishing step without loss of significant amounts of IgG.

### IgG Subclass Distribution

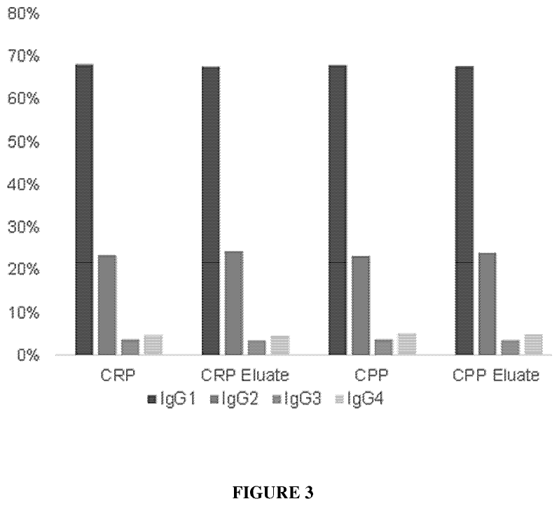

To determine whether the affinity resin used in purification of IgG affects the subclass distribution of IgG in the eluate of Examples 2 and 3, immunonephelometry was performed. IgG subclass distribution was determined for CRP, CPP, purified IgG from CRP or CPP using POROS® CaptureSelect® FcXP (FIG. 3). The subclass distribution was calculated by the relative part of IgG class in relation to sum of all classes (IgGx/(IgG1+IgG2+IgG3+IgG4).

### Yield

Yield was determined using immunonephelometry. At least 95% of IgG was recovered in the eluate from plasma of Examples 2 and 3, which was as high as 96%.

### Example 5: Purification Cycling

To determine the influence of purification cycling on POROS® CaptureSelect® FcXP affinity resin, multiple purification cycles were performed successively, and binding capacity of the resin was determined. At various points during the multiple purification cycles, the breakthrough behaviour of pure IgG was determined and used to calculate the remaining binding capacity of the resin. Loss of binding capacity may be attributed to aging of the resin.

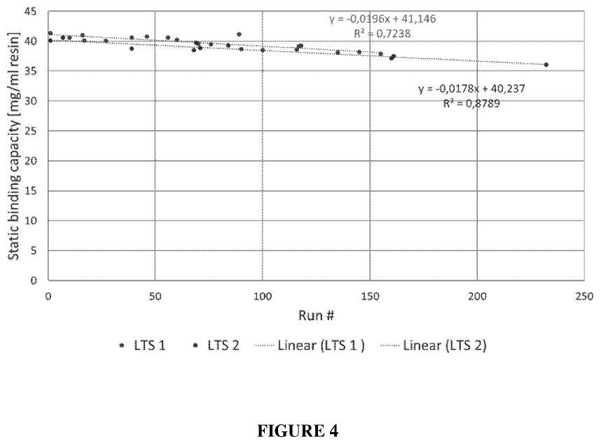

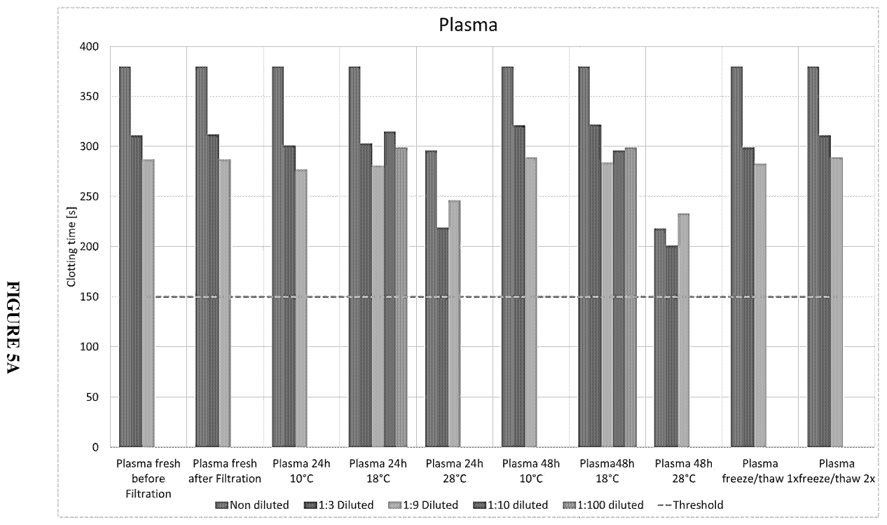

CRP or CPP was applied to the resin (bed height of either 6 cm or 20 cm) to allow the plasma to contact the resin for 0.5 minutes for each phase and binding capacity of the resin was determined over time. FIG. 4 shows that there was no difference in the resin aging between SMB chromatography performed with a resin of bed height of 6 cm and 20 cm. A decrease in binding capacity followed a linear trend with an average slope of <5% per 100 runs, demonstrating only small resin aging even after 100 runs. The results demonstrate that POROS® CaptureSelect FcXP® affinity resin is suitable for use in a SMB chromatography setup under conditions described in Examples 2 and 3.

### Example 6: Stability of Plasma and Fraction Thereof

The stability of the CRP and CPP for use in a method described herein was assessed. CRP and CPP were freeze thawed up to twice and/or filtered using a 0.22 μm filter. The processed CRP and CPP were stored at 10° C., 18° C., or 28° C. for 24 or 48 hrs. IgG content, as well as IgG subclass distribution, of the samples was determined by immunonephelometry. The results of Tables 5 and 6 show that the IgG content and IgG subclass distribution of CRP and CPP were unaffected by filtration, temperature, time and freeze/thawing.

TABLE 5
IgG content of CRP with and without filtration, freeze/thaw
and storage at set times and temperatures.

| | Absolute | | | | |
|---|---|---|---|---|---|
| | values | Subclass distribution | | | |
| | IgG | IgG1 | IgG2 | IgG3 | IgG4 |
| Sample | (g/L) | % | % | % | % |
| Plasma fresh before filtration | 5.48 | 54 | 32 | 3 | 11 |
| Plasma fresh after filtration | 5.54 | 54 | 32 | 3 | 10 |
| Plasma 24 hr 10° C. | 5.47 | 54 | 32 | 3 | 10 |
| Plasma 24 hr 18° C. | 5.38 | 54 | 32 | 3 | 11 |
| Plasma 24 hr 28° C. | 5.5 | 54 | 32 | 3 | 11 |
| Plasma 48 hr 10° C. | 5.35 | 54 | 32 | 3 | 11 |
| Plasma 48 hr 18° C. | 5.47 | 54 | 32 | 3 | 11 |
| Plasma 48 hr 28° C. | 5.59 | 54 | 32 | 3 | 10 |
| Plasma freeze/thaw 1x | 5.53 | 53 | 33 | 3 | 11 |
| Plasma freeze/thaw 2x | 5.58 | 54 | 33 | 3 | 10 |

TABLE 6
IgG content of CPP with and without filtration, freeze/thaw
and storage at set times and temperatures.

| | Absolute | | | | |
|---|---|---|---|---|---|
| | values | Subclass distribution | | | |
| | IgG | IgG1 | IgG2 | IgG3 | IgG4 |
| Sample | (g/L) | % | % | % | % |
| CPP fresh before filtration | 6.42 | 65 | 25 | 4 | 6 |
| CPP fresh after filtration | 6.5 | 65 | 25 | 4 | 6 |
| CPP 24 hr 10° C. | 6.51 | 65 | 26 | 3 | 6 |
| CPP 24 hr 18° C. | 6.64 | 65 | 26 | 3 | 6 |
| CPP 24 hr 28° C. | 6.58 | 65 | 25 | 4 | 6 |
| CPP 48 hr 10° C. | 6.46 | 65 | 25 | 4 | 6 |
| CPP 48 hr 18° C. | 6.46 | 66 | 25 | 4 | 6 |
| CPP 48 hr 28° C. | 6.52 | 66 | 25 | 3 | 6 |
| CPP freeze/thaw 1x | 6.53 | 66 | 25 | 4 | 6 |
| CPP freeze/thaw 2x | 6.59 | 65 | 25 | 4 | 6 |

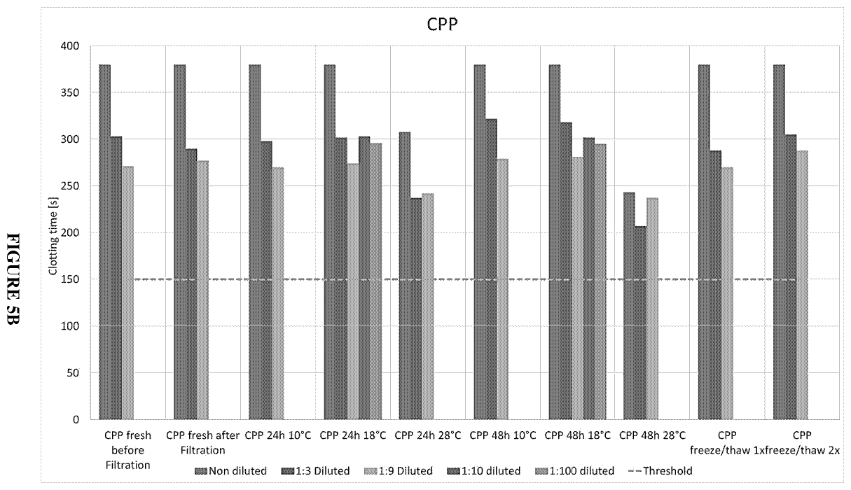

To determine clotting of proteins in plasma and CPP, pro-coagulant activity was determined using non-activated partial thromboplastin time (NaPTT) assay. The coagulation time was set at >150s in the NaPTT assay. Pro-coagulant activity was observed in plasma and CPP at 28° C. for 24 or 48 hours (FIG. 5). No pro-coagulant activity was observed in plasma and CPP filtered, freeze/thawed, or at a temperature of 10° C. or 18° C. for 24 or 48 hours.

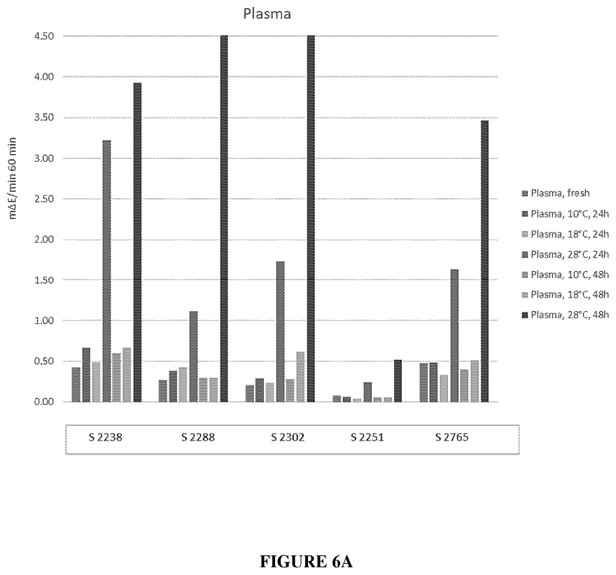

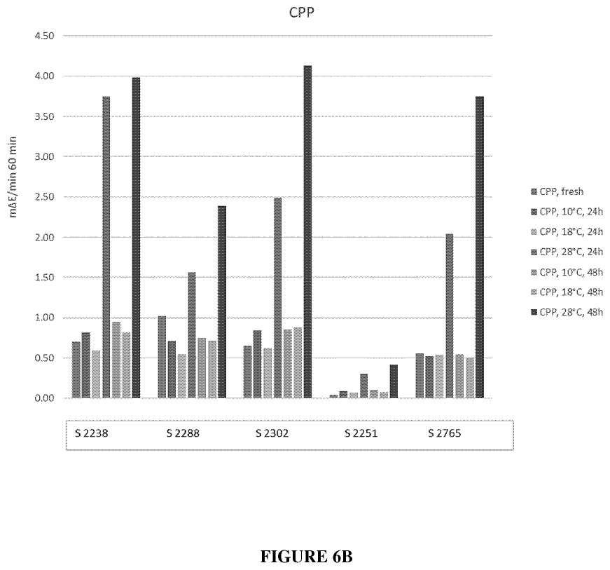

To determine proteolytic activity in plasma and CPP, the activity of thrombin, general serine proteases, kallikrein, plasmin and FXa was determined using chromogenic substrate assays (thrombin: S-2238; general serine proteases: S-2288; kallikrein: S-2302; plasmin: S-2251; and FXa: S-2765). Proteolytic activity was observed in plasma and CPP at 28° C. for 24 or 48 hours (FIG. 6). No proteolytic activity was observed in plasma and CPP filtered, freeze/thawed, or at a temperature of 10° C. or 18° C. for 24 or 48 hours.

Stability of the CRP and CPP was also assessed by determining changes in particle size within the sample as determined by microflow imaging (MFI) and dynamic light scattering (DLS) and the polydispersity index of the samples were also calculated. Tables 8 and 9 show the samples were broad polydispersed (>0.4). Tables 7 and 8 show that there was no difference in the polydispersity index of samples at 10° C. and 18° C. at 4 hr or 24 hr but a clear increase at 48 hr. The propensity index of plasma was not significantly different over temperatures and time suggesting that plasma samples are more stable over time and temperature compared to CPP.

TABLE 7
Particle analysis of CPP with and without filtration
and storage at set times and temperatures.

| | Time | Temperature | Polydispersity |
|---|---|---|---|
| Sample | (hr) | (° C.) | index |
| CPP before filtration | 0 | | 0.8022 |
| CPP after filtration | 0 | | 0.5168 |
| CPP | 4 | 10 | 0.506 |
| CPP | 4 | 18 | 0.5216 |
| CPP | 4 | 28 | 0.5673 |
| CPP | 24 | 10 | 0.5268 |
| CPP | 24 | 18 | 0.5391 |
| CPP | 24 | 28 | 0.7055 |
| CPP | 48 | 10 | 0.8131 |
| CPP | 48 | 18 | 0.8005 |
| CPP | 48 | 28 | 0.667 |

TABLE 7
Particle analysis of CRP with and without filtration
and storage at set times and temperatures.

| | Time | Temperature | Polydispersity |
|---|---|---|---|
| Sample | (hr) | (° C.) | index |
| Plasma before filtration | 0 | | 0.5271 |
| Plasma after filtration | 0 | | 0.5399 |
| Plasma | 4 | 10 | 0.4952 |
| Plasma | 4 | 18 | 0.5407 |
| Plasma | 4 | 28 | 0.4904 |
| Plasma | 24 | 10 | 0.5088 |
| Plasma | 24 | 18 | 0.548 |
| Plasma | 24 | 28 | 0.6498 |
| Plasma | 48 | 10 | 0.4935 |
| Plasma | 48 | 18 | 0.5778 |
| Plasma | 48 | 28 | 0.5153 |

The results show that particle formation occurs within the CRP and CPP with storage at higher temperatures and over longer periods of time. A suitable temperature to store the plasma or fractions thereof can be between 10° C. and 18° C. for up to 48 hrs.

### Example 7: Evaluation of Viral Inactivation Using n-Octyl-β-D-Glucopyranoside

N-Octyl-β-D-Glucopyranoside (OG) was evaluated to determine its virus reducing capacity in CRP prior to IgG purification. A thawed and homogenised CRP (50 mg/ml) and EMEM medium (control) was spiked with of Vesicular stomatitis virus (VSV) at 1:20 dilution. Aliquots of VSV spiked CRP and VSV spiked EMEM medium prior to OG addition were collected. OG was added to the VSV spiked CRP and VSV spiked EMEM medium such that the final OG concentration in the mixture was 30 mM, and mixed by pipetting for approximately 10 seconds. The mixture, along with the VSV spiked EDEM medium and VSV-contaminated plasma without OG were incubated at 5° C. Incubated samples were collected at 15, 30 and 60 minute time points. Samples were diluted 10-fold in medium to neutralise the activity of OG.

Aliquots of 100 μL of OG treated VSV spiked CRP, OG treated VSV spiked EDEM medium, VSV spiked CRP, VSV spiked EDEM medium and controls were titrated (ten-fold serial dilutions to 10⁻⁶ dilution) onto 150PL pre-cultured suspension of African Green Monkey Kidney Cells (Vero-PH) in standard 96-well microplates (Nunc, flat bottomed wells). Negative controls used were CRP and EDEM medium. Positive controls used were VSV stock used for spiking, and control virus stock with acceptable VSV titers achieved based on previous results from VSV virus stock characterization.

Plates were incubated at 37° C., 3-5% C02 and examined for virus specific cytopathogenic effect (CPE) using microscopy over 7 days. Cell cultures titrated with the negative controls were required to be free of CPE.

The infectivity titers were calculated according to the Spearman-Karber method and expressed as log 10 CCID50/mL (50% cell culture infectious dose per mL). If no infectious virus was detected by microtitration e.g. starting from a 1:10 dilution the virus tier is given as <1.5 log 10 CCID50/mL. To lower the detection limit, 1 mL of the 1:10 diluted post-treated Vero-PH cells was inoculated into 4 T25 flasks. When all 4 T25 cultures remain negative for infectious virus, the resulting infectivity titers is recorded as <0.5 log 10 CCID50/mL.

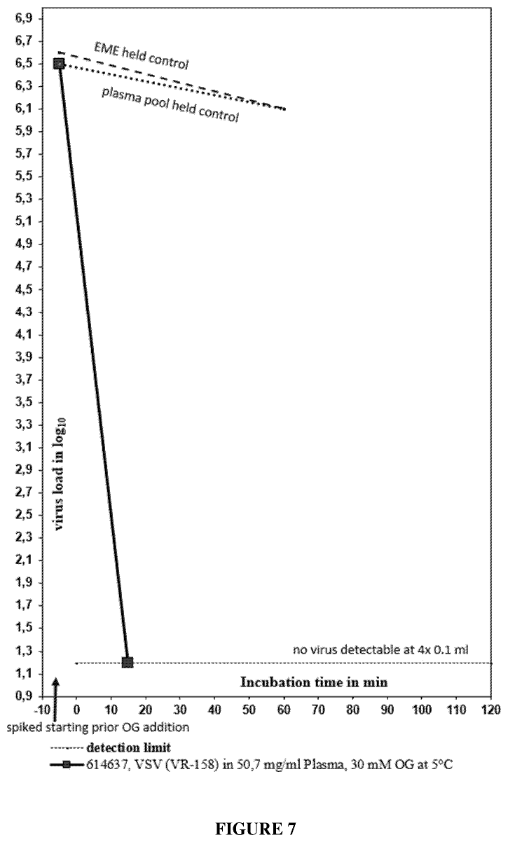

Robust reduction of VSV was observed where a final OG concentration of 30 mM was present in the OG treated VSV spiked CRP incubated at 5° C. The log 10 reduction factor (LRF) under these conditions (i.e. 30 mM final concentration of OG at 5° C.) was ≥5.3 (FIG. 7). LRF is the ratio of the virus load in the pre-treated starting material (e.g. CRP) and the virus load in the post-treated final material (e.g. OG treated CRP) and considers both sample volume and virus titres before and after treatment.

### Example 8: Optimization of Plasma Thawing

To evaluate the optimum temperature for plasma thawing, the temperature at which cryoprecipitates were observed was determined. Briefly, cryo-rich plasma (CRP) was gradually heated after thawing from 15° C. to 38° C. and at 20° C., 25° C., 28° C., 30° C., 32° C. and 37° C. a fraction of CRP was centrifuged and temperature dependent pellets weighed. At −30° C., only unspecific aggregates and no cryoprecipitate were present.

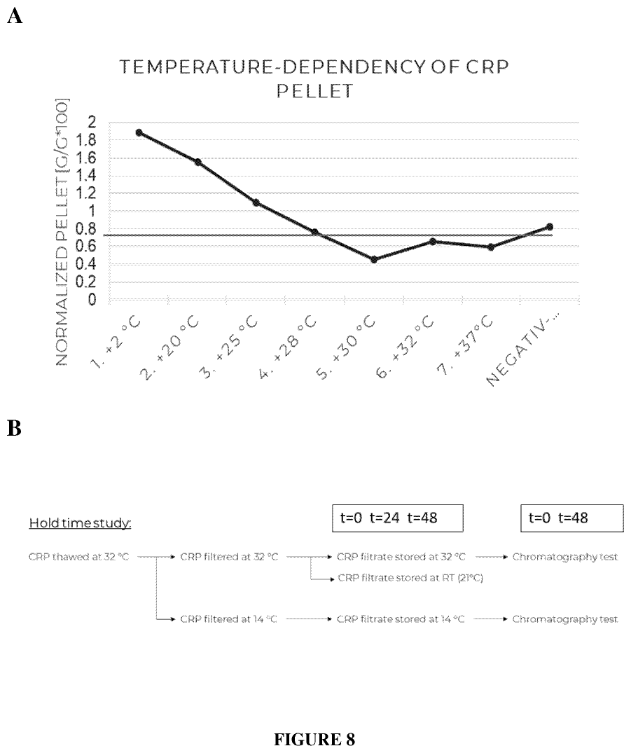

To determine the optimum temperature, the activity of von-Willebrand Factor (vWF), one of the main components of cryoprecipitate, was determined in each pellet. As shown in FIG. 8A and Table 8, thawing at 32° C. results in robust pellet formation and activities of vWF in the supernatant.

TABLE 8
vWF activity assay in CRP pellets at different temperatures

| Sample (temperature) | CBA:vWF (IU/mL) |
|---|---|
| vWF plasma | 0.080 |
| SHPL | 1.100 |

| | | |
|---|---|---|
| +2° | C. | <0.11 |
| +20° | C. | 1.098 |
| +20-+37° | C. | 1.070 |
| +25° | C. | 1.370 |
| +28° | C. | 1.455 |
| +30° | C. | 1.338 |
| +32° | C. | 1.218 |
| +37° | C. | 1.385 |

| | |
|---|---|
| +37-+60° C. | 0.290 |
| (negative ctrl) | |

To further confirm plasma thawing at 32° C., a hold time study was performed as set out in FIG. 8B and proteolytic activity determined using a NaPTT assay on the resulting eluate.

As shown in Table 9, no proteolytic activity was observed up to 48 hours when the eluate was filtered at 32° C.

TABLE 9
Proteolytic activity following CRP thawing at 32° C. versus 14° C.

| | NaPTT (Clotting time (seconds) | | | |
|---|---|---|---|---|
| | Non- | 1:3 | 1:10 | 1:100 |
| Sample | diluted | diluted | diluted | diluted |
| CRP at 0.5° C. | >380 | 288 | 282 | 286 |
| CRP thawed at 37° C., | >380 | 318 | 298 | 296 |
| before filtration | | | | |
| CRP thawed at 37° C., | >380 | 35 | 295 | 297 |
| after filtration | | | | |
| CRP thawed at 32° C., | >380 | 327 | 305 | 309 |
| before filtration | | | | |
| CRP 14° C. filtrate t = 0 | >380 | 309 | 301 | 317 |
| CRP 32° C. filtrate t = 0 | >380 | 353 | 330 | 331 |
| CRP 32° C. filtrate ->21° C. | >380 | 369 | 351 | 350 |
| hold t = 0 | | | | |
| CRP 14° C. filtrate t = 24 | >380 | 276 | 266 | 267 |
| CRP 32° C. filtrate t = 24 | >380 | 276 | 264 | 264 |
| CRP 32° C. filtrate ->21° C. | >380 | 270 | 261 | 272 |
| hold t = 24 | | | | |
| CRP 14° C. filtrate t = 48 | >380 | 338 | 292 | 285 |
| CRP 32° C. filtrate t = 48 | >380 | 255 | 264 | 296 |
| CRP 32° C. filtrate ->21° C. | >380 | 307 | 293 | 302 |
| hold t = 48 | | | | |

Samples were also analysed for levels of particulates using dynamic light scattering. No difference in polydispersity index (PDI) was observed between samples with a PDI<0.6 in all samples (data not shown).

Turbidity was also assessed over time and visible degradation for all temperatures (i.e., 14° C., 21° C. and 32° C.) after 48 hours was observed. At 24 hours no visible degradation was observed at 14° C., compared with most at 32° C. For plasma held at 32° C. a distinct cycle per cycle pressure increase during chromatography was also observed at 48 hours, compared to 14° C. (data not shown).

Despite visible degradation, no impact on IgG content was observed. In particular, there was no reduction in IgG content over time when samples were thawed and stored at higher temperatures.

### Example 9: Optimization of Continuous Affinity Chromatography Method

### Removal of Strip Phase

During the SMB process, an increase in back pressure of single columns was observed due to denaturation of accumulated proteins on the column by the harsh conditions (i.e., pH2.5) during the strip phase. Accordingly, to investigate whether removal of the strip phase reduced the back pressure increase, the SMB process was run without the strip phase.

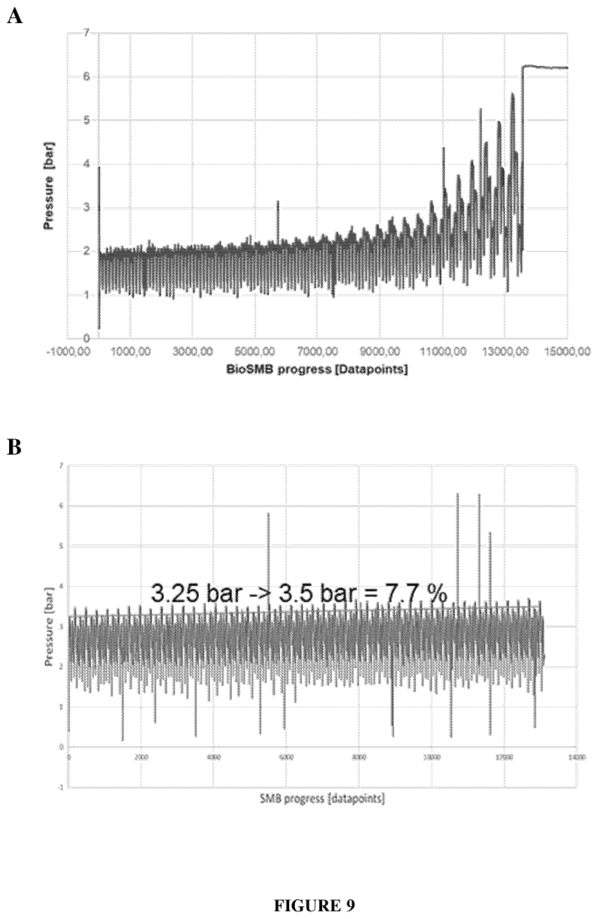

As shown in FIG. 9, removal of the strip phase resulted in a reduction in the back pressure increase and stabilization of the pressure during the SMB process.

### Increasing Conductivity of Wash Phase

The conductivity of the wash phase was screened to investigate whether the protease activity in the eluate could be reduced.

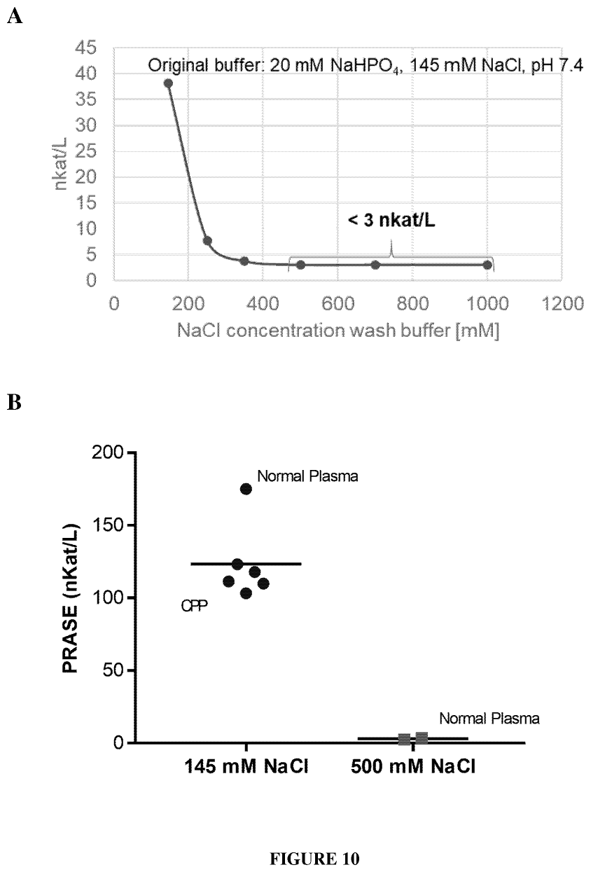

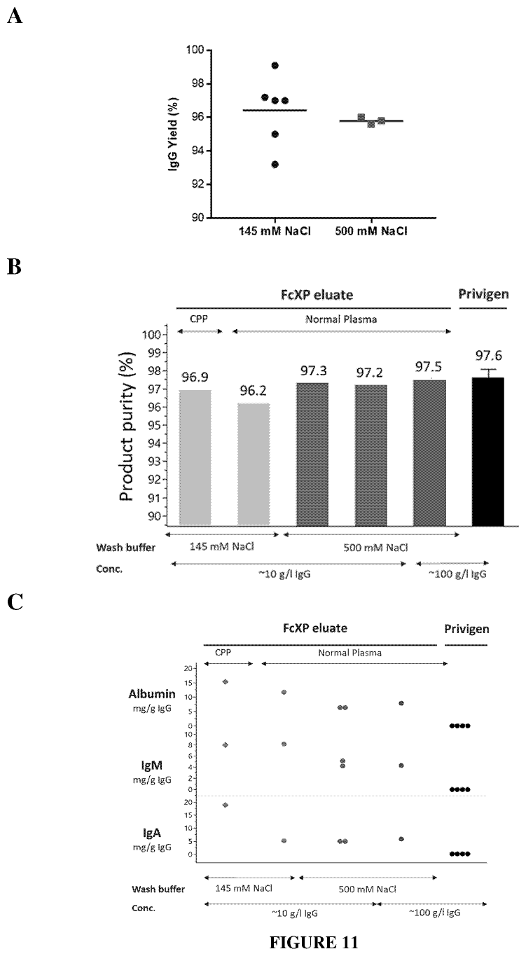

As shown in FIG. 10, increasing the concentration of the sodium chloride in the wash buffer from 145 mM to 500 mM reduced the protease activity in the eluate below the limit of detection. Increasing the conductivity of the wash buffer increased the elution of additional proteins (e.g., components of the complement system) however no effect on IgG yields and/or overall product purity was observed with the higher conductivity wash buffer (FIG. 11). Purity levels were comparable to commercially available Privigen by Labchip assay and nephelometry (FIGS. 11B and 11C).

### Example 10: Anion Exchange Polishing Step

The use of the POROS™-HQ 50 anion exchange resin as a polishing step was evaluated. The resin was operated in flow through mode.

Initially, the resin was screened using MES and phosphate buffers for impact on impurity depletion.

FcXP eluate was subjected to UF/DF in 10 mM MES or phosphate buffer pH 6.0, 0 mM NaCl prior to loading on the anion exchange chromatography column. Flow through and post-wash were collected and analysed separately.

As shown in Table 10, MES buffer and phosphate buffer resulted in acceptable IgG yield, and acceptable impurity depletion.

Phosphate buffer at pH 6.0 (low conductivity) yielded the best results in terms of development targets of impurity depletion.

TABLE 10
Buffer screening

| | | | | | | | | | Depletion Factor (with PW) | | |
|---|---|---|---|---|---|---|---|---|---|---|---|
| | pH at end of | | | IgG Yield % (Neph) | | Depletion factor (excluding PW) | | | IgA | IgM | Albumin |
| | equilibration | | NaCl | Without | With | IgA | IgM | Albumin | (Target | (Target | (Target |
| Buffer | before loading | pH | (mM) | postwash | postwash | (Neph) | (Neph) | (Neph) | 108) | 1153) | 541) |
| MES | 5.2 | 6.0 | 20 | 86.9 | 93.4 | 273 | 805 | 117 | 228 | 305 | 90 |
| MES | 6.2 | 6.0 | 50 | 87.8 | 95.2 | 66 | >1498 | 39 | 53 | >1076 | 32 |
| MES | 6.7 | 6.6 | 25 | 82.1 | 88.5 | 360 | 518 | 321 | 252 | 225 | 200 |
| MES | 6.7 | 6.6 | 50 | 83.8 | 90.1 | 84 | >1726 | 66 | 66 | >1208 | 52 |
| Phosphate | 3 | 6.0 | 0 | 78.0 | | 147 | >1476 | 88 | | | |
| Phosphate | 4.6 | 6.0 | 50 | 84.1 | | 40 | 1120 | 2 | | | |
| Phosphate | 6.56 | 6.6 | 50 | 75.5 | | 77 | 498 | 2 | | | |

### Example 11: Anion Exchange Polishing Step Equilibration and Load Buffer Optimization Study and Design of Experiments (DoE) Study

### Buffer Evaluation Study

### Buffers and Conditions Assessed:

| Ionic state | | |
|---|---|---|
| of buffer | | |
| counter ion | Buffers | Conditions |
| Anionic | Sodium citrate | pH 6.0, 0 and 50 mM NaCl |
| | | pH 6.6, 0 and 50 mM NaCl |
| Anionic | MES | pH 6.0, 0 mM NaCl |
| | | pH 6.6, 0 mM NaCl |
| Anionic | Sodium dihydrogen phosphate | pH 6.6, 0 mM NaCl |
| Cationic | Bis-Tris | pH 6.0, 0 and 50 mM NaCl |
| | | pH 6.6, 0 and 50 mM NaCl |
| Cationic | L-Histidine | pH 6.0, 0 and 50 mM NaCl |
| | | pH 6.6, 0 and 50 mM NaCl |

Samples were loaded according to Example 10 above. Briefly, FcXP eluate was rebuffered and loaded onto Poros™ HQ5. Flow though and post-wash were collected and analysed separately.

Citrate buffer depleted IgA well in a broad range of both NaCl and pH. Acceptable IgA depletion was achieved in phosphate at low salt.

Phosphate buffer depleted IgM well in a broad range of both NaCl and pH.

Bis-Tris and histidine depleted albumin well in a broad range of both NaCl and pH. Good albumin depletion in phosphate at low salt.

### Design of Experiments Phosphate Buffer

The following conditions were assessed:

| | Phase | |
|---|---|---|
| | length | |
| Phase | (CV) | Composition |
| Equilibration | 15 | 10 mM NaH₂PO₄ + x mM NaCl, pH y |
| Load | | 5-15 g/L IgG, varied loading |
| Postwash | 3 | 10 mM NaH₂PO₄ + x mM NaCl, pH y |
| Regen. - high salt | 5 | 1M NaCl, 10 mM NaH₂PO₄, pH 6.2 |
| Regen - NaOH | 5 | 0.5M NaOH |
| Regen. - Hac | 5 | 1M HAc |
| Regen. - NaCl, pH 6.2 | 5 | 1M NaCl, 100 mM NaH₂PO₄, pH 6.2 |

x: 0-40 mM
y: pH 5.8-6.6

Preliminary recommendations for equilibration and loading include 0 mM NaCl and pH 6.2, with load material in the pilot scale run having a purity of ˜93.5% and an impurity concentration of 40.4-71.6 mg/g IgG. In the BioSMB run, the load material had a purity of ˜97%, an impurity/IgG concentration of 12.0-20.4 mg/g IgG and concentration of 5-15 g/L IgG for best impurity depletion. The load of the resin was 25-42 mg impurities per mL resin.

Preliminary recommendations for post-wash conditions include 0 mM NaCl and pH 6.0.

### Example 12: Anion Exchange Polishing Step

The MES and phosphate buffers were further screened for impact on impurity depletion in the POROS™ HQ 50 anion exchange polishing step. The process was performed as described in Example 10 above using MES buffer pH 6.0 with 20 mM or 50 mM NaCl, MES buffer pH 6.6 with 25 mM or 50 mM NaCl, and phosphate buffer pH 6.2 with 0 mM NaCl.

MES buffer and phosphate buffer resulted in acceptable IgG yield, and acceptable impurity depletion in the flow through (without post wash).

| | | | IgM | IgA | Albumin |
|---|---|---|---|---|---|
| Buffer | pH | NaCl | (μg/g IgG) | (mg/g IgG) | (mg/g IgG) |
| MES | 6.0 | 20 mM | 5.18 | 0.05 | 0.41 |
| | | 50 mM | ≤2.73 | 0.22 | 1.26 |
| | 6.6 | 25 mM | 7.80 | 0.04 | 0.15 |
| | | 50 mM | ≤2.32 | 0.17 | 0.74 |
| Phosphate | 6.2 | 0 mM | ≤3.07 | 0.07 | 0.43 |

### Example 13: Scale-Up of SMB FcXP Chromatography Step

A 4-column set up in SMB mode with 1 cm inner diameter columns was performed without a resin strip phase and alternate wash phase using the following conditions:

| | | Phase | Flow | Residence |
|---|---|---|---|---|
| | | length | rate | time |
| Phase | Input | (CV) | (cm/h) | (mins) |
| Product | Feed/Load | 4 | 642 | 0.56 |
| loading | (clarified | | | |
| | plasma pool) | | | |
| Wash | Buffer 1 | 2.75 | 856 | 0.42 |
| Pre elution | Buffer 2 | 0.6 | 856 | 0.42 |
| Elution | Buffer 2 | 2.2 | 1286 | 0.28 |
| Post elution | Buffer 1 | 0.7 | 1286 | 0.28 |
| Equilibration | Buffer 1 | 3 | 1286 | 0.28 |

Buffer 1: 20 mM NaH₂PO₄, 500 mM NaCl, pH 7.4;
Buffer 2: 20 mM acetic acid, pH 4.0

After each batch, all columns were regenerated using 20% EtOH and 20 mM NaOH and used for processing the next batch.

The IgG yield in FcXP eluate was 98.3-99.1%, with total IgG recovery in all fractions 99.4-100.3%. Purity of the FcXP eluate was also assessed by Labchip, with product purity being between 96.4-96.9%.

The purification process resulted in 9.55-10.4 g/L IgG.

Impurities in the FcXP eluate was determined including IgM (47.85-51.90 mg/L; 4.8-5.3 mg/gm IgG), IgA (68.8-76.55 mg/L; 7-8 mg/gm IgG) and albumin (55.3-78.05 mg/L; 5.8-7.6 mg/gm IgG).

### Example 14: Scaled-Up Process Runs Using Cryo-Rich Plasma

Pooled cryo-rich plasma from 30 donors was thawed and clarified by 1.2 μm and 0.45 μm+0.2 μm filters in series before being used for downstream processing.

Thawed and filtered plasma was purified and processed according to the following process:
1. FcXP SMB chromatography
2. Concentration and buffer exchange via UF/DF
3. pH shift and filtration
4. Anion exchange chromatography (using POROS™-HQ50)
5. Isoagglutinin affinity chromatography
6. Virus inactivation
7. UF/DF
8. Formulation to 100 g/L IgG

Three separate runs were performed with IgG recovery from steps 1-6 being 83-96% and total process recovery between 81-94%.

The purification process resulted in IgG subclass distribution in the final formulated bulk product of 67-69% IgG1, 24-27% IgG2, 3-4% IgG3 and 2-3% IgG4.

Purity of the product following isoagglutinin chromatography was also assessed by Labchip, with product purity being between 97-98.5%.

The POROS 50HQ flow through and post wash was assessed for impurities including IgM (<1.86-<2.55 μg/gm IgG), IgA (0.109-0.111 mg/gm IgG) and albumin (0.59-0.74 mg/gm IgG). Yield was also determined as 93-97%.

The formulated bulk product was also assessed for impurities including IgM (<0.17 mg/L; <1.74-<1.86 μg/gm IgG), IgA (8.46-8.76 mg/L; 0.089-0.093 mg/gm IgG) and albumin (62.75-67.60 mg/L; 0.64-0.74 mg/gm IgG). FcXP ligand was <10 ppm (below the limit of detection).

Isoagglutinin depletion was also assessed after FcXP SMB chromatography (Iso-A titer 1:16; Iso-B titer 1:32), after POROS HQ50 chromatography (Iso-A titer 1:4; Iso-B titer 1:8), and after isoagglutinin chromatography (Iso-A titer 1:0; Iso-B titer 1:0).

Sequences

SEQ ID NO: 1 is an amino acid sequence of VHH
fragment
QVQLQESGGGLVQAGDSLRISCRASGLTVNDLYMGWFRQAPGKEREFV
GRVTPGDNTDYTYYVDSVKGRFTISRDSAKNTVYLQMNSLKLEDTAVY
LCAGRRFGSSEWDYWGQGTQVTVSS
SEQ ID NO: 2 is an amino acid sequence of CDR1
of the VHH fragment
GLTVNDLYMG
SEQ ID NO: 3 is an amino acid sequence of CDR2
of the VHH fragment
RVTPGDNTDYTYYVDSVK
SEQ ID NO: 4 is an amino acid sequence of the
VHH fragment
RRFGSSEWDY

1. A method of purifying immunoglobulin G (IgG) from plasma or a fraction thereof using continuous affinity chromatography, the method comprising binding the IgG to an affinity chromatography resin comprising a ligand capable of specifically binding to a CH3 domain of human IgG and collecting the IgG.

2. A method of producing an immunoglobulin G (IgG) enriched preparation from plasma or a fraction thereof using continuous affinity chromatography, the method comprising binding the IgG to an affinity chromatography resin comprising a ligand capable of specifically binding to a CH3 domain of human IgG and collecting the IgG.

3. The method of claim 1, wherein the ligand comprises a camelid-derived single domain [VHH] antibody fragment.

4. The method of claim 1, wherein the resin comprises a matrix selected from the group consisting of a cross-linked poly(styrene-divinylbenzene) matrix and an agarose-based matrix.

5. (canceled)

6. The method of claim 1, wherein the method further comprises washing the resin with a wash buffer having a pH of between 5 and 10 and a dissociation constant (pKa) between 6.8 and 8.5 at 25° C.

7. The method of am claim 1, wherein the method further comprises eluting the bound IgG from the resin with an elution buffer having a pH of between 3 and 5.

8. The method of claim 1, wherein the plasma or fraction thereof contacts the resin for between 0.1 and 5 minutes.

9. The method of claim 1, wherein the plasma fraction is selected from a group consisting of cryo-rich plasma, cryo-poor plasma, Supernatant I (SN I), Cohn Fraction II (Fr II), Cohn Fraction II+III (Fr II+III), Cohn Fraction I+II+III (FrI+II+III), Kistler/Nitschmann Precipitate A (KN A), Kistler/Nitschmann Precipitate B (KN B), Kistler/Nitschmann Precipitate of Supernatant B (KN B+1), and combinations thereof.

10. The method of claim 1, wherein the plasma or fraction thereof is thawed at a temperature of at least 32° C.

11. The method of claim 1, wherein the plasma or fraction thereof is at a temperature in the range of from 2° C. to 28° C. before the continuous affinity chromatography.

12.-13. (canceled)

14. The method of claim 1, wherein the wash buffer comprises a buffering agent selected from a group consisting of sodium dihydrogen phosphate, imidazole, Tris, glycylglycine, 3-morpholinopropane-1-sulfonic acid (MOPS), piperazine-N,N′-bis(2-ethanesulfonic acid) (PIPES), 2-[(2-Hydroxy-1,1-bis(hydroxymethyl)ethyl)amino]ethanesulfonic acid (TES), bis[(2-hydroxyethyl)amino]acetic acid (Bicine), 4-(2-hydroxyethyl)-1-piperazineethanesulfonic acid (HEPES), sulfurous acid, 4-(2-Hydroxyethyl)-1-piperazinepropanesulfonic acid (EPPS), N-(Hydroxyethyl)piperazine-N′-2-hydroxypropanesulfonic acid (HEPPSO), 4-(N-Morpholino)butanesulfonic acid (MOBS), Piperazine-N,N′-bis(2-hydroxypropanesulfonic acid) (POPSO), N-[Tris(hydroxymethyl)methyl]-3-amino-2-hydroxypropanesulfonic acid (TAPSO), Tricine, triethanolamine (TEA) and combinations thereof.

15. (canceled)

16. The method of claim 1, wherein the wash buffer further comprises sodium chloride and/or a divalent salt at a concentration of up to 1000 mM.

17. (canceled)

18. The method of claim 1, wherein the elution buffer is or comprises a phosphate buffer and/or an acetate buffer at a pH of between 3 and 5.

19. (canceled)

20. The method of claim 1, wherein the method further comprises equilibrating the resin with an equilibration buffer having a pH between 7 and 8.

21.-22. (canceled)

23. The method claim 1, wherein the method further comprises equilibrating the resin after stripping the resin with an equilibration buffer having a pH between 7 and 8.

24. The method of any claim 1, wherein the continuous affinity chromatography is selected from the group consisting of simulated moving bed (SMB) chromatography, periodic counter-current chromatography (PCC), continuous counter-current tangential chromatography (CCTC), and continuous counter-current spiral chromatography (CCSC).

25. The method of claim 1, wherein the resin is in the form of a slurry or the resin is packed into one or more column(s), wherein each column comprises one or more zones.

26. The method of claim 1, wherein the resin is in the form of a slurry or the resin is packed into one or more column(s), wherein each column comprises one or more zones chosen from an equilibration zone, a binding zone, a wash zone, an elution zone or a combination thereof.

27. The method of claim 1, wherein the resin is packed into one or more column(s) and wherein the columns are fluidly connected and separated by fluid conduits comprising inlet and outlet valves.

28. The method of claim 1, wherein the resin is packed into one or more column(s), and wherein the resin is packed into a series of three columns, wherein each column is a separate zone.

29. (canceled)

30. The method of claim 25, wherein at least one column is loaded with IgG at a concentration above the dynamic binding capacity (DBC) of the resin.

31.-33. (canceled)

34. The method of claim 1, wherein the resin has a total bed height of at between 2 cm and 30 cm.

35. The method of claim 1, wherein the method further comprises regenerating the resin.

36. The method of claim 1, wherein the method further comprises sanitizing the resin.

37. The method of claim 1, wherein the method further comprises one or more steps selected from a group consisting of ethanol precipitation, octanoic acid fractionation, ion exchange chromatography, viral inactivation, viral filtration and ultrafiltration/diafiltration.

38. The method of claim 1, wherein the method further comprises an ion exchange chromatography step, wherein the ion exchange chromatography step comprises anion exchange chromatography step using a strong anion exchange resin operated in flow through mode.

39.-43. (canceled)

44. The method of claim 1, wherein at least 75% of the IgG is recovered from the plasma or fraction thereof.

45. The method of claim 1, wherein the eluted IgG has a purity of at least 95%.

46. (canceled)

47. A method for purifying immunoglobulin G (IgG) from plasma or a fraction thereof using simulated moving bed (SMB) chromatography, the method comprising:
a) equilibrating an affinity chromatography resin comprising a cross-linked poly(styrene-divinylbenzene) matrix and a ligand capable of specifically binding to a CH3 domain of human IgG with a 20 mM phosphate equilibration buffer having a pH of between 7 and 8;
b) binding the IgG from the plasma or fraction thereof to the resin;
c) washing the resin with a 20 mM phosphate wash buffer having a pH of between 7 and 8; and
d) eluting the bound IgG with a 20 mM acetate or phosphate elution buffer having a pH of between 3 and 5;
wherein steps a) to d) may be repeated on the affinity chromatography resin and wherein, the affinity chromatography resin is packed into a series of two or more fluidly-connected columns separated by fluid conduits comprising inlet and outlet valves, and optionally wherein the method does not comprise stripping the resin.

48. A pharmaceutical composition comprising IgG purified or produced by the method of claim 1.

49.-50. (canceled)

51. The pharmaceutical composition of claim 48, wherein the pharmaceutical composition comprises a purity of at least 98% immunoglobulin G (IgG).

52.-56. (canceled)

57. The pharmaceutical composition of claim 48, wherein the pharmaceutical composition comprises an IgA content of ≤0.05 mg/mL.

58. The pharmaceutical composition of claim 48, wherein the pharmaceutical composition comprises a Prekallikrein activator (PKA) level of ≤35 IU/mL.

59.-60. (canceled)

61. A method of treating, preventing and/or delaying progression of a condition in a subject, the method comprising administering the pharmaceutical composition of claim 48 to the subject.

62. The method of claim 61, wherein the condition is selected from a group consisting of primary immunodeficiency disease (PI), chronic inflammatory demyelinating polyneuropathy (CIDP), and chronic immune thrombocytopenic purpura (ITP).


SEQUENCE LISTING (4 sequences)

SEQ ID NO: 1 - protein, 121 aa, Camelus sp.
QVQLQESGGGLVQAGDSLRISCRASGLTVNDLYMGWFRQAPGKEREFVGRVTPGDNTDYTYYVDSVKGRFTISRDSAKNTVYLQMNSLKLEDTAVYLCAGRRFGSSEWDYWGQGTQVTVSS

SEQ ID NO: 2 - protein, 10 aa, Camelus sp.
GLTVNDLYMG

SEQ ID NO: 3 - protein, 18 aa, Camelus sp.
RVTPGDNTDYTYYVDSVK

SEQ ID NO: 4 - protein, 10 aa, Camelus sp.
RRFGSSEWDY